(19) United States
(12) Patent Application Publication
(10) Pub. No.: US 2013/0109019 A1
(43) Pub. Date: May 2, 2013

(54) HAPTEN CONJUGATES FOR TARGET DETECTION

(71) Applicants: Adrian E. Murillo, Tucson, AZ (US); Jerome W. Kosmeder, Tucson, AZ (US); Eric May, Chandler, AZ (US); William Day, Tucson, AZ (US); Mark Lefever, Oro Valley, AZ (US); Anne M. Pedata, Tucson, AZ (US); Christopher Bieniarz, Tucson, AZ (US); Phillip Miller, Tucson, AZ (US)

(72) Inventors: Adrian E. Murillo, Tucson, AZ (US); Jerome W. Kosmeder, Tucson, AZ (US); Eric May, Chandler, AZ (US); William Day, Tucson, AZ (US); Mark Lefever, Oro Valley, AZ (US); Anne M. Pedata, Tucson, AZ (US); Christopher Bieniarz, Tucson, AZ (US); Phillip Miller, Tucson, AZ (US)

(21) Appl. No.: 13/805,978

(22) PCT Filed: Jul. 1, 2011
(86) PCT No.: PCT/US11/42849

§ 371 Date: Dec. 20, 2012

Related U.S. Application Data
(60) Provisional application No. 61/398,946, filed on Jul. 2, 2010.
(60) Provisional application No. 61/464,216, filed on Feb. 28, 2011.

(51) Int. Cl. C07D 277/54; C07C 237/20; C07D 271/12; C07D 233/90; C07D 243/00; C07D 241/52

(52) U.S. Cl. CPC C07D 277/54; C07D 243/00; C07D 241/52; C07D 271/12; C07D 233/90; C07C 237/20
USPC 435/6.11; 540/517; 544/355; 548/126; 548/185; 548/327.5; 564/155; 435/28; 435/7.1; 435/7.9

(57) ABSTRACT

Embodiments of hapten conjugates including a hapten, an optional linker, and a peroxidase-activatable aryl moiety are disclosed. In some embodiments, the peroxidase-activatable aryl moiety is tyramine or a tyramine derivative. Embodiments of methods for making and using the hapten conjugates also are disclosed. In particular embodiments, the hapten conjugates are used in a signal amplification assay. In certain embodiments, the hapten is an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, or 7-diethylamino-3-carboxycoumarin. The hapten is coupled to the peroxidase-activatable aryl moiety directly or indirectly via a linker. In certain embodiments, the hapten conjugates are used in multiplexed assays.

### CROSS REFERENCE TO RELATED APPLICATIONS

This application claims priority to U.S. Provisional Application No. 61/398,946 filed on Jul. 2, 2010, and U.S. Provisional Application No. 61/464,216 filed on Feb. 28, 2011, which are incorporated herein in their entirety.

### FIELD

This disclosure concerns haptens and hapten conjugates that can be utilized in various combinations for the simultaneous identification and/or visualization of a target in a sample.

### BACKGROUND

Immunohistochemistry, or IHC, refers to the process of localizing antigens, such as a protein, in cells of a tissue sample and using the antigens to promote specific binding of antibodies to the particular antigens. This detection technique has the advantage of being able to show exactly where a given protein is located within the tissue sample. It is also an effective way to examine the tissues themselves.

The use of small molecules such as haptens, to detect tissue antigens and nucleic acids has become a prominent method in IHC. Haptens, in combination with anti-hapten antibodies are useful for detecting particular molecular targets. For example, specific binding moieties such as primary antibodies and nucleic acid probes can be labeled with one or more hapten molecules, and once these specific binding moieties are bound to their molecular targets they can be detected using an anti-hapten antibody conjugate that includes an enzyme as part of a chromogenic based detection system or a detectable label such as a fluorescent label. Binding of the detectable anti-hapten antibody conjugate to a sample indicates the presence of the target in a sample.

Digoxigenin, present exclusively in Digitalis plants as a secondary metabolite, is an example of a hapten that has been utilized in a variety of molecular assays. U.S. Pat. No. 4,469,797 discloses using immunoassays to determine digoxin concentrations in blood samples based upon the specific binding of anti-digoxin antibodies to the drug in the test sample. U.S. Pat. No. 5,198,537 describes a number of additional digoxigenin derivatives that have been used in immunological tests, such as immunoassays.

For in situ assays such as immunohistochemical (IHC) assays and in situ hybridization (ISH) assays of tissue and cytological samples, especially multiplexed assays of such samples, it is highly desirable to identify and develop methods which provide desirable results without background interference. One such method involves the use of Tyramide Signal Amplification (TSA), which is based on the patented catalyzed reporter deposition (CARD). U.S. Pat. No. 6,593,100 discloses enhancing the catalysis of an enzyme in a CARD or tyramide signal amplification (TSA) method by reacting a labeled phenol conjugate with an enzyme, wherein the reaction is carried out in the presence of an enhancing reagent.

While methods have been employed to increase the signals obtained from assays using haptens, the results from these methods indicate that signal amplification is impaired by corresponding background signal amplification. A need exists for signal amplification that can produce optimal results without a corresponding increase in background signals.

### SUMMARY

Embodiments of hapten conjugates are disclosed. In some embodiments, the conjugates include a hapten, an optional linker, and a peroxidase-activatable aryl moiety. In certain embodiments, the peroxidase-activatable aryl moiety is tyramine or a tyramine derivative. Also disclosed are embodiments of methods for making and using the hapten conjugates.

In some embodiments, the hapten is selected from an azole (e.g., an oxazole, a pyrazole, a thiazole), a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, or a coumarin (e.g., 2,3,6,7-tetrahydro-11-oxo-1H,5H,11H-[1]benzopyrano[6,7,8-ij]quinolizine-10-carboxylic acid or 7-diethylamino-3-carboxycoumarin). The hapten may be coupled directly to a peroxidase-activatable aryl moiety, e.g., a tyramine or tyramine derivative. Alternatively, the hapten may be coupled via a linker to a tyramine or tyramine derivative. Thus, in certain embodiments, the conjugate has a general formula as shown below.

hapten-optional linker-tyramine/tyramine derivative

Embodiments of the disclosed hapten conjugates include a peroxidase-activatable aryl moiety capable of forming a free radical when combined with a peroxidase enzyme and peroxide and subsequently forming a dimer with a phenol-containing compound, e.g., tyrosine. The peroxidase-activatable moiety has a general formula

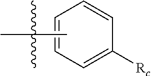

where R_{C} is a functional group capable of forming a free radical when combined with a peroxidase enzyme and peroxide. Suitable functional groups include hydroxyl, ether, amine, and substituted amine groups. In some embodiments, the peroxidase-activatable aryl moiety is tyramine

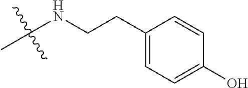 [SMILES: *NCCc1ccc(O)cc1]

or a tyramine derivative having the following general formula

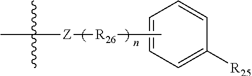

where R₂₅ is selected from hydroxyl, ether, amine, and substituted amine; R₂₆ is selected from alkyl, alkenyl, alkynyl, aryl, heteroaryl, —ORₘ, —NRₘ, and —SRₘ, where m is 1-20; n is 1-20; Z is selected from oxygen, sulfur, or NRₐ where Rₐ is selected from hydrogen, aliphatic, aryl, or alkyl aryl.

Certain embodiments of the disclosed hapten conjugates include a linker having the general formula

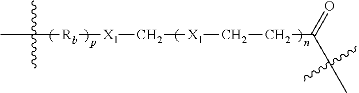

where each X₁ independently is selected from —CH₂, oxygen, sulfur, and —NR₃ where R₃ is selected from hydrogen, aliphatic, aryl, and aryl alkyl; R_{b} is selected from carbonyl and sulfoxyl; n is 1-20; and p is 0 or 1. In certain embodiments, the linker is a polyethylene glycol having a formula PEGₙ where n is 1-50, such as 4 or 8. In a particular embodiment, the linker has the following chemical structure.

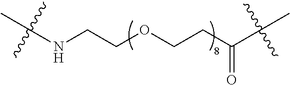

In some embodiments, the hapten conjugate is a hapten-tyramide conjugate having a formula selected from:

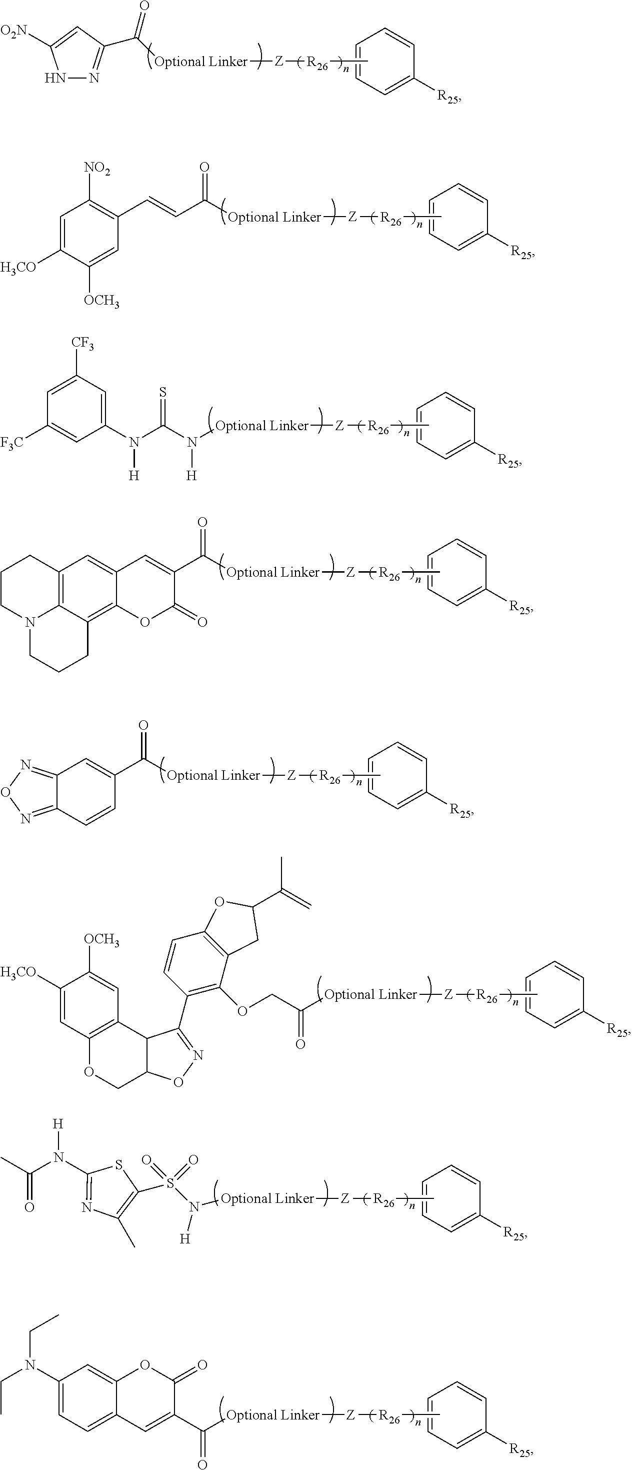

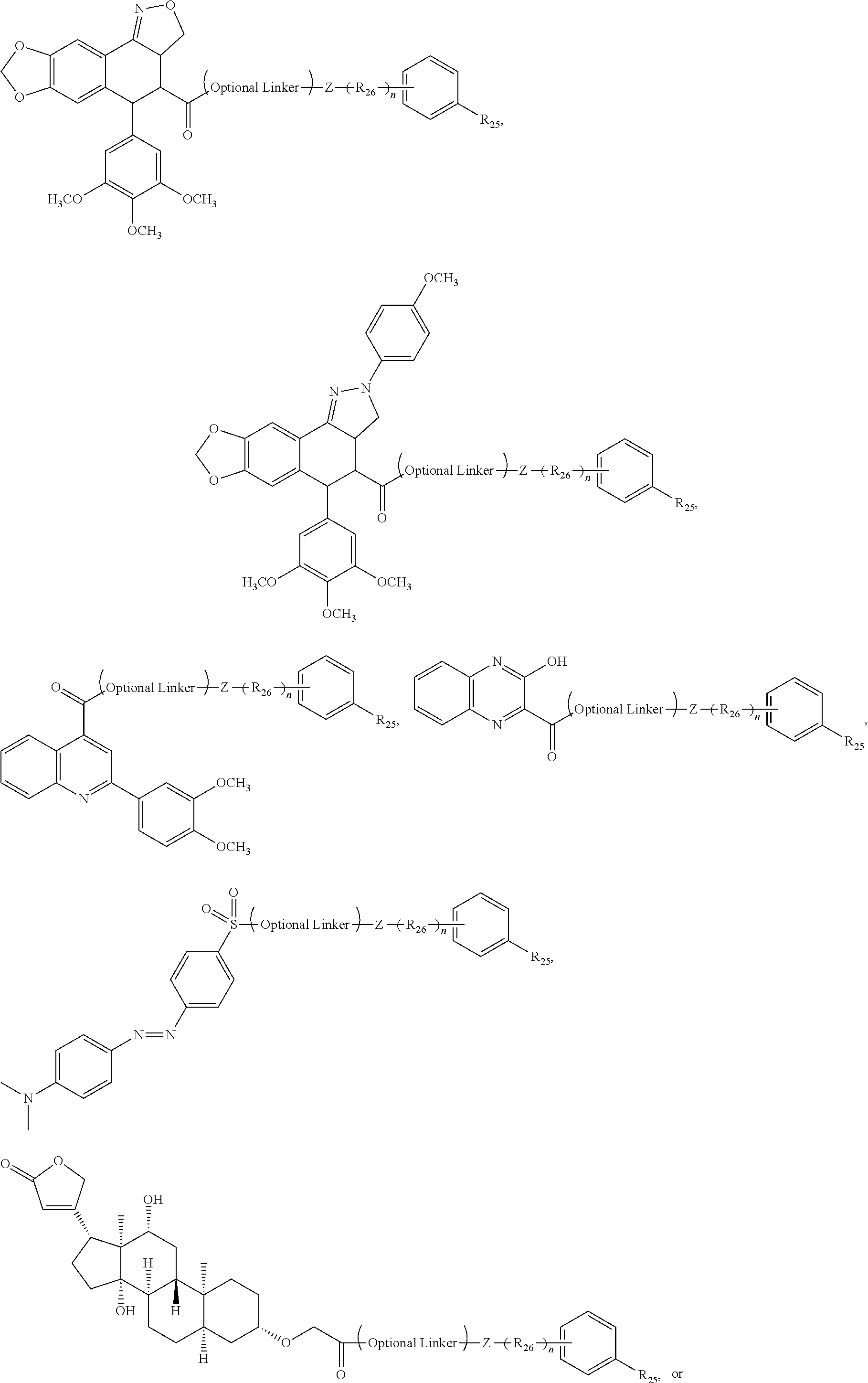

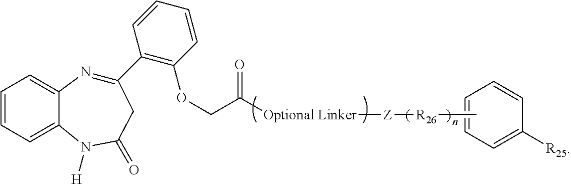

Exemplary hapten-tyramide conjugates include:

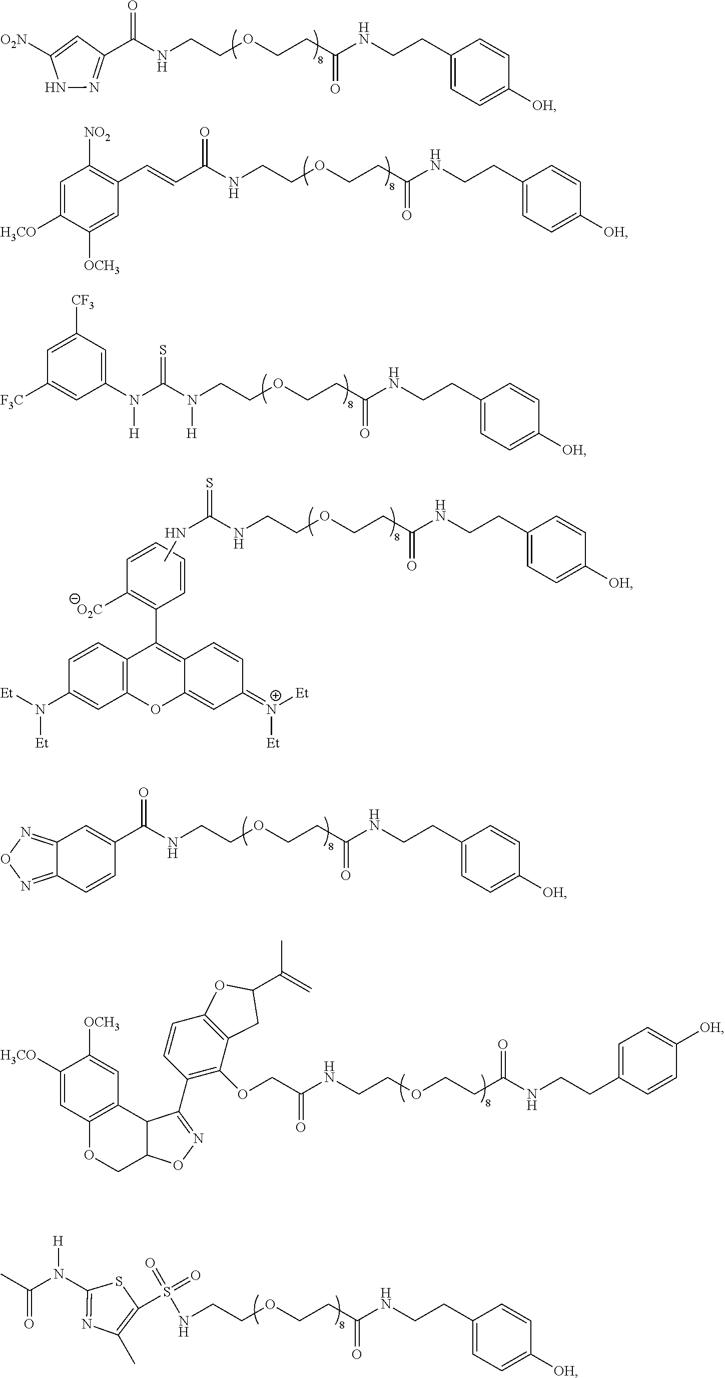

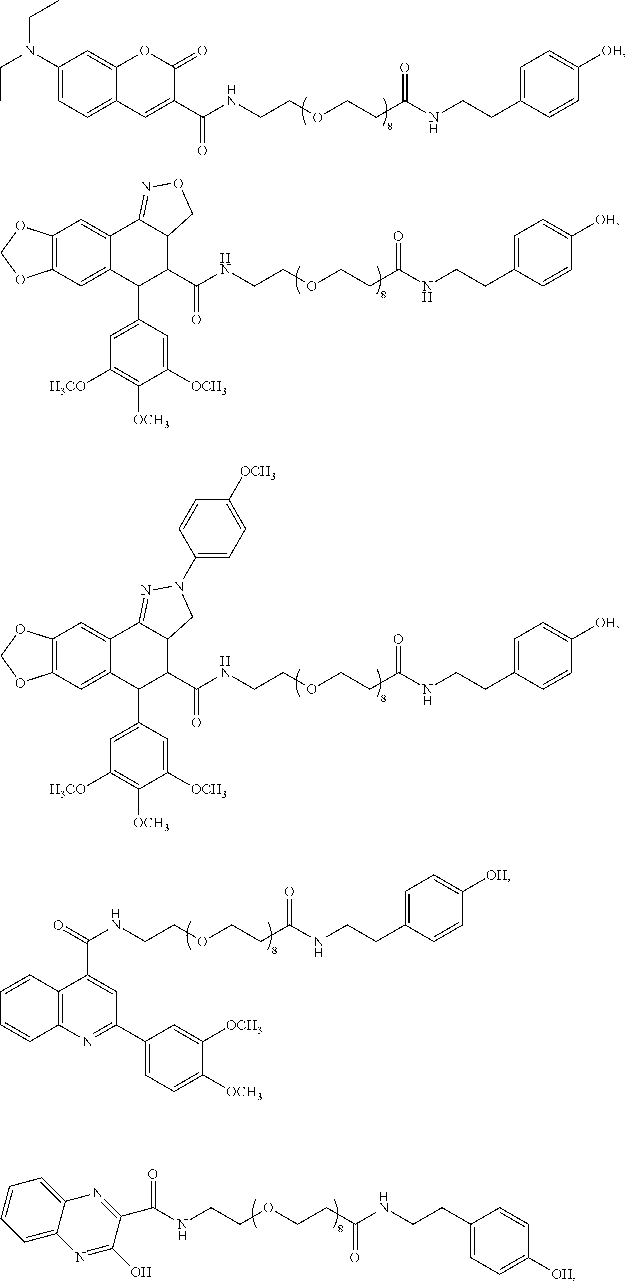

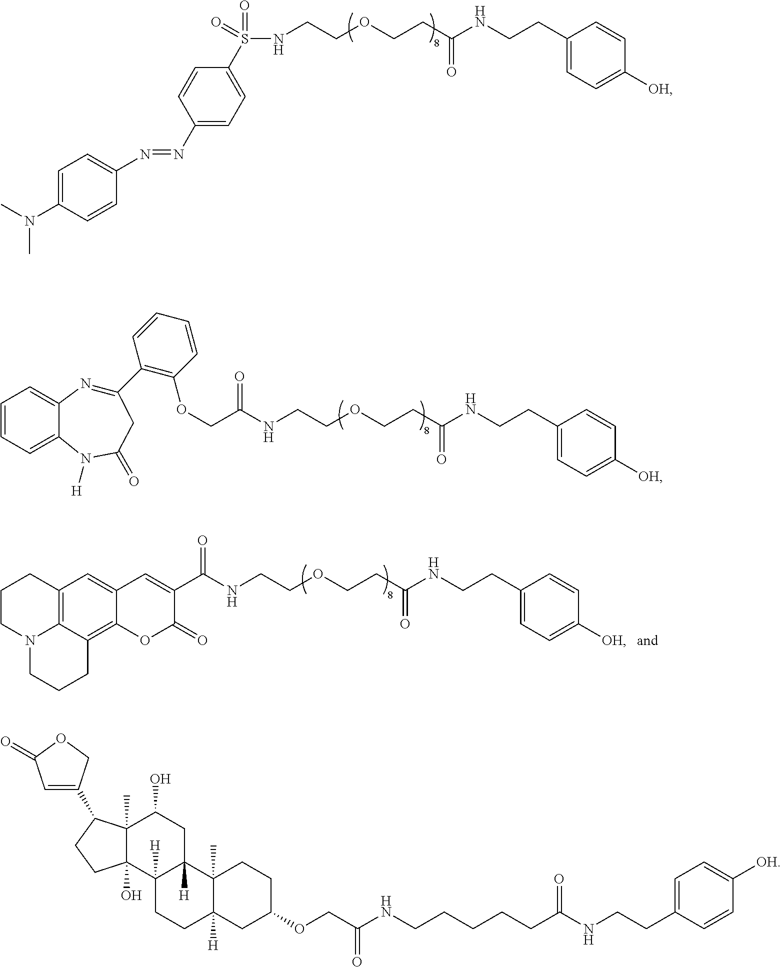

Embodiments of kits including a hapten conjugate as described above also are disclosed. In some embodiments, the hapten conjugate is a hapten-tyramide conjugate. In certain embodiments, the kit further includes a peroxide solution, such as a hydrogen peroxide solution. In a particular embodiment, the kit includes a hapten-tyramide conjugate having the formula:

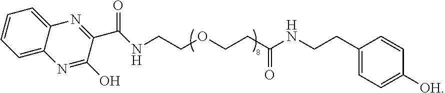

Embodiments of methods for using the hapten conjugates are disclosed. In general the method includes the steps of a) immobilizing a peroxidase on a target in a sample, wherein the peroxidase is capable of reacting with a peroxidase-activatable aryl moiety, e.g., tyramine or a tyramine derivative, b) contacting the sample with a solution comprising a hapten conjugate, wherein the hapten conjugate comprises a hapten bound to a peroxidase-activatable aryl moiety as described above, and c) contacting the sample with a solution comprising peroxide, whereby the hapten conjugate reacts with the peroxidase and the peroxide, forming a covalent bond to the immobilized peroxidase or proximal to the immobilized peroxidase; and d) locating the target in the sample by detecting the hapten.

In some embodiments, the peroxidase is horseradish peroxidase. In certain embodiments, the peroxidase is conjugated to a moiety—such as an antibody, nucleotide, oligonucleotide, protein, peptide, or amino acid—capable of binding directly or indirectly to the target.

In some embodiments, the target includes a nucleic acid sequence, and peroxidase is immobilized on the target by immobilizing a hapten-labeled probe on the sample, wherein the probe is capable of recognizing and binding to the target and comprises DNA, RNA, a locked nucleic acid oligomer, or an oligonucleotide; and contacting the sample with an antibody-peroxidase conjugate. In certain embodiments, the antibody-peroxidase conjugate includes an anti-hapten antibody capable of recognizing and binding to the hapten-labeled probe. In other embodiments, the sample is contacted with an anti-hapten antibody capable of recognizing and binding to the hapten-labeled probe before contacting the sample with an antibody-peroxidase conjugate including an antibody capable of recognizing and binding to the anti-hapten antibody.

The target may be located in the sample when the hapten is detected directly or indirectly (e.g., via a detectable label) by any suitable means. In some embodiments, the target is located by brightfield microscopy, fluorescence microscopy or spectroscopy, digital image analysis, or any combination thereof.

In some embodiments, the hapten is detected directly. For example, if the hapten is conjugated to a quantum dot, the quantum dot may be detected by its fluorescence at a characteristic wavelength. In other embodiments, detecting the hapten includes contacting the sample with an anti-hapten antibody and a detectable label, and detecting the label. In certain embodiments, the detectable label is conjugated to the anti-hapten antibody to form an anti-hapten antibody-label conjugate, and the conjugate binds to the hapten. In other embodiments, the sample is contacted with the anti-hapten antibody, which binds to the hapten. The sample then is contacted with an antibody conjugate capable of binding to the anti-hapten antibody, wherein the antibody conjugate includes the detectable label or a component of a detectable label system. In certain embodiments, the component of the detectable label system is an enzyme, such as horseradish peroxidase or alkaline phosphatase, which reacts with a chromogenic substrate or a substrate/chromogen complex thereby producing a detectable chromogenic deposition. In other embodiments, the label is a fluorescent label, such as a quantum dot.

In some embodiments, the method is suitable for detecting two or more targets in a sample. In general, the method includes the steps of a) providing a sample comprising two or more targets; b) immobilizing a first peroxidase on a first target in the sample; c) contacting the sample with a solution comprising a first hapten conjugate and a solution comprising peroxide, wherein the first hapten conjugate includes a first hapten bound to a peroxidase-activatable aryl moiety; d) immobilizing a subsequent peroxidase on a subsequent target in the sample; e) contacting the sample with a solution comprising a subsequent hapten conjugate and a solution comprising peroxide, wherein the subsequent hapten conjugate includes a subsequent hapten bound to a peroxidase-activatable aryl moiety, wherein the subsequent hapten is not the same as the first hapten or any other subsequent hapten; and f) locating the two or more targets in the sample by detecting the first and subsequent haptens. In some embodiments, the first peroxidase is inactivated before immobilizing the subsequent peroxidase on the subsequent target. In certain embodiments, the first hapten conjugate and the subsequent hapten conjugate are hapten-tyramide conjugates.

In some embodiments, the method is suitable for detecting two or more nucleic acid sequence targets in a sample. In general, the method includes the steps of a) providing a sample comprising two or more nucleic acid sequence targets; b) immobilizing a first probe comprising DNA, RNA, or an oligonucleotide on the sample, wherein the first probe is labeled with a first hapten and is capable of recognizing and binding to a first target; c) immobilizing a subsequent probe comprising DNA, RNA, or an oligonucleotide on the sample, wherein the subsequent probe is labeled with a subsequent hapten and is capable of recognizing and binding to a subsequent target, and wherein the subsequent hapten is not the same as the first hapten or any other subsequent hapten; d) contacting the sample with a first anti-hapten antibody-peroxidase conjugate, wherein the first anti-hapten antibody is capable of recognizing and binding to the first hapten; e) contacting the sample with a solution comprising a first hapten conjugate and a solution comprising peroxide, wherein the first hapten tyramide conjugate comprises the first hapten bound to a peroxidase-activatable aryl moiety; f) contacting the sample with a subsequent anti-hapten antibody-peroxidase conjugate, wherein the subsequent anti-hapten antibody is capable of binding and recognizing to the subsequent hapten; g) contacting the sample with a solution comprising a subsequent hapten conjugate and a solution comprising peroxide, wherein the subsequent hapten tyramide conjugate comprises the subsequent hapten bound to a peroxidase-activatable aryl moiety; and h) locating the two or more targets in the sample by detecting the first and subsequent haptens. In some embodiments, the first anti-hapten antibody-peroxidase conjugate is deactivated before contacting the sample with the subsequent anti-hapten-antibody conjugate. In certain embodiments, the first hapten conjugate and the subsequent hapten conjugate are hapten-tyramide conjugates.

In some embodiments, locating the two or more targets in the sample further includes contacting the sample with a solution comprising a first anti-hapten antibody-quantum dot conjugate comprising a first antibody capable of recognizing and binding to the first hapten and a first quantum dot, and a subsequent anti-hapten antibody-quantum dot conjugate comprising a subsequent antibody capable of recognizing and binding to the subsequent hapten and a subsequent quantum dot, wherein the subsequent quantum dot is not the same as the first quantum dot or any other subsequent quantum dot, and detecting fluorescence from the first and subsequent quantum dots.

In a particular embodiment, the sample is obtained from a subject suspected of having breast cancer, and at least one of the first probe or the subsequent probe is an anti-sense RNA probe capable of hybridizing to HER2 mRNA, ER mRNA, Ki67 mRNA, or PGR mRNA.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram of one embodiment of a method for using a hapten-tyramide conjugate.

FIG. 2 is a schematic diagram of one embodiment of a method for amplifying the signal from a hapten-tyramide conjugate.

FIG. 3A is a schematic diagram of one embodiment of a method for using a hapten-tyramide conjugate.

FIG. 3B is a schematic diagram of another embodiment of a method for using a hapten-tyramide conjugate.

FIG. 4 is a schematic diagram of an embodiment of a method for using hapten-tyramide conjugates in a multiplexed assay.

FIGS. 5A and 5B together are a schematic diagram of one embodiment of a method for using hapten-tyramide conjugates in a multiplexed mRNA-ISH assay.

FIG. 6 is a photomicrograph depicting the evaluation of bcl2 (124) antibody on tonsil tissue using a BD-tyramide conjugate diluted to 5.5 μM in 0.75 mM sodium stannate, 40 mM boric acid, 10 mM sodium tetraborate decahydrate, and 30 mM sodium chloride (tyramide amplification diluent).

FIG. 7 is a photomicrograph depicting the evaluation of bcl2 (124) antibody on tonsil tissue using a BD-tyramide conjugate diluted to 55 μM in tyramide amplification diluent.

FIG. 8 is a photomicrograph depicting the evaluation of bcl2 (124) antibody on tonsil tissue using a BF-tyramide conjugate diluted to 5.5 μM in tyramide amplification diluent.

FIG. 9 is a photomicrograph depicting the evaluation of bcl2 (124) antibody on tonsil tissue using a BF-tyramide conjugate diluted to 55 μM in tyramide amplification diluent.

FIG. 10 is a photomicrograph depicting the evaluation of bcl2 (124) antibody on tonsil tissue using a DABSYL-tyramide conjugate diluted to 5.5 μM in tyramide amplification diluent.

FIG. 11 is a photomicrograph depicting the evaluation of bcl2 (124) antibody on tonsil tissue using a DABSYL-tyramide conjugate diluted to 55 μM in tyramide amplification diluent.

FIG. 12 is a photomicrograph depicting the evaluation of bcl2 (124) antibody on tonsil tissue using a DCC-tyramide conjugate diluted to 5.5 μM in tyramide amplification diluent.

FIG. 13 is a photomicrograph depicting the evaluation of bcl2 (124) antibody on tonsil tissue using a DCC-tyramine conjugate diluted to 55 μM in tyramide amplification diluent.

FIG. 14 is a photomicrograph depicting the evaluation of bcl2 (124) antibody on tonsil tissue using a DIG-tyramide conjugate diluted to 5.5 μM in tyramide amplification diluent.

FIG. 15 is a photomicrograph depicting the evaluation of bcl2 (124) antibody on tonsil tissue using a DIG-tyramide conjugate diluted to 55 μM in tyramide amplification diluent.

FIG. 16 is a photomicrograph depicting the evaluation of bcl2 (124) antibody on tonsil tissue using a DNP-tyramide conjugate diluted to 5.5 μM in tyramide amplification diluent.

FIG. 17 is a photomicrograph depicting the evaluation of bcl2 (124) antibody on tonsil tissue using a DNP-tyramine conjugate diluted to 55 μM in tyramide amplification diluent.

FIG. 18 is a photomicrograph depicting the evaluation of bcl2 (124) antibody on tonsil tissue using a FITC-tyramide conjugate diluted to 5.5 μM in tyramide amplification diluent.

FIG. 19 is a photomicrograph depicting the evaluation of bcl2 (124) antibody on tonsil tissue using a FITC-tyramide conjugate diluted to 55 μM in tyramide amplification diluent.

FIG. 20 is a photomicrograph depicting the evaluation of bcl2 (124) antibody on tonsil tissue using a HQ-tyramide conjugate diluted to 5.5 μM in tyramide amplification diluent.

FIG. 21 is a photomicrograph depicting the evaluation of bcl2 (124) antibody on tonsil tissue using a HQ-tyramide conjugate diluted to 55 μM in tyramide amplification diluent.

FIG. 22 is a photomicrograph depicting the evaluation of bcl2 (124) antibody on tonsil tissue using a NCA-tyramide conjugate diluted to 5.5 μM in tyramide amplification diluent.

FIG. 23 is a photomicrograph depicting the evaluation of bcl2 (124) antibody on tonsil tissue using a NCA-tyramide conjugate diluted to 55 μM in tyramide amplification diluent.

FIG. 24 is a photomicrograph depicting the evaluation of bcl2 (124) antibody on tonsil tissue using a NP-tyramide conjugate diluted to 5.5 μM in tyramide amplification diluent.

FIG. 25 is a photomicrograph depicting the evaluation of bcl2 (124) antibody on tonsil tissue using a NP-tyramide conjugate diluted to 55 μM in tyramide amplification diluent.

FIG. 26 is a photomicrograph depicting the evaluation of bcl2 (124) antibody on tonsil tissue using a PPT-tyramide conjugate diluted to 5.5 μM in tyramide amplification diluent.

FIG. 27 is a photomicrograph depicting the evaluation of bcl2 (124) antibody on tonsil tissue using a PPT-tyramide conjugate diluted to 55 μM in tyramide amplification diluent.

FIG. 28 is a photomicrograph depicting the evaluation of bcl2 (124) antibody on tonsil tissue using a Rhod-tyramide conjugate diluted to 5.5 μM in tyramide amplification diluent.

FIG. 29 is a photomicrograph depicting the evaluation of bcl2 (124) antibody on tonsil tissue using a Rhod-tyramide conjugate diluted to 55 μM in tyramide amplification diluent.

FIG. 30 is a photomicrograph depicting the evaluation of bcl2 (124) antibody on tonsil tissue using a ROT-tyramide conjugate diluted to 5.5 μM in tyramide amplification diluent.

FIG. 31 is a photomicrograph depicting the evaluation of bcl2 (124) antibody on tonsil tissue using a ROT-tyramide conjugate diluted to 55 μM in tyramide amplification diluent.

FIG. 32 is a photomicrograph depicting the evaluation of bcl2 (124) antibody on tonsil tissue using a TS-tyramide conjugate diluted to 5.5 μM in tyramide amplification diluent.

FIG. 33 is a photomicrograph depicting the evaluation of bcl2 (124) antibody on tonsil tissue using a TS-tyramide conjugate diluted to 55 μM in tyramide amplification diluent.

FIG. 34 is a graph illustrating the signal intensity and range of native-hapten antibody detection efficiencies.

FIG. 35 is a fluorescent micrograph depicting the fluorescence of BD-labeled anti-sense and sense RNA probes as detected with a biotinylated goat anti-mouse polyclonal antibody and streptavidin conjugated to Qd655.

FIG. 36 is a fluorescent micrograph depicting the fluorescence of BF-labeled anti-sense and sense RNA probes as detected with a biotinylated goat anti-mouse polyclonal antibody and streptavidin conjugated to Qd655.

FIG. 37 is a fluorescent micrograph depicting the fluorescence of DABSYL-labeled anti-sense and sense RNA probes as detected with a biotinylated goat anti-mouse polyclonal antibody and streptavidin conjugated to Qd655.

FIG. 38 is a fluorescent micrograph depicting the fluorescence of DCC-labeled anti-sense and sense RNA probes as detected with a biotinylated goat anti-mouse polyclonal antibody and streptavidin conjugated to Qd655.

FIG. 39 is a fluorescent micrograph depicting the fluorescence of DIG-labeled anti-sense and sense RNA probes as detected with a biotinylated goat anti-mouse polyclonal antibody and streptavidin conjugated to Qd655.

FIG. 40 is a fluorescent micrograph depicting the fluorescence of DNP-labeled anti-sense and sense RNA probes as detected with a biotinylated goat anti-mouse polyclonal antibody and streptavidin conjugated to Qd655.

FIG. 41 is a fluorescent micrograph depicting the fluorescence of HQ-labeled anti-sense and sense RNA probes as detected with a biotinylated goat anti-mouse polyclonal antibody and streptavidin conjugated to Qd655.

FIG. 42 is a fluorescent micrograph depicting the fluorescence of NCA-labeled anti-sense and sense RNA probes as detected with a biotinylated goat anti-mouse polyclonal antibody and streptavidin conjugated to Qd655.

FIG. 43 is a fluorescent micrograph depicting the fluorescence of NP-labeled anti-sense and sense RNA probes as detected with a biotinylated goat anti-mouse polyclonal antibody and streptavidin conjugated to Qd655.

FIG. 44 is a fluorescent micrograph depicting the fluorescence of PPT-labeled anti-sense and sense RNA probes as detected with a biotinylated goat anti-mouse polyclonal antibody and streptavidin conjugated to Qd655.

FIG. 45 is a fluorescent micrograph depicting the fluorescence of ROT-labeled anti-sense and sense RNA probes as detected with a biotinylated goat anti-mouse polyclonal antibody and streptavidin conjugated to Qd655.

FIG. 46 is a fluorescent micrograph depicting the fluorescence of TS-labeled anti-sense and sense RNA probes as detected with a biotinylated goat anti-mouse polyclonal antibody and streptavidin conjugated to Qd655.

FIG. 47 is a graph depicting the relative signal intensity obtained with native anti-hapten antibodies and embodiments of the disclosed hapten-tyramide conjugates.

FIG. 48 is a series of fluorescent micrographs depicting the fluorescence of hapten-tyramide conjugates detected using cognate monoclonal antibodies followed by Qd655-conjugated goat anti-mouse polyclonal antibodies.

FIG. 49 is two fluorescent micrographs depicting the fluorescence of a DNP-tyramide conjugate detected with a cognate monoclonal antibody-Qd655 conjugate.

FIGS. 50A-D are fluorescent micrographs depicting the fluorescence of DNP-, BF-, NP-, and TS-labeled anti-sense 18S RNA probes hybridized to Calu-3 xenograft tissue as detected with anti-hapten monoclonal antibodies conjugated to Qd655, Qd605, Qd585, and Qd565, respectively.

FIG. 51A is a composite image of FIGS. 50A-D.

FIG. 51B is a composite image of fluorescent micrographs of DNP-, BF-, NP-, and TS-labeled sense-strand 18S RNA probes hybridized as detected with anti-hapten monoclonal antibodies conjugated to Qd655, Qd605, Qd585, and Qd565, respectively.

FIGS. 52A-D are fluorescent micrographs depicting the fluorescence of NP-labeled Ki67, TS-labeled HER2, BF-labeled ER, and DNP-labeled ACTB anti-sense RNA probes hybridized to Calu-3 xenograft tissue as detected with anti-hapten monoclonal antibodies conjugated to Qd525, Qd565, Qd605, and Qd655, respectively.

FIGS. 53A-D are fluorescent micrographs depicting the fluorescence of NP-labeled Ki67, TS-labeled HER2, BF-labeled ER, and DNP-labeled ACTB anti-sense RNA probes hybridized to MCF-7 xenograft tissue as detected with anti-hapten monoclonal antibodies conjugated to Qd525, Qd565, Qd605, and Qd655, respectively.

FIG. 54A is a composite image of FIGS. 52A-D.

FIG. 54B is a composite image of FIGS. 53A-D.

FIGS. 55A-C are fluorescent micrographs of DNP-labled HER2 antisense RNA probes hybridized to Calu-3, ZR75-1, and MCF-7 xenograft tissues, respectively, and detected with anti-hapten monoclonal antibodies conjugated to Qd655.

FIG. 56 is a graph depicting the HER2:ACTB mRNA ratios in Calu-3, ZR75-1, and MCF-7 xenograft tissues as detected by qPCR and mRNA-ISH assays.

FIG. 57 is a fluorescent micrograph showing stochastic expression of HER2 in Calu-3 xenograft cells. Expression was visualized using a DNP-labled HER2 antisense RNA probe hybridized to the Calu-3 xenograft tissue, and detected with anti-hapten monoclonal antibodies conjugated to Qd655.

FIG. 58 is a photomicrograph depicting the evaluation of an miRNA LNA (locked nucleic acid) probe, miR205, on lobular breast cancer tissue without amplification.

FIG. 59 is a photomicrograph depicting the evaluation of miR205 on lobular breast cancer tissue with amplification using an HQ-tyramide conjugate.

FIG. 60 is a photomicrograph depicting the evaluation of an miRNA LNA probe, miR126, on tonsil tissue without amplification.

FIG. 61 is a photomicrograph depicting the evaluation of miR126 on tonsil tissue with amplification using an HQ-tyramide conjugate.

### DETAILED DESCRIPTION

### I. Terms and Abbreviations

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all nucleotide sizes or amino acid sizes, and all molecular weight or molecular mass values, given for nucleic acids or polypeptides or other compounds are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various examples of this disclosure, the following explanations of specific terms are provided:

ACTB: Beta-actin.

Amplification: Amplification refers to the act or result of making a signal stronger.

Antibody: “Antibody” collectively refers to immunoglobulins or immunoglobulin-like molecules (including by way of example and without limitation, IgA, IgD, IgE, IgG and IgM, combinations thereof, and similar molecules produced during an immune response in any vertebrate, for example, in mammals such as humans, goats, rabbits and mice) and antibody fragments that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample.

More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

This includes intact immunoglobulins and the variants and portions of them well known in the art. Antibody fragments include proteolytic antibody fragments [such as F(ab′)₂ fragments, Fab′ fragments, Fab′-SH fragments and Fab fragments as are known in the art], recombinant antibody fragments (such as sFv fragments, dsFv fragments, bispecific sFv fragments, bispecific dsFv fragments, F(ab)′₂ fragments, single chain Fv proteins (“scFv”), disulfide stabilized Fv proteins (“dsFv”), diabodies, and triabodies (as are known in the art), and camelid antibodies (see, for example, U.S. Pat. Nos. 6,015,695; 6,005,079, 5,874,541; 5,840,526; 5,800,988; and 5,759,808).

Antigen: A compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids and proteins.

BD: Benzodiazepine, e.g., (E)-2-(2-(2-oxo-2,3-dihydro-1H-benzo[b][1,4]diazepin-4-yl)phenoxy)acetaminde, a hapten.

BF: Benzofurazan, e.g., 2,1,3-benzoxadiazole-5-carbamide, a hapten.

Conjugating, joining, bonding or linking: Joining one molecule to another molecule to make a larger molecule. For example, making two polypeptides into one contiguous polypeptide molecule, or covalently attaching a hapten or other molecule to a polypeptide, such as an scFv antibody.

Conjugate: A compound formed by the union of two or more compounds, e.g., an ester formed from an alcohol and an organic acid with elimination of water. Examples of conjugates include, but are not limited to, hapten-antibody conjugates, enzyme-antibody conjugates, hapten-tyramide conjugates, hapten-linker-tyramine conjugates, labeled probes (e.g., dinitrophenyl-labeled mRNA probes).

Coupled: The term “coupled” means joined together, either directly or indirectly. A first atom or molecule can be directly coupled or indirectly coupled to a second atom or molecule.

DABSYL: 4-(dimethylamino)azobenzene-4′-sulfonamide, a hapten.

DCC: 7-(diethylamino)coumarin-3-carboxylic acid (7-(diethylamino)-2-oxo-2H-chromene-3-carboxylic acid), a hapten.

Derivative: A derivative is a compound that is derived from a similar compound by replacing one atom or group of atoms with another atom or group of atoms.

Detectable Label: A detectable compound or composition that is attached directly or indirectly to another molecule, such as an antibody or a protein, to facilitate detection of that molecule. Specific, non-limiting examples of labels include fluorescent tags, enzymes, and radioactive isotopes.

DIG: Digoxigenin, a hapten.

DNP: 2,4-dinitrophenyl, a hapten.

Epitope: An antigenic determinant. These are particular chemical groups or contiguous or non-contiguous peptide sequences on a molecule that are antigenic, that is, that elicit a specific immune response. An antibody binds a particular antigenic epitope.

ER: Estrogen receptor; ER-positive breast cancers may benefit from anti-estrogen therapy.

FITC: Fluorescein isothiocyanate, a hapten.

Functional group: A specific group of atoms within a molecule that is responsible for the characteristic chemical reactions of the molecule. Exemplary functional groups include, without limitation, alkane, alkene, alkyne, arene, halo (fluoro, chloro, bromo, iodo), epoxide, hydroxyl, carbonyl (ketone), aldehyde, carbonate ester, carboxylate, ether, ester, peroxy, hydroperoxy, carboxamide, amine (primary, secondary, tertiary), ammonium, imide, azide, cyanate, isocyanate, thiocyanate, nitrate, nitrite, nitrile, nitroalkane, nitroso, pyridyl, phosphate, sulfonyl, sulfide, thiol (sulfhydryl), disulfide.

Hapten: A molecule, typically a small molecule, that can combine specifically with an antibody, but typically is substantially incapable of being immunogenic on its own.

HER2: Human epidermal growth factor receptor 2, a protein linked with higher aggressiveness in breast cancers.

Ki67: A protein encoded by the MKI67 gene; a nuclear protein associated with cellular proliferation and ribosomal RNA transcription.

Linker: As used herein, a linker is a molecule or group of atoms positioned between two moieties. For example, a hapten-tyramide conjugate may include a linker between the hapten and the tyramine or tyramine derivative. Typically, linkers are bifunctional, i.e., the linker includes a functional group at each end, wherein the functional groups are used to couple the linker to the two moieties. The two functional groups may be the same, i.e., a homobifunctional linker, or different, i.e., a heterobifunctional linker.

Locked nucleic acid (LNA): An LNA, often referred to as inaccessible RNA is a modified RNA nucleotide. The ribose moiety is modified with an extra bridge connecting the 2′ oxygen and 4′ carbon. LNA oligomers are commercially available, and are used to increase hybridization properties (e.g., melting temperature) of oligonucleotide probes.

Moiety: A moiety is a fragment of a molecule, or a portion of a conjugate.

Molecule of interest or Target: A molecule for which the presence, location and/or concentration is to be determined. Examples of molecules of interest include proteins and nucleic acid sequences.

Monoclonal antibody: An antibody produced by a single clone of B-lymphocytes or by a cell into which the light and heavy chain genes of a single antibody have been transfected. Monoclonal antibodies are produced by methods known to those of skill in the art. Monoclonal antibodies include humanized monoclonal antibodies.

Multiplex, -ed, -ing: Embodiments of the present invention allow multiple targets in a sample to be detected substantially simultaneously, or sequentially, as desired, using plural different conjugates. Multiplexing can include identifying and/or quantifying nucleic acids generally, DNA, RNA, peptides, proteins, both individually and in any and all combinations. Multiplexing also can include detecting two or more of a gene, a messenger and a protein in a cell in its anatomic context.

NCA: Nitrocinnamic acid, e.g., 4,5-dimethoxy-2-nitrocinnamide, a hapten.

NP: Nitropyrazole, e.g., 5-nitro-3-pyrazolecarbamide, a hapten.

Peroxidase-activatable aryl moiety: An aryl moiety capable of forming a free radical when combined with a peroxidase enzyme and peroxide. Typically, the peroxidase-activatable aryl moiety has a general formula

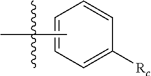

where R_{C} is a functional group capable of forming a free radical when combined with a peroxidase enzyme and peroxide. Suitable functional groups include hydroxyl, ether, amine, and substituted amine groups.

PGR or PR: Progesterone receptor; growth of PGR-positive cancer cells is influenced by progesterone.

Polypeptide: A polymer in which the monomers are amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence, and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

PPT: Podophyllotoxin, e.g., p-methoxyphenylpyrazopodophyllamide, a hapten.

Protein: A molecule, particularly a polypeptide, comprised of amino acids.

Proximal: The term “proximal” means being situated at or near the point of attachment or origin. As used herein, proximal means within about 100 nm, within about 50 nm, within about 10 nm, or within about 5 nm of a peroxidase conjugate immobilized on a target within a sample. Proximal also may indicate within a range of about 10 angstroms to about 100 nm, about 10 angstroms to about 50 nm, about 10 angstroms to about 10 nm, or about 10 angstroms to about 5 nm.

Purified: The term “purified” does not require absolute purity; rather, it is intended as a relative term. Thus, for example, a purified peptide, protein, conjugate, or other active compound is one that is isolated in whole or in part from proteins or other contaminants. Generally, substantially purified peptides, proteins, conjugates, or other active compounds for use within the disclosure comprise more than 80% of all macromolecular species present in a preparation prior to admixture or formulation of the peptide, protein, conjugate or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, conjugate or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

Quantum dot: A nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. A variety of quantum dots having various surface chemistries and fluorescence characteristics are commercially available from Invitrogen Corporation, Eugene, Oreg. (see, for example, U.S. Pat. Nos. 6,815,064, 6,682,596 and 6,649,138, each of which patents is incorporated by reference herein). Quantum dots are also commercially available from Evident Technologies (Troy, N.Y.). Other quantum dots include alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN quantum dots (Alloy quantum dots and methods for making the same are disclosed, for example, in US Application Publication No. 2005/0012182 and PCT Publication WO 2005/001889).

Reactive Groups: Formulas throughout this application refer to “reactive groups,” which can be any of a variety of groups suitable for coupling a first unit to a second unit as described herein. For example, the reactive group might be an amine-reactive group, such as an isothiocyanate, an isocyanate, an acyl azide, an NHS ester, an acid chloride, such as sulfonyl chloride, aldehydes and glyoxals, epoxides and oxiranes, carbonates, arylating agents, imidoesters, carbodiimides, anhydrides, and combinations thereof. Suitable thiol-reactive functional groups include haloacetyl and alkyl halides, maleimides, aziridines, acryloyl derivatives, arylating agents, thiol-disulfide exchange reagents, such as pyridyl disulfides, TNB-thiol, and disulfide reductants, and combinations thereof. Suitable carboxylate-reactive functional groups include diazoalkanes, diazoacetyl compounds, carbonyldiimidazole compounds, and carbodiimides. Suitable hydroxyl-reactive functional groups include epoxides and oxiranes, carbonyldiimidazole, N,N′-disuccinimidyl carbonates or N-hydroxysuccinimidyl chloroformates, periodate oxidizing compounds, enzymatic oxidation, alkyl halogens, and isocyanates. Aldehyde and ketone-reactive functional groups include hydrazines, Schiff bases, reductive amination products, Mannich condensation products, and combinations thereof. Active hydrogen-reactive compounds include diazonium derivatives, Mannich condensation products, iodination reaction products, and combinations thereof. Photoreactive chemical functional groups include aryl azides, halogenated aryl azides, benzophonones, diazo compounds, diazirine derivatives, and combinations thereof.

Rhod: Rhodamine, a hapten. One example of a rhodamine hapten has the following chemical structure.

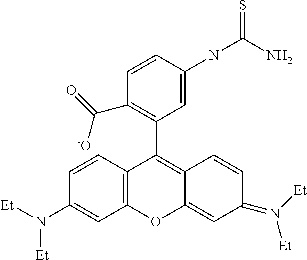

ROT: Rotenone, e.g., rotenone isoxazoline, a hapten.

Sample: A biological specimen containing genomic DNA, RNA (including mRNA), protein, or combinations thereof, obtained from a subject. Examples include, but are not limited to, peripheral blood, urine, saliva, tissue biopsy, surgical specimen, amniocentesis samples and autopsy material.

Specific binding moiety: A member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A), nucleic acid sequences, and protein-nucleic acids. Specific binding moieties can also include the molecules (or portions thereof) that are specifically bound by such specific binding proteins.

TS: Thiazolesulfonamide, e.g., 2-acetamido-4-methyl-5-thiazolesulfonamide, a hapten.

### II. Haptens

Disclosed embodiments of haptens include pyrazoles, particularly nitropyrazoles; nitrophenyl compounds; benzofurazans; triterpenes; ureas and thioureas, particularly phenyl ureas, and even more particularly phenyl thioureas; rotenone and rotenone derivatives, also referred to herein as rotenoids; oxazole and thiazoles, particularly oxazole and thiazole sulfonamides; coumarin and coumarin derivatives; cyclolignans, exemplified by Podophyllotoxin and Podophyllotoxin derivatives; and combinations thereof. Embodiments of haptens and methods for their preparation and use are disclosed in U.S. Pat. No. 7,695,929, which is incorporated in its entirety herein by reference.

For the general formulas provided below, if no substituent is indicated, a person of ordinary skill in the art will appreciate that the substituent is hydrogen. A bond that is not connected to an atom, but is shown, for example, extending to the interior of a ring system, indicates that the position of such substituent is variable. A curved line drawn through a bond indicates that some additional structure is bonded to that position, typically a linker or the functional group or moiety used to couple the hapten to a tyramine or tyramine derivative. Moreover, if no stereochemistry is indicated for compounds having one or more chiral centers, all enantiomers and diasteromers are included. Similarly, for a recitation of aliphatic or alkyl groups, all structural isomers thereof also are included. Unless otherwise stated, R groups in the general formulas provided below independently are selected from: hydrogen, acyl, aldehyde, alkoxy, aliphatic, particularly lower aliphatic (e.g., isoprene), substituted aliphatic, heteroaliphatic, e.g., organic chains having heteroatoms, such as oxygen, nitrogen, sulfur, alkyl, particularly alkyl having 20 or fewer carbon atoms, and even more typically lower alkyl having 10 or fewer atoms, such as methyl, ethyl, propyl, isopropyl, and butyl, substituted alkyl, such as alkyl halide (e.g. —CX₃ where X is a halide, and combinations thereof, either in the chain or bonded thereto,), oxime, oxime ether (e.g., methoxyimine, CH₃—O—N═) alcohols (i.e. aliphatic or alkyl hydroxyl, particularly lower alkyl hydroxyl) amido, amino, amino acid, aryl, alkyl aryl, such as benzyl, carbohydrate, monosaccharides, such as glucose and fructose, disaccharides, such as sucrose and lactose, oligosaccharides and polysaccharides, carbonyl, carboxyl, carboxylate (including salts thereof, such as Group I metal or ammonium ion carboxylates), cyclic, cyano (—CN), ester, such as alkyl ester, ether, exomethylene, halogen, heteroaryl, heterocyclic, hydroxyl, hydroxylamine, oxime (HO—N═), keto, such as aliphatic ketones, nitro, sulfhydryl, sulfonyl, sulfoxide, exomethylene and combinations thereof.

1. Azoles

A first general class of haptens of the present invention is azoles, typically oxazoles and pyrazoles, more typically nitro oxazoles and nitro pyrazoles, having the following general chemical formula.

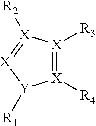

R₁-R₄ can be any group that does not interfere with, and potentially facilitates, the function as a hapten. More specifically, R₁-R₄ are defined as above. Two or more of these R₁-R₄ substituents also may be atoms, typically carbon atoms, in a ring system bonded or fused to the compounds having the illustrated general formula. At least one of the R₁-R₄ substituents is bonded to a linker or is a functional group suitable for coupling to a linker or a tyramine or tyramine derivative. R₁-R₄ most typically are aliphatic, hydrogen or nitro groups, even more typically alkyl, hydrogen or nitro, and still even more typically lower (10 or fewer carbon atoms) alkyl, hydrogen, nitro, or combinations thereof. The number of nitro groups can vary, but most typically there are 1 or 2 nitro groups. X independently is nitrogen or carbon. Y is oxygen, sulfur or nitrogen. If Y is oxygen or sulfur, then there is no R₁ group. If Y is nitrogen, then there is at least one R₁ group.

A person of ordinary skill in the art will appreciate that, for compounds having 2 or more heteroatoms, the relative positions thereof are variable. Moreover, more than two heteroatoms also are possible, such as with triazines.

At least one of R₁-R₄ for these azole compounds is bonded to some other group or is a variable functional group. For example, the illustrated compounds can be coupled either directly to a tyramine or tyramine derivative or to a linker at any of the suitable positions about the azole ring.

Working embodiments typically were mono- or dinitro pyrazole derivatives, such that at least one of R₁-R₄ is a nitro group, with the remaining R₁-R₄ being used to couple the hapten to a linker or a tyramine or tyramine derivative.

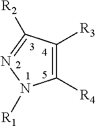

One particular compound had the following structure.

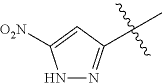 [SMILES: *c1cc([N+](=O)[O-])[nH]n1]

2. Nitroaryl

A second general class of haptens of the present invention are nitroaryl compounds. Exemplary nitroaryl compounds include, without limitation, nitrophenyl, nitrobiphenyl, nitrotriphenyl, etc., and any and all heteroaryl counterparts, having the following general chemical formula.

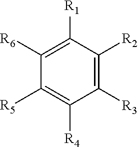

With reference to this general formula, at least one of R₁-R₆ is nitro. If more than one of R₁-R₆ is nitro, all combinations of relative ring positions of plural nitro substituents, or nitro substituents relative to other ring substituents, are included within this class of disclosed haptens. Dinitroaryl compounds are most typical. The remaining ring substituents are defined as above. At least one of the R₁-R₆ substituents is bonded to a linker or is a functional group suitable for coupling to a linker or a tyramine or tyramine derivative.

Two or more of the R₁-R₆ substituents also may be atoms, typically carbon atoms, in a ring system, such as naphthalene (shown below) or anthracene type derivatives. Ring systems other than 6-membered ring systems can be formed, such as fused 6-5 ring systems.

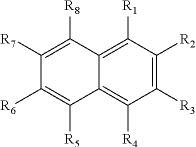

Again, at least one of the ring positions occupied by R₁-R₈ is bonded to a linker or is a variable functional group suitable for coupling, such as by covalent bonding, to a tyramine or tyramine derivative. For example, nitroaryl compounds of the present invention can include a functional group for coupling to a tyramine or tyramine derivative, or to a linker, at various optional ring locations.

Working embodiments are exemplified by nitrophenyl compounds. Solely by way of example, mononitroaryl compounds are exemplified by nitrocinnamide compounds. One embodiment of a nitrocinnamide-based compound is exemplified by 4,5-dimethoxy-2-nitrocinnamide, shown below.

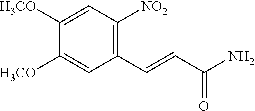 [SMILES: COc1cc(/C=C/C(N)=O)c([N+](=O)[O-])cc1OC]

The nitrophenyl class of compounds also is represented by dinitrophenyl compounds. At least one of the remaining carbon atoms of the ring positions not having a nitro group is bonded to a functional group, to a linker, or directly to a tyramine or tyramine derivative. Any and all combinations of relative positions of these groups are included within the class of disclosed haptens.

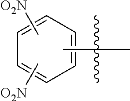

Working embodiments are more particularly exemplified by 2,4-dinitrophenyl compounds coupled to a linker, as illustrated below.

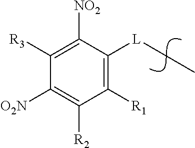

R₁-R₃ are as stated above.

3. Benzofurazans

Benzofurazans and derivatives thereof are another class of haptens within the scope of the present invention. A general formula for the benzofurazan-type compounds is provided below.

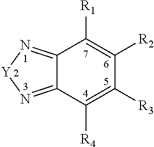

R₁-R₄ are defined as above. Two or more of these R₁-R₄ substituents also may be atoms, typically carbon atoms, in a ring system bonded or fused to the compounds having the illustrated general formula. At least one of the R₁-R₄ substituents is bonded to a linker or directly to a tyramine or tyramine derivative. Y is a carbon atom having R₅ and R₆ substituents, where R₅ and R₆ are as stated for R₁-R₄, oxygen or sulfur, typically oxygen.

Compounds where Y is oxygen are more particularly exemplified by compounds having the following structure, where R₁-R₄ are as stated above, and most typically are independently hydrogen and lower alkyl.

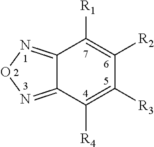

One working embodiment of a compound according to this class of haptens had the following chemical structure.

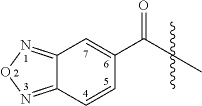 [SMILES: *C(=O)c1ccc2nonc2c1]

4. Triterpenes

Triterpenes are another class of haptens within the scope of the present invention. The basic ring structure common to the cyclic triterpenes has four six-membered fused rings, A-D, as indicated below.

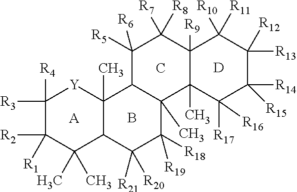

A number of publications discuss naturally occurring, semi-synthetic and synthetic triterpene species within the genus of triterpenes useful for practicing the present invention, including: J. C. Connolly and R. A. Hill, Triterpenoids, Nat. Prod. Rep., 19, 494-513 (2002); Baglin et al., A Review of Natural and Modified Beculinic, Ursolic and Echinocystic Acid Derivatives as Potential Antitumor and Anti-HIV Agents, Mini Reviews in Medicinal Chemistry, 3, 525-539; W. N. and M. C. Setzer, Plant-Derived Triterpenoids as Potential Antineoplastic Agents, Mini Reviews in Medicinal Chemistry, 3, 540-556 (2003); and Baltina, Chemical Modification of Glycyrrhizic Acid as a Route to New Bioactive Compounds for Medicine, Current Medicinal Chemistry, 10, 155-171 92003); each of which is incorporated herein by reference.

Based on the present disclosure and working embodiments thereof, as well as disclosures provided by these prior publications, and with reference to this first general formula, R₁-R₂₁ are defined as above. Two or more of these R₁-R₂₁ substituents also may be atoms, typically carbon atoms, in a ring system bonded or fused to the compounds having the illustrated general formula. At least one of the R₁-R₂₁ substituents is bonded to a linker or is a functional group suitable for coupling to a linker or a tyramine or tyramine derivative. Y is a bond, thereby defining a 5-membered ring, or is a carbon atom bearing R₂₂ and R₂₃ substituents, where these R groups are as stated above.

Disclosed embodiments of triterpenes exemplifying this class of haptens also may include an E ring, and this E ring can be of various ring sizes, particularly rings having 5-7 atoms, typically carbon atoms, in the ring. For example, the E ring might be a 6-membered ring, as indicated by the following general formula, where R₁-R₃₁ are as stated above for R₁-R₂₁.

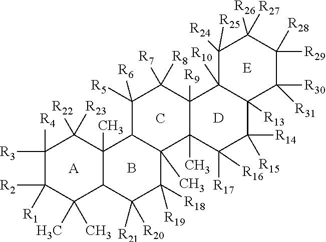

The following general formula indicates that the R₁₃ substituent may be an acyl group bearing an R₃₃ substituent selected from hydrogen, hydroxyl, ester, i.e.—OR₃₄ where R₃₄ is aliphatic, typically alkyl or substituted alkyl, and even more typically lower alkyl, amido, including primary amide (—NH₂), secondary amide (—NHR₃₅) and tertiary amide (—NR₃₅R₃₆), where R₃₅ and R₃₆ are aliphatic, typically lower aliphatic, more typically alkyl, substituted alkyl, and even more typically lower alkyl or substituted lower alkyl. This general formula also indicates that the R₁ substituent often is an OR₃₂ substituent, where R₃₂ is hydrogen or aliphatic, more typically alkyl or substituted alkyl, and even more typically lower alkyl. The remaining R groups are as stated above with reference to the first general formula.

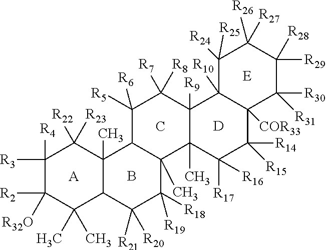

The E ring also may be a 5 membered ring, as indicated by the formula below where the R₁-R₂₉ groups are as stated above for R₁-R₂₁.

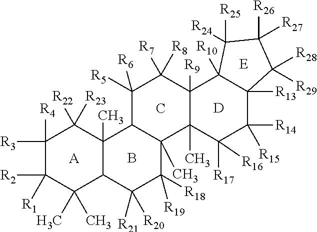

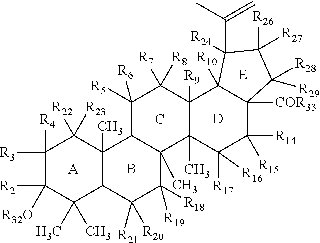

With reference to these general formulae, the R₁-R₂₉ groups are as stated above for R₁-R₂₁.

As with exemplary compounds where the E ring is a 6-membered ring, compounds where the E ring is a 5-membered ring also can include substituents at R₁ and R₁₃ as discussed above. Specifically, this general formula indicates that the R₁₃ substituent may be an acyl group bearing an R₃₃ substituent selected from hydrogen, hydroxyl, ester, i.e.—OR₃₄ where R₃₄ is aliphatic, typically alkyl or substituted alkyl, and even more typically lower alkyl, amido, including primary amide (—NH₂), secondary amide (—NHR₃₅) and tertiary amide (—NR₃₅R₃₆), where R₃₅ and R₃₆ are aliphatic, typically lower aliphatic, more typically alkyl, substituted alkyl, and even more typically lower alkyl or substituted lower alkyl. This general formula also indicates that the R₁ substituent often is an OR₃₂ substituent, where R₃₂ is hydrogen or aliphatic, more typically alkyl or substituted alkyl, and even more typically lower alkyl.

Exemplary compounds also include 5-membered rings as both the A and the E ring. General formulae for such exemplary compounds are provided below, where the R₁-R₂₉ substituents are as stated above.

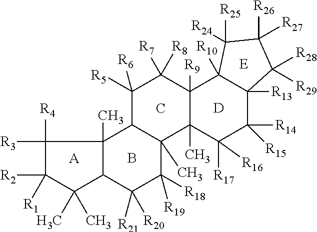

Again, the R₁ and R₁₃ substituents can be oxygen-based functional groups. The R₁₃ substituent may be an acyl group bearing an R₃₃ substituent selected from hydrogen, hydroxyl, ester, i.e.—OR₃₄ where R₃₄ is aliphatic, typically alkyl or substituted alkyl, and even more typically lower alkyl, amido, including primary amide (—NH₂), secondary amide (—NHR₃₅) and tertiary amide (—NR₃₅R₃₆), where R₃₅ and R₃₆ are aliphatic, typically lower aliphatic, more typically alkyl, substituted alkyl, and even more typically lower alkyl or substituted lower alkyl. This general formula also indicates that the R₁ substituent often is an OR₃₂ substituent, where R₃₂ is hydrogen or aliphatic, more typically alkyl or substituted alkyl, and even more typically lower alkyl.

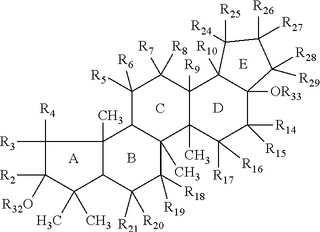

Exemplary triterpenes of the present invention also may include one or more sites of unsaturation in one or more of the A-E rings. Exemplary compounds often have at least one site of unsaturation in the C ring, such as the double bond in the C ring as indicated below.

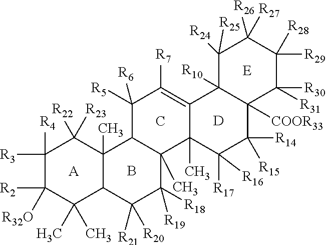

The site of unsaturation may be an alpha, beta unsaturated ketone, such as illustrated below for the C ring.

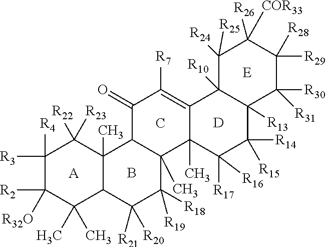

The triterpenes also have a number of stereogenic carbon atoms. A person of ordinary skill in the art will appreciate that particular enantiomers are most likely to occur naturally. While the naturally occurring enantiomer may be most available, and/or effective, for practicing disclosed embodiments, all other possible stereoisomers are within the scope of the present invention. Moreover, other naturally occurring triterpenes, or synthetic derivatives thereof, or fully synthetic compounds, may have (1) different stereochemistry, (2) different substituents, and further may be substituted at positions that are not substituted in the naturally occurring compounds. The general formulae provided above do not indicate stereochemistry at the chiral centers. This is to signify that both enantiomers at each chiral center, and all diastereomeric isomer combinations thereof, are within the scope of the present invention.

Particular working embodiments of the present invention are exemplified by the following general formula, in which the substituents are as stated above.

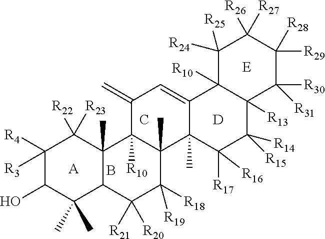

The stereochemistry and substituents for a naturally occurring triterpene useful as a hapten for practicing the present invention are shown below.

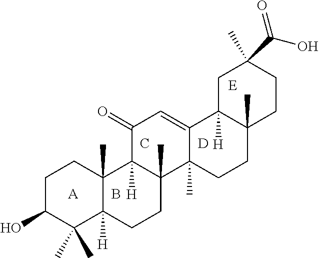

The hydroxyl group in the A ring typically is oxidized to a carbonyl functional group in working embodiments. As a result, the carbon atom bearing the carbonyl group is no longer a chiral center.

5. Ureas and Thioureas

Ureas and thioureas, particularly aryl and heteroaryl ureas and thioureas, are another class of haptens within the scope of the present invention. A general formula for urea-based haptens of the present invention is provided below.

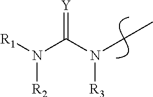

With reference to this general formula, R₁-R₃ are independently hydrogen, aliphatic, substituted aliphatic, typically alkyl, substituted alkyl, and even more typically lower alkyl and substituted lower alkyl, cyclic, heterocyclic, aryl and heteroaryl. More specifically, R₁ typically is aryl or aliphatic, often having at least one site of unsaturation to facilitate chromophoric activity. R₂ and R₃ most typically are independently hydrogen and lower alkyl. Y is oxygen (urea derivatives) or sulfur (thioureas).

Aryl derivatives typically have the following formula.

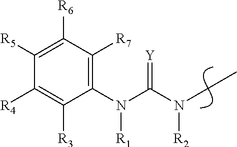

R₁-R₇ are as defined above. At least one of the R₃-R₇ substituents also is bonded to a linker or to a tyramine or tyramine derivative. Two or more of these R₃-R₇ substituents available for such bonding also may be atoms, typically carbon atoms, in a ring system bonded or fused to the compounds having the illustrated general formula.

Additional rings also can be present, as indicated by the exemplary structures provided below. The R groups are as stated above for R₁-R₇ and Y is oxygen or sulfur.

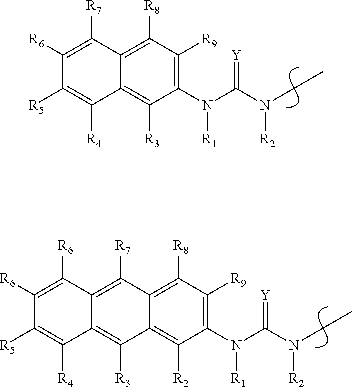

A particular subclass of thioureas is represented below.

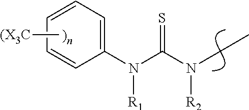

With reference to this general formula, n is 1 to 5, typically 1-2, R₁ and R₂ are independently hydrogen or lower alkyl, and X independently is a halide or combinations of different halides.

One example of a working embodiment of a phenyl thiourea is provided below.

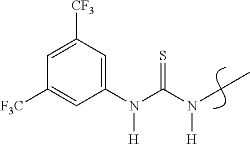 [SMILES: CCNC(=S)Nc1cc(C(F)(F)F)cc(C(F)(F)F)c1]

The trifluoromethyl groups are shown in the 2 and 4 positions relative to the thiourea moiety. A person of ordinary skill in the art will appreciate that compounds having all relative positions for disubstituted compounds, such as 2,3, and compounds having more than two trihaloalkyl substituents, at all possible relative positions of such plural trihaloalkyl substituents, also are within the scope of the present invention.

A particular example of a rhodamine thiourea hapten has the following formula.

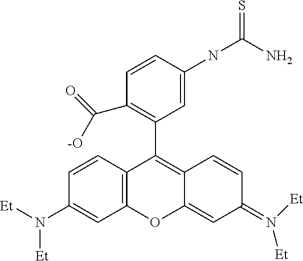

6. Rotenoids

Rotenone and rotenone-based haptens, collectively referred to as rotenoids, provide another class of haptens within the scope of the present invention. A first general formula for rotenone, and rotenone-based haptens, is provided below.

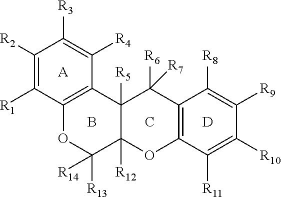

A number of publications discuss naturally occurring, semi-synthetic and synthetic rotenoids that are useful for describing the genus of rotenoids useful for practicing the present invention, including: Leslie Crombie and Donald Whiting, Biosynthesis in the Rotenoids Group of Natural Products: Application of Isotope Methodology, Phytochemistry, 49, 1479-1507 (1998); and Nianbai Fang, and John Casida, Cube Resin Insecticide: Identification and Biological Activity of 29 Rotenoid Constituents; each of which is incorporated herein by reference. Based on the present disclosure and working embodiments, as well as disclosures provided by these prior publications, and with reference to this first general formula, R₁-R₁₄ are defined as above. Two or more of these R₁-R₁₄ substituents also may be atoms, typically carbon atoms, in a ring system bonded or fused to the compounds having the illustrated general formula. At least one of the R₁-R₁₄ substituents also is bonded to a linker or to a tyramine or tyramine derivative.

While R₆ and R₇ can be as stated above, such substituents more typically independently are hydrogen, OR₁₅, where R₁₅ is hydrogen, aliphatic, substituted aliphatic, typically alkyl, substituted alkyl, and even more typically lower alkyl and substituted lower alkyl, such as lower alkyl halides, cyclic, heterocyclic, aryl and heteroaryl, —NR₂₁, where R₂₁ is hydrogen, aliphatic, substituted aliphatic, typically alkyl, substituted alkyl, and even more typically lower alkyl and substituted lower alkyl, such as lower alkyl halides, cyclic, heterocyclic, aryl and heteroaryl, or N-L-RG, where L is a linker or a reactive group, such as an amine, as discussed in more detail herein.

R₆ and R₇ also can form a double bond, such as a double bond to an oxygen to form a carbonyl. If R₆ and/or R₇ are not -L-RG, then at least one of the R substituents is bonded to a linker or to a tyramine or tyramine derivative.

The B ring also can include at least one additional site of unsaturation. For example, R₅ and R₁₂ can form a double bond.

R₁₀ and R₁₁ can be joined in a 5- or 6-membered ring. For example, R₁₀ and R₁₁ may define a pyran or furan ring, and more particularly is a substituted and/or unsaturated pyran or furan ring.

Certain exemplary rotenone-based haptens of the present invention also typically satisfy the following second general formula.

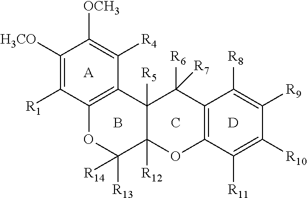

With reference to this second general formula, the R substituents are as stated above. If R₆ or R₇ is not -L-RG, then at least one of the remaining R groups is bonded to a linker or to a tyramine or tyramine derivative.

R₁₀ and R₁₁ can be joined in a 5- or 6-membered ring, such as a pyran or furan, and more particularly a substituted and/or unsaturated pyran or furan ring. Thus, a third general formula useful for describing certain rotenone-based haptens of the present invention is provided below, where the R substituents are as stated above.

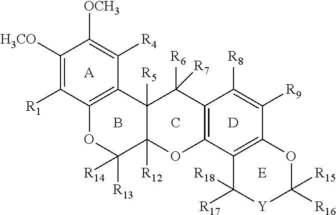

Y is a bond, thereby defining a 5-membered ring, or is a carbon atom in a 6-membered ring bearing R₁₉ and R₂₀ substituents, as shown below, where the R substituents are as stated above.

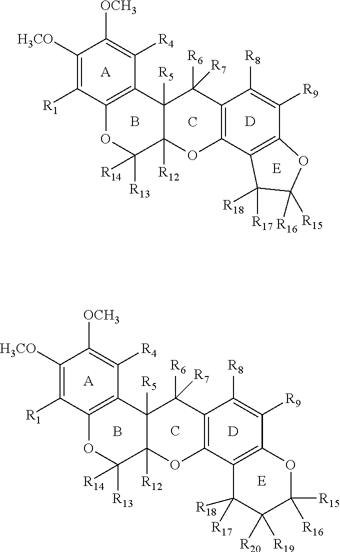

R₅ and R₁₂ at the ring juncture are shown without indicating particular stereochemistry. The naturally occurring compound has a cis-ring juncture, but racemic mixtures also are useful for practicing the present invention. Also, the trans stereoisomer likely quickly equilibrates to form the racemic mixture.

Working embodiments of compounds within this class more typically satisfy the following third general formula.

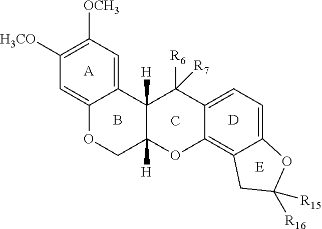

With reference to this general formula, R₆ and R₇ are hydrogen, alkyl, or define a double bond, such as to oxygen to form a carbonyl. R₁₅ and R₁₆ independently are hydrogen and aliphatic, typically lower aliphatic, such as alkenyl, one example of which is isoprene, as shown below.

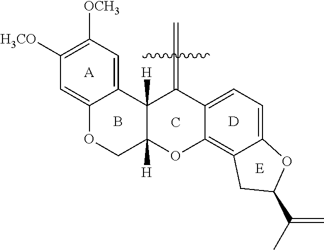

Again, a particular enantiomer is shown in the above formula, but a person of ordinary skill in the art will appreciate that the scope of the present invention is not limited to the particular enantiomer shown. Instead, all stereoisomers that act as haptens also are within the scope of the disclosure. All substitutions discussed above for this class of compounds applies to this particular compound. Other substitutions also are readily apparent to a person of ordinary skill in the art. For example, the methoxy groups on the A ring can be any alkoxy compound, particular lower alkoxy groups. The isoprene unit also provides an olefin that can be synthetically modified, perhaps to provide an alternative position, or at least a second position, for coupling the hapten to a linker or a tyramine or tyramine derivative. For example, the olefin could be converted to an alcohol by hydroboration. It also could be converted to a halide or an epoxide either for use as a hapten or as intermediates useful for further transformation.

A fourth general formula for describing rotenone-based haptens of the present invention is particularly directed to rotenone isoxazolines, as provided below.

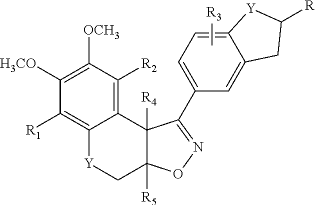

R-R₅ are defined as above, further including all branched chain aliphatic isomers. At least one of the R-R₅ substituents also is bonded to a linker or to a tyramine or tyramine derivative. Y is oxygen, nitrogen, or sulfur.

A particular working embodiment of a rotenone-based hapten satisfying this fourth general formula is provided below.

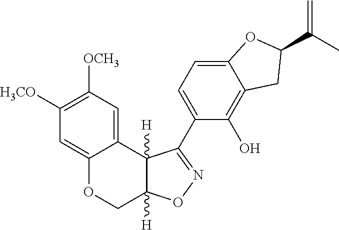

7. Oxazoles and Thiazoles

Oxazole and thiazole sulfonamides provide another class of haptens within the scope of the present invention. A general formula for oxazole and thiazole sulfonamides is provided below.

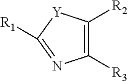

With reference to this first general formula R₁-R₃ are defined as above. Two or more of these R₁-R₃ substituents also may be atoms, typically carbon atoms, in a ring system bonded or fused to the compounds having the illustrated general formula. At least one of the R₁-R₃ substituents is bonded to a linker or is a functional group suitable for coupling to a linker or a tyramine or tyramine derivative. Y is oxygen or sulfur, typically sulfur.

For certain exemplary working embodiments, R₁ has been amido, such as the amide derivatives shown below. R₂ provides a position for coupling to a linker or to a tyramine or tyramine derivative, although the positions indicated by R₁ and R₂ also provide alternative or additional positions for coupling to a linker and/or tyramine or tyramine derivative. R₂, for certain working embodiments, has been —SO₂, and has been used to couple linkers by forming a sulfonamide. Thus, a second general formula for working embodiments of haptens exemplifying this class of haptens is indicated below, where the R₃-R₆ substituents and Y are as stated above.

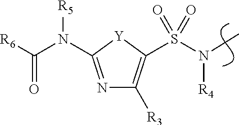

For certain working embodiments R₆ has been alkyl, particularly lower alkyl, such as methyl, and Y has been sulfur.

One working embodiment of a compound according to this class of haptens had the following chemical structure.

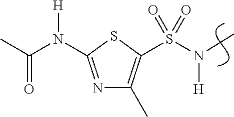

The thiazole or oxazole might also be part of a larger ring system. For example, the 5-membered oxazole or thiazole might be coupled to at least one additional ring, such as a phenyl ring, as indicated below.

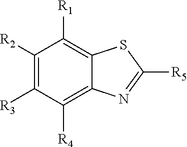

While the R₁-R₅ groups generally can be as stated above, such compounds also provide a position for coupling to a linker and/or to a tyramine or tyramine derivative, such as a R₅. One possible sulfonamide derivative is provided below.

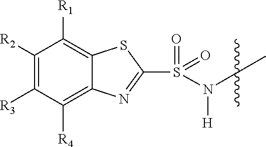

8. Coumarins

Coumarin and coumarin derivatives provide another class of haptens within the scope of the present invention. A general formula for coumarin and coumarin derivatives is provided below.

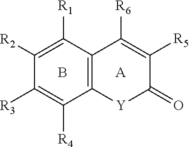

With reference to this general formula, R₁-R₆ are defined as above. At least one of the R₁-R₆ substituents also typically is bonded to a linker or a tyramine or tyramine derivative. Certain working embodiments have used the position indicated as having an R₅ substituent for coupling to a linker or tyramine or tyramine derivative. The 4 position can be important if fluorescence is used to detect these compounds. Substituents other than hydrogen at the 4 position are believed to quench fluorescence, although such derivatives still may be chromophores. Y is oxygen, nitrogen or sulfur. Two or more of the R₁-R₆ substituents available for forming such compounds also may be atoms, typically carbon atoms, in a ring system bonded or fused to the compounds having the illustrated general formula. Exemplary embodiments of these types of compounds are provided below.

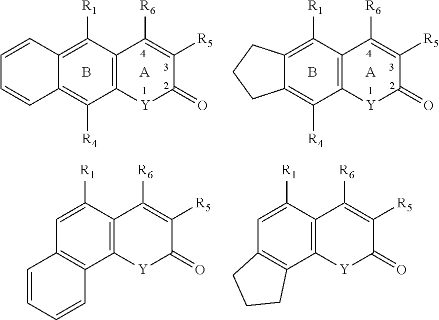

A person of ordinary skill in the art will appreciate that the rings also could be heterocyclic and/or heteroaryl.

Working embodiments typically were fused A-D ring systems having at least one linker, tyramine or tyramine derivative coupling position, with one possible coupling position being indicated below.

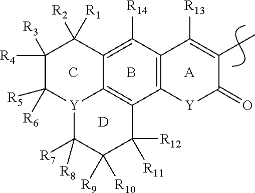

With reference to this general formula, the R and Y variable groups are as stated above. Most typically, R₁-R₁₄ independently are hydrogen or lower alkyl. Particular embodiments of coumarin-based haptens include 2,3,6,7-tetrahydro-11-oxo-1H,5H,11H-[1]benzopyrano[6,7,8-ij]quinolizine-10-carboxylic acid

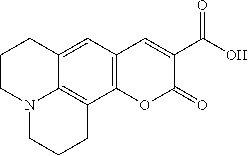

and 7-(diethylamino)coumarin-3-carboxylic acid

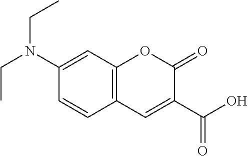 [SMILES: CCN(CC)c1ccc2cc(C(=O)O)c(=O)oc2c1]

9. Cyclolignans

Lignin-based compounds, particularly cyclolignans, such as Podophyllotoxin and derivatives thereof, provide another class of haptens within the scope of the present invention. A first general formula for these cyclolignan-based derivatives is provided below.

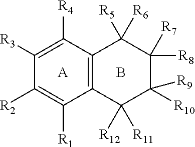

A number of publications discuss naturally occurring, semi-synthetic and synthetic cyclolignans that are useful for describing the genus of cyclolignans useful for practicing the present invention, including: Stephanie Desbene and Sylviane Giorgi-Renault, Drugs that Inhibit Tubulin Polymerization: The Particular Case of Podophyllotoxin and Analogues, Curr. Med. Chem.—Anti-Cancer Agents, 2, 71-90 (2002); M. Gordaliza et al., Podophyllotoxin: Distribution, Sources, Applications and New Cytotoxic Derivatives, Toxicon, 44, 441-459 (2004); Phillipe Meresse et al., Etoposide: Discovery and Medicinal Chemistry, Current Medicinal Chemistry, 11, 2443-2466 (2004); M. Pujol et al., Synthesis and Biological Activity of New Class of Dioxygenated Anticancer Agents, Curr. Med. Chem.—Anti-Cancer Agents, 5, 215-237 (2005); and Youngjae You, Podophyllotoxin Derivatives: Current Synthetic Approaches for New Anticancer Agents, Current Pharmaceutical Design, 11, 1695-1717 (2005); each of which is incorporated herein by reference.

Based on the present disclosure and working embodiments, as well as disclosures provided by these prior publications, and with reference to this first general formula, R₁-R₁₂ are defined as above. At least one of R₁-R₁₂ provides a position for coupling the compound to a linker or to a tyramine or tyramine derivative. Furthermore, certain of the R groups may be atoms in a ring system. For example, R₂ and R₃, as well as two of R₇-R₁₀, can be joined together in a ring system. At least one of R₁₂ and R₁₁ also often is an aryl group, such as a benzene ring or a substituted benzene ring.

Certain working embodiments also satisfied the following second general formula, where the R substituents are as stated above.

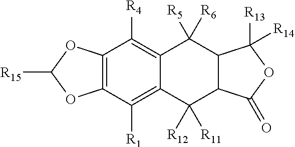

Exemplary compounds where at least one of R₁₁ and R₁₂ is an aryl group have the following general formula, where the R substituents are as stated above.

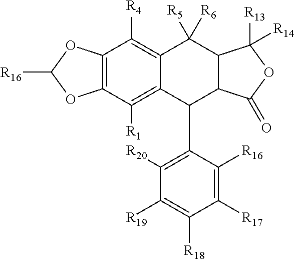

R₁₆-R₂₀ are generally as stated above, but more typically independently are hydrogen or alkoxy, typically lower alkoxy, such as methoxy, as shown below.

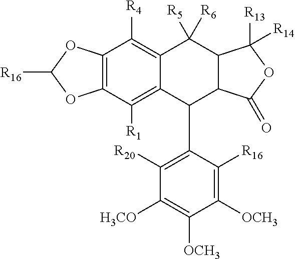

At least one of the R substituents typically is bonded to a linker, is a reactive functional group capable of reacting with a linker, or is -L-RG. For example, R₅ often is -L-RG.

R₅ and R₆ also may form a double bond, such as a double bond to oxygen to form a carbonyl functional group or a double bond to a nitrogen atom to form an imine. Certain exemplary compounds where R₅ and R₆ form a double bond had the following general formula, where the remaining R substituents are as stated above. Y is selected from nitrogen, oxygen or sulfur. If Y is nitrogen, then the nitrogen atom may further have bonded thereto hydrogen, or some atom, functional group or chemical moiety other than hydrogen. For example, the nitrogen may have an aliphatic substituent, such as an alkyl group, an aryl or heteroaryl substituent, or a substituted aryl or heteroaryl substituent, such as an alkyl and/or alkoxy substituted aryl or heteroaryl substituent.

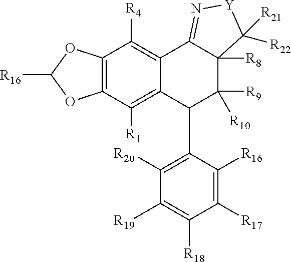

R₁₆-R₂₀ independently are selected from hydrogen and alkoxy, more typically lower alkoxy, such as methoxy, as indicated below.

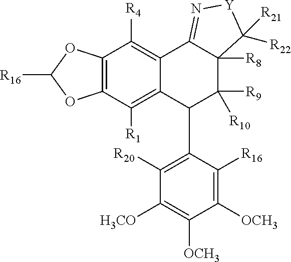

As with all hapten conjugates of the present invention, at least one of the R substituents typically is bonded to a linker, is a reactive functional group capable of reacting with a linker, is -L-RG, or is directly bonded to a tyramine or tyramine derivative. For example, R₉ often is -L-RG.

The chemical structure for Podophyllotoxin, a compound exemplifying this cyclolignan class of haptens, is provided below.

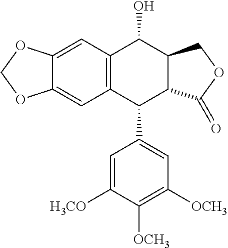 [SMILES: COc1cc([C@@H]2c3cc4c(cc3[C@H](O)[C@H]3COC(=O)[C@H]23)OCO4)cc(OC)c1OC]

Podophyllotoxin, also referred to as podofilox, is a non-alkaloid toxin having a molecular weight of 414.40 and a compositional formula of C₂₂H₂₂^{O}₈.
Podophyllotoxin is present at concentrations of 0.3 to 1.0% by mass in the rhizome of American Mayapple Podophyllum peltatum. The melting point of Podophyllotoxin is 183.3-184.0° C.

Accordingly, cyclolignans according to the present invention based substantially on the Podophyllotoxin structure have the following general formula, where Y is selected from nitrogen, oxygen or sulfur.

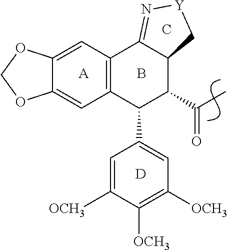

A specific example of a cyclolignan hapten according to the present invention is shown below.

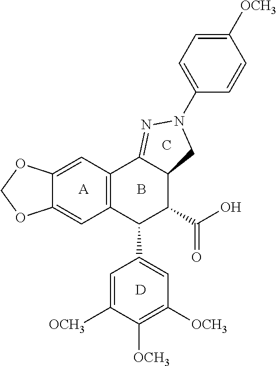

This compound was made starting with Podophyllotoxin. The hydroxyl group of Podophyllotoxin was oxidized to a ketone. The ketone was then reacted with a substituted hydrazine to produce the compound indicated above. The hydrazine reagent can be substituted as desired, including aliphatic and aryl substituents.

10. Heterobiaryl

Another general class of haptens of the present invention is heterobiaryl compounds, typically phenyl quinolines and quinoxalines. Disclosed heterobiaryl compounds have a first general chemical formula as below.

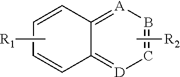

With reference to this general formula, A-D are selected from carbon, nitrogen, oxygen, and sulfur, and any and all combinations thereof. Most typically A-D are carbon or nitrogen, and may be substituted or unsubstituted. R₁-R₂ are defined as above, and further including alkoxy aryl, such as methoxy aryl and ethoxy aryl. Two or more of the R₁-R₂ substituents, most typically plural R₁ substituents, also may be atoms, typically carbon atoms, in a ring system bonded or fused to the compounds having the illustrated general formula. At least one of the R₁-R₂ substituents typically is bonded to a linker or directly to a tyramine or tyramine derivative.

Particular embodiments of the heterobiaryl compounds have the following formula.

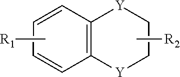

R₁ and R₂ are as stated above for the first general formula. Y is oxygen, nitrogen or sulfur, typically nitrogen. If Y is nitrogen, then the formula also can include double bonds to the one or more nitrogen atoms.

Compounds having a single heteroatom are exemplified by phenylquinolines, such as follows.

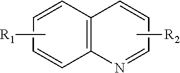

More particular embodiments include aryl substituted haptens, exemplified by the following general formula.

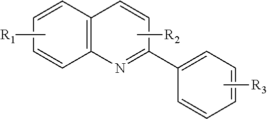

With reference to this general formula, R₁-R₃ are as indicated above. More typically, R₁ is hydrogen, R₂ is acyl, and R₃ is alkoxy. A particular example, 2-(3,4-dimethoxyphenyl)quinoline-4-carboxylic acid, is provided below.

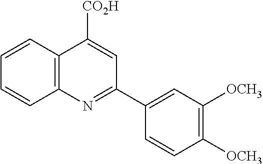

Compounds having two heteroatoms are represented by quinoxalines, as indicated by the general formula below.

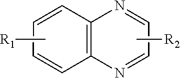

Again, the R₁ and R₂ substituents are as stated above with respect to this class of haptens. A particular example of a biaryl-diheteroatom hapten of the present invention is exemplified by 3-hydroxy-2-quinoxalinecarbamide, below.

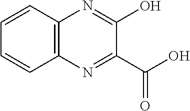 [SMILES: O=C(O)c1nc2ccccc2nc1O]

11. Azoaryl

Another general class of haptens of the present invention is azoaryl compounds, such as azobenzenes, having a first general chemical formula as below.

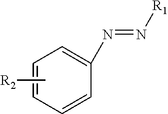

R₁-R₂ are defined as above, and further including alkoxy aryl, such as methoxy aryl and ethoxy aryl. Two or more R₂ substituents also may be atoms, typically carbon atoms, in a ring system bonded or fused to the compounds having the illustrated general formula. For example, 2 R₂ substituents may form a fused phenyl ring, or a fused heterocyclic or heteroaryl structure.

Certain disclosed azoaryl compounds have a first amine substituent and a second aryl substituent. These compounds typically have the following formula.

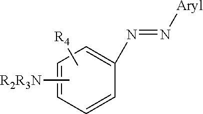

With reference to this general formula, R₂-R₄ are as stated above with respect to this class of haptens, with particular embodiments having R₂-R₃ aliphatic, particularly alkyl, more particularly lower alkyl, and R₄ hydrogen.

A third general formula for describing azoaryl compounds is provided below.

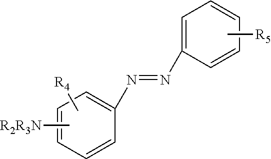

R₂-R₅ are as stated above for this particular class of haptens. At least one of R₂-R₅ defines a position for coupling a linker or tyramine or tyramine derivative to the azoaryl hapten to form a conjugate. For example, R₅ may be a sulfonyl halide functional group. Sulfonyl halides, such as that shown below, are useful functional groups for coupling linkers to the azoaryl haptens.

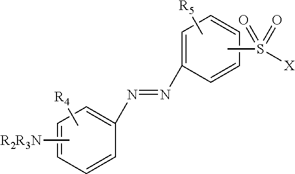

With reference to this formula, R₂-R₅ are as stated above. X is a halide. A particular embodiment of these azoaryl haptens, 4-(dimethylamino)azobenzene-4′-sulfonyl chloride, has the formula provided below.

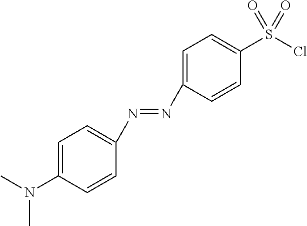 [SMILES: CN(C)c1ccc(/N=N/c2ccc(S(=O)(=O)Cl)cc2)cc1]

12. Benzodiazepines Another class of haptens according to the present invention is the benzodiazepine haptens, having a first general formula as indicated below.

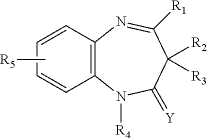

R₁-R₅ are defined as above. Two or more of the R₅ substituents also may be atoms, typically carbon atoms, in a ring system bonded or fused to the compounds having the illustrated general formula. At least one of the R₁-R₅ positions is bonded to a linker or is occupied by a functional group suitable for coupling to a linker or a tyramine or tyramine derivative. R₁-R₅ most typically are aliphatic, aryl, hydrogen, or hydroxyl, even more typically alkyl, hydrogen or phenyl. Y is oxygen or sulfur, most typically oxygen.

Particular embodiments of the benzodiazepine haptens have R₁ aryl, as indicated below.

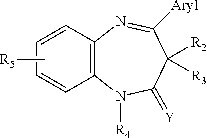

For these embodiments, R₂-R₅ are as stated above for this class of haptens, more typically such substituents are independently selected from aliphatic, particular alkyl, hydrogen and hydroxyl. Certain disclosed embodiments are phenyl compounds, as illustrated below.

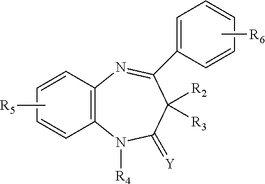

Again, R₂-R₆ are as stated above, but more typically such substituents are independently selected from aliphatic, particularly alkyl, hydrogen and hydroxyl. Certain disclosed embodiments are phenyl compounds, as illustrated below. A particular embodiment, 4-(2-hydroxyphenyl)-1H-benzo[b][1,4]diazepine-2(3H)-one, is provided below.

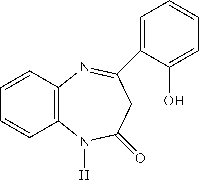 [SMILES: O=C1CC(c2ccccc2O)=Nc2ccccc2N1]

### III. Linkers

1. General

As indicated by the general formula

hapten-optional linker-tyramine/tyramine derivative

conjugates of the present application may include linkers. Any linker currently known for this purpose, or developed in the future, can be used to form conjugates of the present invention by coupling to the haptens disclosed herein. Useful linkers can either be homo- or heterobifunctional, but more typically are heterobifunctional.

2. Aliphatic

Solely by way of example, and without limitation, a first class of linkers suitable for forming disclosed hapten conjugates are aliphatic compounds, such as aliphatic hydrocarbon chains having one or more sites of unsaturation, or alkyl chains. The aliphatic chain also typically includes terminal functional groups, including by way of example and without limitation, a carbonyl-reactive group, an amine-reactive group, a thiol-reactive group or a photo-reactive group, that facilitate coupling to haptens and other desired compounds, such as tyramine. The length of the chain can vary, but typically has an upper practical limit of about 30 atoms. Chain links greater than about 30 carbon atoms have proved to be less effective than compounds having smaller chain links. Thus, aliphatic chain linkers typically have a chain length of from about 1 carbon atom to about 30 carbon atoms. However, a person of ordinary skill in the art will appreciate that, if a particular linker has greater than 30 atoms, and still operates efficiently for linking the hapten to a tyramine or tyramine derivative, and the conjugate still functions as desired, then such chain links are within the scope of the present invention.

3. Alkylene Oxides

A second class of linkers useful for practicing embodiments of the present disclosure are the alkylene oxides. The alkylene oxides are represented herein by reference to glycols, such as ethylene glycols. Hapten conjugates of the present invention have proved particularly useful if the hydrophilicity of the linker is increased relative to their hydrocarbon chains. As a result, the alkylene oxides, such as the glycols, have proved useful for practicing this invention. A person of ordinary skill in the art will appreciate that, as the number of oxygen atoms increases, the hydrophilicity of the compound also may increase. Thus, linkers of the present invention typically have a formula of (—OCH₂CH₂O—)ₙ where n is from about 2 to about 15, but more particularly is from about 2 to about 8.

Heterobifunctional polyalkyleneglycol linkers useful for practicing certain disclosed embodiments of the present invention are described in assignee's co-pending applications, including “Nanoparticle Conjugates,” U.S. patent application Ser. No. 11/413,778, filed Apr. 28, 2006; “Antibody Conjugates,” U.S. application Ser. No. 11/413,418, filed Apr. 27, 2006; and “Molecular Conjugate,” U.S. application Ser. No. 11/603,425, filed Nov. 21, 2006; all of which applications are incorporated herein by reference. A person of ordinary skill in the art will appreciate that the linkers disclosed in these applications can be used to link specific binding moieties, signal generating moieties and haptens in any and all desired combinations. Heterobifunctional polyalkyleneglycol linkers are disclosed below, and their use exemplified by reference to coupling tyramine to haptens and detectable labels.

One particular embodiment of a linker for use with disclosed conjugates is a heterobifunctional polyalkyleneglycol linker having the general structure shown below:

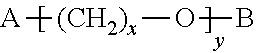

wherein A and B include different reactive groups, x is an integer from 2 to 10 (such as 2, 3 or 4), and y is an integer from 1 to 50, for example, from 2 to 30 such as from 3 to 20 or from 4 to 12. One or more hydrogen atoms can be substituted for additional functional groups such as hydroxyl groups, alkoxy groups (such as methoxy and ethoxy), halogen atoms (F, Cl, Br, I), sulfato groups and amino groups (including mono- and di-substituted amino groups such as dialkyl amino groups.

A and B of the linker can independently include a carbonyl-reactive group, an amine-reactive group, a thiol-reactive group or a photo-reactive group, but are not the same. Examples of carbonyl-reactive groups include aldehyde- and ketone-reactive groups like hydrazine derivatives and amines. Examples of amine-reactive groups include active esters such as NHS or sulfo-NHS, isothiocyanates, isocyanates, acyl azides, sulfonyl chlorides, aldehydes, glyoxals, epoxides, oxiranes, carbonates, aryl halides, imidoesters, anhydrides and the like. Examples of thiol-reactive groups include non-polymerizable Michael acceptors, haloacetyl groups (such as iodoacetyl), alkyl halides, maleimides, aziridines, acryloyl groups, vinyl sulfones, benzoquinones, aromatic groups that can undergo nucleophilic substitution such as fluorobenzene groups (such as tetra and pentafluorobenzene groups), and disulfide groups such as pyridyl disulfide groups and thiols activated with Ellman's reagent. Examples of photo-reactive groups include aryl azide and halogenated aryl azides. Alternatively, A and/or B can be a functional group that reacts with a specific type of reactive group. For example, A and/or B can be an amine group, a thiol group, or a carbonyl-containing group that will react with a corresponding reactive group (such as an amine-reactive group, thiol-reactive group or carbonyl-reactive group, respectively) that has been introduced or is otherwise present on a hapten and/or a tyramine or tyramine derivative. Additional examples of each of these types of groups will be apparent to those skilled in the art. Further examples and information regarding reaction conditions and methods for exchanging one type of reactive group for another are provided in Hermanson, “Bioconjugate Techniques,” Academic Press, San Diego, 1996, which is incorporated by reference herein. In a particular embodiment, a thiol-reactive group is other than vinyl sulfone.

In some embodiments the heterobifunctional linker has the formula:

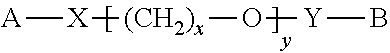

wherein A and B are different reactive groups and are as stated above; x and y are as stated above, and X and Y are additional spacer groups, for example, spacer groups having between 1 and 10 carbons such as between 1 and 6 carbons or between 1 and 4 carbons, and optionally containing one or more amide linkages, ether linkages, ester linkages and the like. Spacers X and Y can be the same or different, and can be straight-chained, branched or cyclic (for example, aliphatic or aromatic cyclic structures), and can be unsubstituted or substituted. Functional groups that can be substituents on a spacer include carbonyl groups, hydroxyl groups, halogen (F, Cl, Br and I) atoms, alkoxy groups (such as methoxy and ethoxy), nitro groups, and sulfate groups.

In particular embodiments, the heterobifunctional linker comprises a heterobifunctional polyethylene glycol linker having the formula:

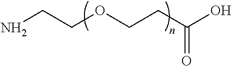

wherein n=1 to 50, for example, n=2 to 30 such as n=3 to 20 or n=4 to 12. In particular embodiments, n=4 or 8.

### IV. Hapten Conjugates

Hapten conjugates include a hapten, a peroxidase-activatable aryl moiety, and optionally a linker. In certain embodiments, the hapten and linker are conjugated to the peroxidase activatable moiety and have the general formula

hapten-optional linker-peroxidase-activatable aryl moiety

In some embodiments, the peroxidase activatable aryl moiety is tyramine or a tyramine derivative. In certain embodiments, the hapten and optional linker are conjugated to tyramine and have the general formula

hapten-optional linker-tyramine

In other embodiments, the hapten and optional linker are conjugated to a tyramine derivative and have the following general formula.

hapten-optional linker-tyramine derivative

Embodiments of tyramine derivatives have the general formula

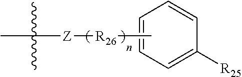

where R₂₅ is selected from hydroxyl, ether, amine, and substituted amine; R₂₆ is selected from alkyl, alkenyl, alkynyl, aryl, heteroaryl, —ORₘ, —NRₘ, and —SRₘ, where m is 1-20; n is 1-20; Z is selected from oxygen, sulfur, or NRₐ where Rₐ is selected from hydrogen, aliphatic, aryl, or alkyl aryl. Thus, the conjugate has the following general formula.

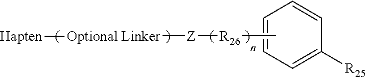

In some embodiments, the hapten is selected from oxazoles, pyrazoles, thiazoles, nitroaryls, benzofurazans, triterpenes, ureas, thioureas, rotenones, coumarins, podophyllotoxin-based compounds, and combinations thereof. The linker, if present, may be aliphatic, heteroaliphatic, or heterobifunctional.

In certain embodiments, the conjugate has a general formula as shown in Table 1 below. In each of the general formulas in Table 1, the substituents for each R group, X, Y, and Z, are as recited above in discussions of haptens, linkers, and tyramide derivatives.

TABLE 1

| Hapten Class | General Formula |
|---|---|
| Azole | 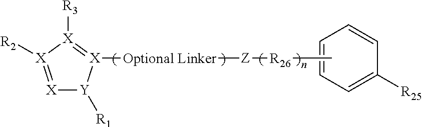 |
| Nitroaryl | 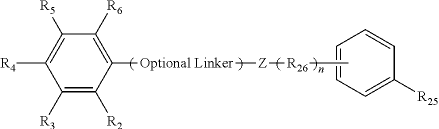 |
| Benzofurazan | 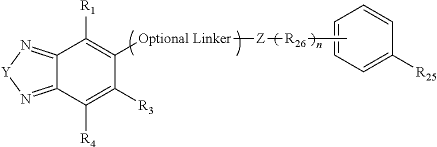 |
| Urea/thiourea | 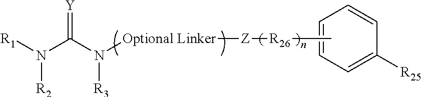 |
| Triterpene | 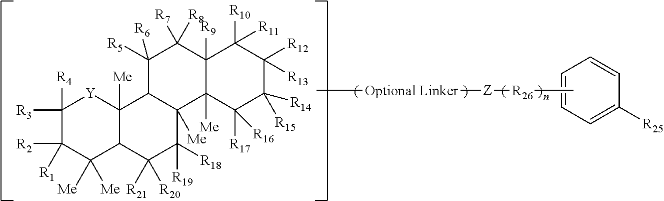 |
| Rotenone | 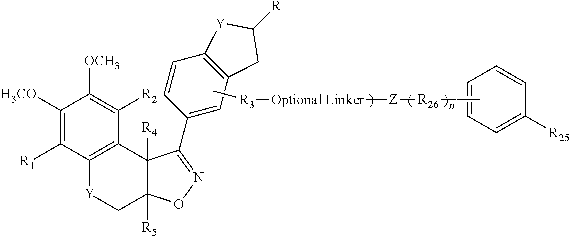 |
| Oxazole/Thiazole sulfonamide | 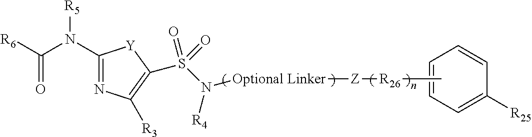 |
| Cyclolignan | 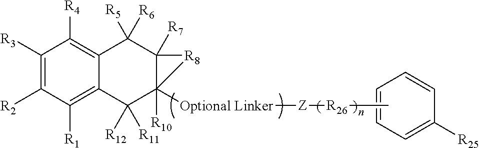 |
| Heterobiaryl | 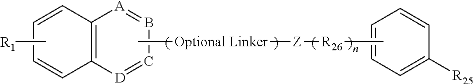 |
| Azoaryl | 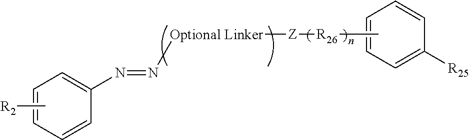 |
| Benzodiazepine | 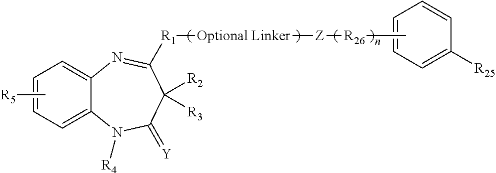 |
| Coumarin | 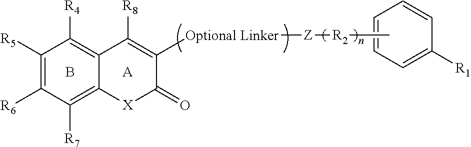 |

In particular embodiments, the conjugate is a hapten-tyramide conjugate with a formula as shown in Table 2.

TABLE 2

| | |
|---|---|
| 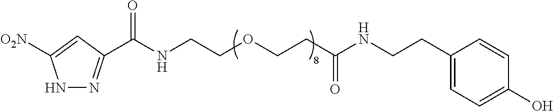 | 1 |
| 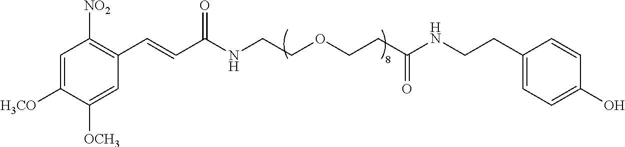 | 2 |
| 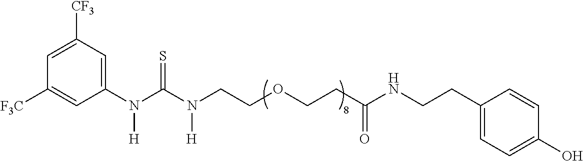 | 3 |
| 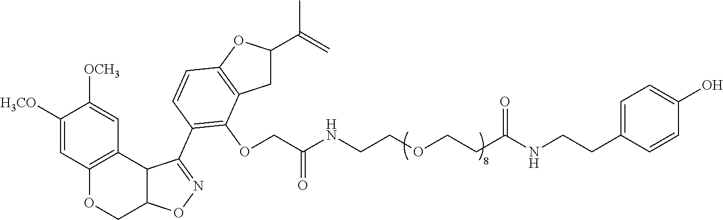 | 4 |
| 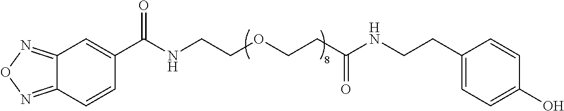 | 5 |
| 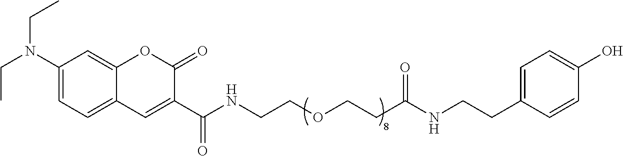 | 6 |
| 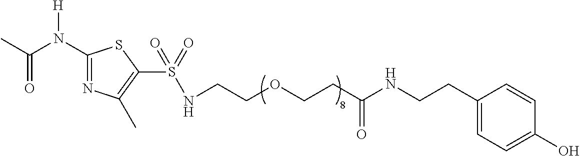 | 7 |
| 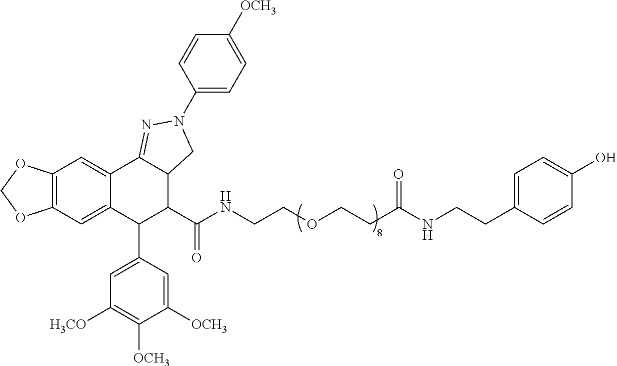 | 8 |
| 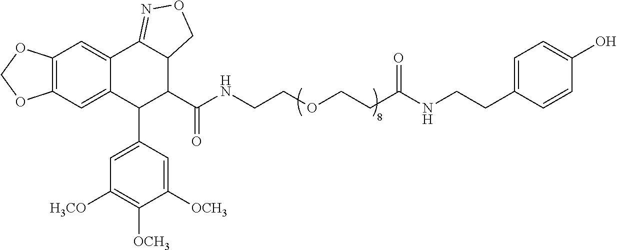 | 9 |
| 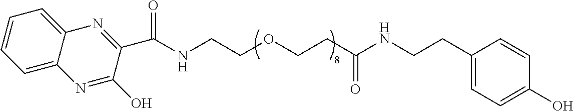 | 10 |
| 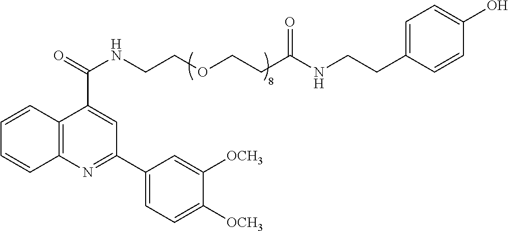 | 11 |
| 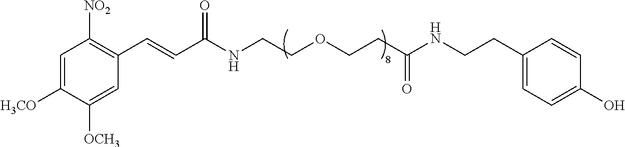 | 12 |
| 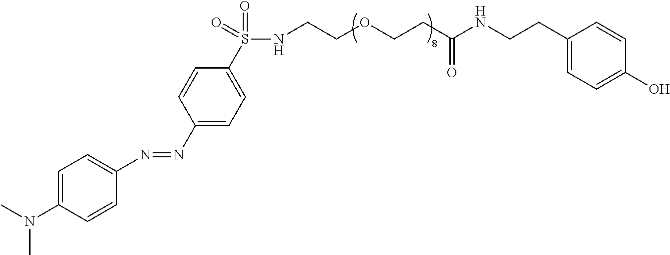 | 13 |
| 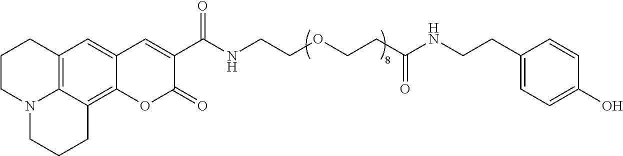 | 14 |
| 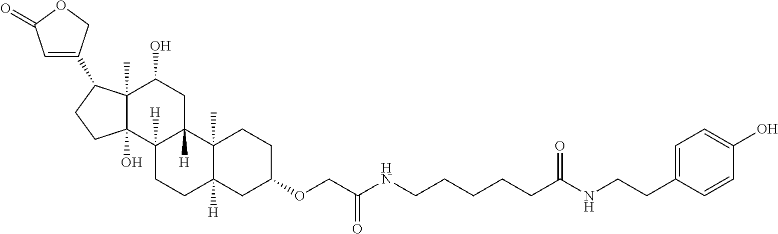 | 15 |

### V. Methods for Making Hapten Conjugates

In some embodiments, a hapten having a electrophilic functional group, or having a functional group capable of being converted to an electrophilic functional group, is conjugated to a compound comprising a peroxidase-activatable aryl moiety or to a linker, e.g., an aliphatic or poly(alkylene oxide) linker. In certain embodiments, the hapten includes a carboxylic acid functional group, which is converted to an activated, electrophilic carbonyl-containing functional group, such as, but not limited to, an acyl halide, an ester (e.g., a N-hydroxysuccinimide ester), or an anhydride. The peroxidase-activatable aryl moiety includes a nucleophilic functional group (e.g., amino, hydroxyl, thiol, or anions formed therefrom) capable of reacting with the hapten's activated electrophilic functional group. The hapten's electrophilic group can be coupled to the peroxidase-activatable aryl moiety's nucleophilic group using organic coupling techniques known to a person of ordinary skill in the art of organic chemistry synthesis. In embodiments where the conjugate includes a linker, the linker typically has a nucleophilic functional group at one end and an electrophilic functional group at the other end. The linker's nucleophilic group can be coupled to the hapten's electrophilic group, and the linker's electrophilic group can be activated and coupled to the peroxidase-activatable aryl moiety's nucleophilic group using organic coupling techniques known to a person of ordinary skill in the art of organic chemistry synthesis.

In one embodiment, as shown in Scheme 1 below, a hapten having a carboxylic acid functional group is conjugated to tyramine via a linker. The hapten is coupled to N-hydroxysuccinimide (NHS) to produce a hapten-NHS ester. The reaction is performed in a solvent in which the hapten and NHS are soluble; one suitable solvent is dichloromethane. In some embodiments, N,N′-dicyclohexyl-carbodiimide is utilized as the coupling agent. The urea byproduct is removed by filtration, and the active ester can be used without further purification.

In some embodiments, the hapten-NHS ester is coupled to a linker. For example, the hapten-NHS ester may be coupled to a polyethylene glycol (PEG) linker using a PEG amino acid, and is converted to the corresponding amide by reaction with the PEG amino acid under basic conditions (e.g., in a solution of triethylamine and dichloromethane). In working embodiments, a dPEG®_{g} amino acid (Quanta BioDesign Ltd., Powell, Ohio) was used. The product can be purified via flash chromatography.

The hapten-containing linker is activated by reaction with NHS and N,N′-dicyclohexyl-carbodiimide at room temperature to produce the corresponding NHS ester of the carboxy-PEG-hapten. The urea byproduct is removed by filtration, and the NHS ester can be used without further purification.

The desired hapten-tyramide conjugate is obtained by displacement of the succinimide moiety of the NHS ester with tyramine. The reaction is performed in a solvent in which the NHS ester is soluble; one suitable solvent is N,N′-dimethylformamide (DMF). The product can be purified via flash chromatography.

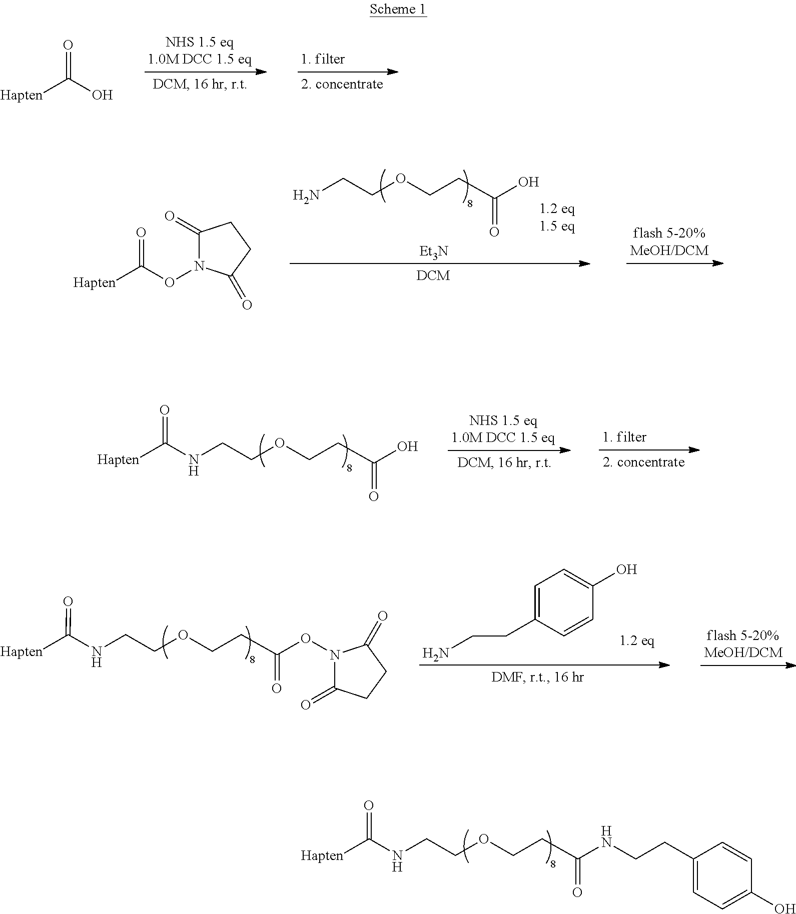

In one embodiment, the hapten is 3-hydroxyquinoxaline-2-carboxylic acid, and the coupling agent is 1-ethyl-3-(3-dimethylaminopropyl)carbodiimide (EDAC). The hapten and NHS ester are dissolved in a suitable solvent, e.g., DMF. The hapten-NHS ester is insoluble in DMF, and can be collected via filtration. The remainder of the reaction is performed as outlined in Scheme 1, with the linker coupling being performed in DMF/triethylamine.

### V. Methods of Using Hapten Conjugates

Embodiments of the disclosed hapten conjugates can be utilized in signal amplification assays. Signal amplification utilizes the catalytic activity of a peroxidase enzyme to covalently bind a peroxidase-activatable aryl moiety to a solid phase. The solid phase may be, for example, protein components of cells or cellular structures that are immobilized on a substrate such as a microscope slide. Some peroxidase enzymes (e.g., horseradish peroxidase), in the presence of a peroxide, catalyze the dimerization of certain compounds, e.g., phenolic compounds, probably by the generation of free radicals. Thus, if a peroxidase-activatable aryl moiety is added to a protein-containing sample in the presence of horseradish peroxidase and peroxide (e.g., hydrogen peroxide), the peroxidase-activatable aryl moiety can form a free radical and subsequently form a dimer with the phenol group of a tyrosine amino acid. It is desirable, however, to specifically bind the peroxidase-activatable aryl moiety at, or in close proximity to, a desired target with the sample. This objective can be achieved by immobilizing the enzyme on the target region, as described below. Only peroxidase-activatable aryl moieties in close proximity to the immobilized enzyme will react and form dimers with tyrosine residues in the vicinity of, or proximal to, the immobilized enzyme, including tyrosine residues in the enzyme itself, tyrosine residues in the antibody to which the enzyme is conjugated, and/or tyrosine residues in the sample that are proximal the immobilized enzyme, such as within about 100 nm, within about 50 nm, within about 10 nm, or within about 5 nm of the immobilized enzyme. For example, the tyrosine residue may be within a distance of about 10 angstroms to about 100 nm, about 10 angstroms to about 50 nm, about 10 angstroms to about 10 nm, or about 10 angstroms to about 5 nm from the immobilized enzyme. Such proximal binding allows the target to be detected with at least the same degree of specificity as conventional staining methods used with IHC and/or ISH. For example, embodiments of the disclosed method allow sub cellular structures to be distinguished, e.g., nuclear membrane versus the nuclear region, cellular membrane versus the cytoplasmic region, etc.

In some embodiments, the hapten conjugate is a hapten-tyramide conjugate that can be utilized in a tyramide signal amplification assay. Tyramide signal amplification is a peroxidase-based signal amplification system that is compatible with in situ hybridization (ISH), immunocytochemical, and immunohistochemical (IHC) detection schemes. Tyramide signal amplification assays may be “direct” or “indirect.” A direct tyramide signal amplification assay is performed when a label, e.g., a fluorescent label, is bound to the tyramine to form a label-tyramide conjugate, and the label is detected directly after the label-tyramide conjugate is bound to the sample. An indirect tyramide signal amplification assay is performed when a hapten is bound to the tyramine. A fluorescent or enzyme-labeled anti-hapten antibody is used to detect the hapten.

In disclosed embodiments, a signal amplification assay typically includes the following steps: a) immobilizing an enzyme on a target in a sample; b) contacting the sample with a hapten conjugate in such a manner that the enzyme is capable of reacting with the hapten conjugate, thereby causing the hapten conjugate to bind to the sample proximal to the immobilized enzyme; c) contacting the sample with a labeled anti-hapten antibody that is capable of binding to the hapten; and d) locating, or visualizing, the target in the sample by detecting the labeled anti-hapten antibody by any suitable means. In certain embodiments, the hapten conjugate is a hapten-tyramide conjugate. The target can be any molecule of interest for which the presence, location and/or concentration is to be determined. Examples of molecules of interest include proteins and nucleic acid sequences.

Typically the sample contains proteins, such as a tissue sample. Typically, the immobilized enzyme is a peroxidase enzyme capable of reacting with a peroxidase-activatable aryl moiety, e.g., tyramide. In some embodiments, the enzyme is immobilized on the target by incubating the sample with an enzyme conjugate that binds to the target. The enzyme may be conjugated to any moiety capable of binding to the target. Suitable moieties include, but are not limited to, antibodies, nucleotides, oligonucleotides, proteins, peptides, or amino acids.

In other embodiments, immobilizing the enzyme is a multi-step process. For example, the sample may be incubated with a first moiety (e.g., an antibody, nucleotide, oligonucleotide, protein, oligopeptide, peptide, or amino acid) that binds to the target. The sample then may be incubated with an enzyme conjugate comprising a moiety that is capable of binding to the first moiety. In some embodiments where the first moiety is an antibody to the target, the two-step process may be more versatile because it allows the user to employ a “universal” enzyme-antibody conjugate. For example, if the first antibody is a rabbit monoclonal antibody, the enzyme-antibody conjugate may include an antibody that is capable of binding to any rabbit monoclonal antibody. The multi-step process can eliminate the need to generate an enzyme-antibody conjugate that is suitable for each target.

In some embodiments, the first moiety may be a labeled probe, such as a labeled oligonucleotide. After the probe has been hybridized to the sample, a first antibody that recognizes the label is introduced and binds to the labeled probe. The first antibody may be an enzyme-antibody conjugate. However, if the first antibody is not conjugated to an enzyme, an enzyme-antibody conjugate is introduced wherein the antibody moiety of the conjugate recognizes and binds to the first antibody.

Once the enzyme is immobilized on the sample, the hapten conjugate is introduced under suitable conditions to enable the enzyme to react with the peroxidase-activatable aryl moiety. Typically the enzyme is a peroxidase, such as horseradish peroxidase. Thus, suitable conditions include a reaction buffer, or solution, that includes a peroxide (e.g., hydrogen peroxide), and has a salt concentration and pH that enable the enzyme to perform its desired function. The reaction is performed at a temperature that is suitable for the enzyme. For example, if the enzyme is horseradish peroxidase, the reaction may be performed at 35-40° C. Under such conditions, the peroxidase-activatable aryl moiety reacts with the peroxide and the enzyme, converting the peroxidase-activatable aryl moiety to an active form that covalently binds to the sample, typically by binding to a tyrosine residue proximal to the immobilized enzyme, including tyrosine residues within the immobilized enzyme itself.

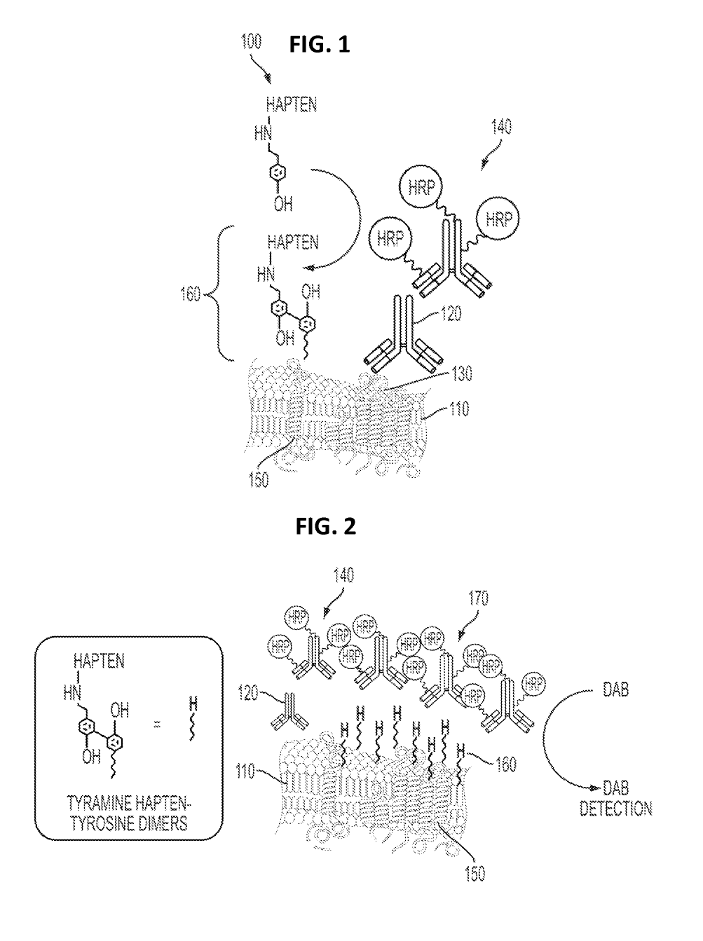

FIG. 1 is a schematic diagram illustrating one embodiment of a method for binding a hapten conjugate, such as a hapten-tyramide conjugate 100, to an immobilized tissue sample 110. A primary antibody 120 binds to an epitope 130 within an immobilized tissue sample 110. A secondary antibody 140 is introduced and binds to the primary antibody 120. If, for example, the primary antibody is a mouse IgG antibody, the secondary antibody may be an anti-mouse antibody that will bind to any mouse IgG antibody. In FIG. 1, a horseradish peroxidase-antibody conjugate 140 includes the secondary antibody. The hapten-tyramide conjugate 100 is added. In the presence of horseradish peroxidase (HRP) and peroxide (e.g., hydrogen peroxide), the hapten-tyramide conjugate 100 becomes covalently bound proximal to the enzyme site. The conjugate can bind to a tyrosine residue within horseradish peroxidase antibody conjugate 140, a tyrosine residue within primary antibody 120, or a tyrosine residue, e.g., in a protein 150, within sample 110. FIG. 1 illustrates a dimer 160 formed when the phenol group of tyramine binds to the phenol group of a tyrosine residue in the protein.

After the hapten conjugate is bound to the sample, its presence is detected by suitable means. In some embodiments, the hapten may be detected directly. For example, a hapten conjugated to a quantum dot may be detected via the quantum dot's fluorescence at a characteristic wavelength. In other embodiments, the hapten is detected indirectly. For example, an anti-hapten antibody may be introduced and bound to the hapten. In certain embodiments, the anti-hapten antibody is a conjugate comprising the antibody and a detectable label. In other embodiments, a label-antibody conjugate that recognizes the anti-hapten antibody subsequently is introduced and bound to the anti-hapten antibody. The label is detected by suitable means.

FIG. 2 illustrates one embodiment of a method for detecting hapten-tyramide/tyrosine dimers 160. An anti-hapten antibody 170 is introduced. The anti-hapten antibody 170 typically is a conjugate comprising the antibody and a label (e.g., a fluorophore or other directly-detectable label) or an enzyme (e.g., horseradish peroxidase (HRP), alkaline phosphatase, etc.) In the illustrated embodiment, the anti-hapten antibody 170 is an HRP-antibody conjugate. The anti-hapten antibody 170 binds to the hapten portion of the hapten-tyramide/tyrosine dimer 160. The anti-hapten antibody 170 then is detected by any suitable method. For example, when the anti-hapten antibody is an HRP-antibody conjugate, a 3,3′-diaminobenzidine (DAB) assay may be used for chromogenic detection of the HRP. In other embodiments, the anti-hapten antibody may be a fluorophore-antibody conjugate, and the fluorophore (e.g., a quantum dot) may be detected by its fluorescence.

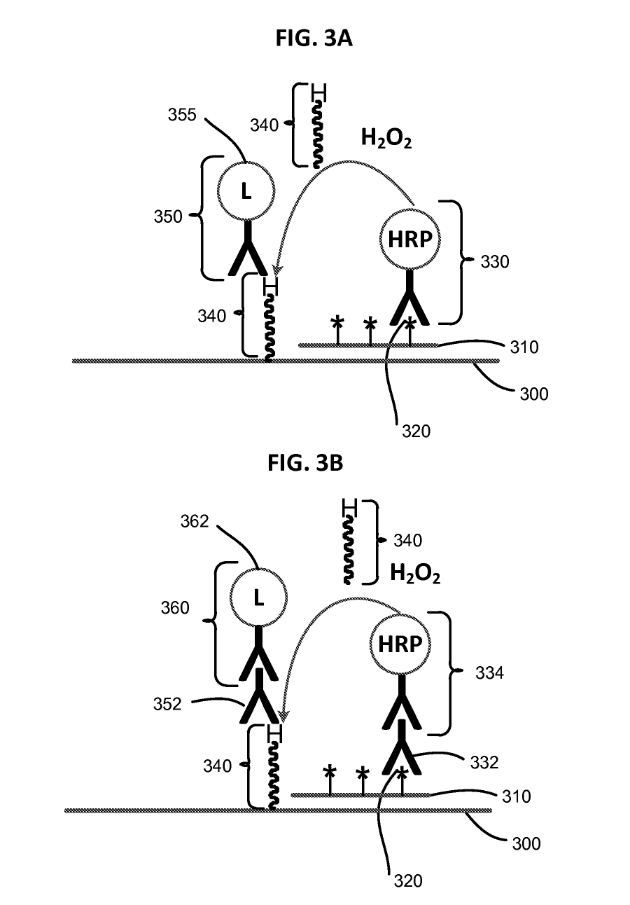

FIG. 3A illustrates one embodiment of a method for detecting a target oligonucleotide sequence in a sample using a hapten conjugate. A sample 300 including a target oligonucleotide sequence is provided. A complementary probe 310 that includes a label 320 (e.g., a labeled DNA, RNA, or oligonucleotide probe) is introduced and binds to the target sequence in the sample 300. An anti-label antibody-enzyme conjugate 330 (e.g., an anti-label antibody conjugated to HRP) is added and binds to the label 320. A hapten conjugate, e.g., a hapten-tyramide conjugate 340, is introduced. In the presence of HRP and peroxide, the hapten tyramide conjugate 340 reacts with a tyrosine residue (e.g., a tyrosine residue in antibody-enzyme conjugate 330 or within sample 300), and becomes covalently bound proximal to antibody-enzyme conjugate 330. An anti-hapten antibody-label conjugate 350 is added and binds to the hapten-tyramide conjugate 340. The label 355 is detected by suitable means. In a working embodiment, label 355 was a Qd655 quantum dot, and its fluorescence at 655 nm was detected using a fluorescent microscope.

FIG. 3B illustrates another embodiment of a method for detecting a target oligonucleotide sequence in a sample using a hapten conjugate. A sample 300 including a target oligonucleotide sequence is provided. A complementary probe 310 that includes a label 320 (e.g., a labeled DNA, RNA, or oligonucleotide probe) is introduced and binds to the target sequence in the sample 300. In a working embodiment, the label 320 was DNP. An anti-label antibody 332 (e.g., an anti-DNP antibody) is added and binds to the label 320. An enzyme-antibody conjugate 334 (e.g., an antibody conjugated to HRP) subsequently binds to antibody 332. A hapten-tyramide conjugate 340 is introduced. In the presence of HRP and peroxide, the hapten-tyramide conjugate 340 reacts with a tyrosine residue (e.g., a tyrosine residue in enzyme-antibody conjugate 334, in antibody 332, or within sample 300), and becomes covalently bound proximal to enzyme-antibody conjugate 334. An anti-hapten antibody 352 is added and binds to the hapten-tyramide conjugate 340. Next, a labeled antibody 360 that recognizes and binds to the anti-hapten antibody 352 is added. The label 362 is detected by suitable means. In a working embodiment, label 362 was a Qd655 quantum dot, and its fluorescence at 655 nm was detected using a fluorescent microscope.

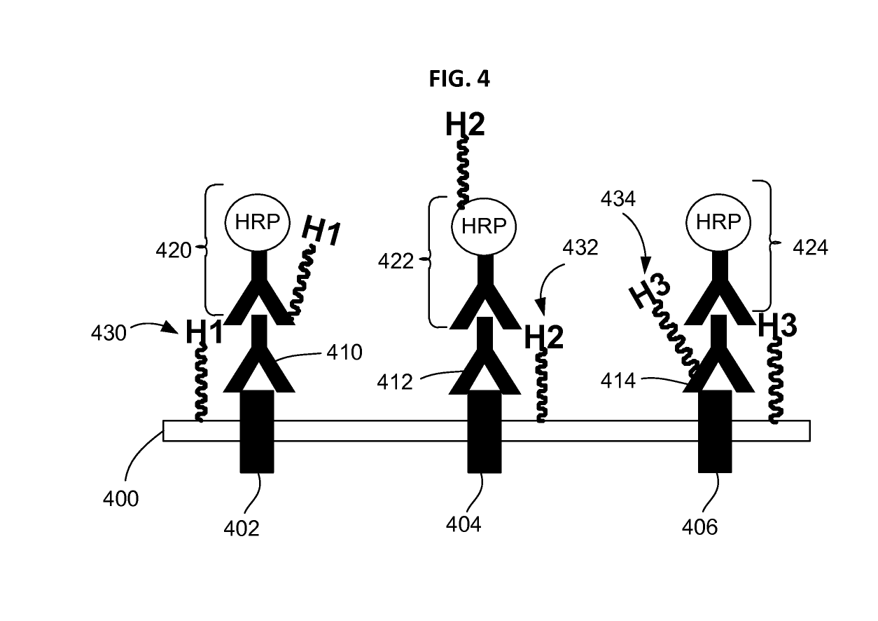

In some embodiments, hapten conjugates are used for multiplexed detection of different protein and/or oligopeptide targets in a sample. Multiplexing can be performed with immunohistochemistry (IHC), in situ hybridization (ISH), fluorescent IHC/ISH, or any combination thereof. FIG. 4 illustrates one embodiment of a method for multiplexed detection of three protein and/or oligopeptide targets. Sample 400 includes a plurality of targets 402, 404, 406. A first primary antibody 410 binds to first target 402. A first antibody-peroxidase conjugate 420 is introduced and binds to the primary antibody 410. A first hapten conjugate 430 is added. In the presence of peroxide and the peroxidase, the hapten conjugate 430 becomes covalently bound proximal to first target 402. In some embodiments, first antibody-peroxidase conjugate 420 is deactivated, such as by addition of an excess of peroxide, and the sample is washed to remove excess peroxide. Deactivation can be performed to eliminate any reaction between the first peroxidase and a subsequent hapten-tyramide conjugate. A second primary antibody 412 then is added and binds to second target 404. A second antibody-peroxidase conjugate 422 is introduced and binds to second primary antibody 412. A second hapten conjugate 432 is added. In the presence of peroxide and the peroxidase, second hapten conjugate 432 becomes covalently bound proximal to second target 404. In some embodiments, second antibody-peroxidase conjugate 422 is deactivated, such as by addition of an excess of peroxide, and the sample is washed to remove excess peroxide. A third primary antibody 414 then is added and binds to third target 406. A third antibody-peroxidase conjugate 424 is introduced and binds to third primary antibody 414. A third hapten conjugate 434 is added. In the presence of peroxide and the peroxidase, third hapten conjugate 434 becomes covalently bound proximal to third target 406.

In particular embodiments, antibody-peroxidase conjugates 420, 422, 424 are the same and include an antibody capable of recognizing all three primary antibodies. For example, if primary antibodies 410, 412, 414 are mouse monoclonal antibodies specific for their respective targets, then the antibody-peroxidase conjugates may include a goat anti-mouse antibody.

Typically hapten conjugates 430, 432, 434, include haptens that are different from one another. The haptens are detected using embodiments of the methods described above. Typically a different label is used to detect each of the haptens so that the three targets 402, 404, 406 can be distinguished from one another.

It was unexpectedly discovered that the utility of at least some haptens in a tyramide signal amplification assay with a hapten-tyramide conjugate is unpredictable compared to the hapten's utility in a direct binding assay. In fact, the utility of certain haptens in a tyramide signal amplification assay was inversely correlated to the hapten's utility in a direct binding assay. For example, e.g. some haptens (e.g., DIG and DNP) produce a robust signal when used in a direct assay, such as when a hapten-antibody complex binds to a target. However, some robust haptens were unacceptable for use in a tyramide signal amplification assay where they produced high background noise, resulting in a low signal:background noise ratio. For instance, when used in a screening assay to visualize an antibody on tonsil tissue (see Example 2), a DIG-tyramide conjugate produced a signal/noise ratio of 1.33 when it was applied at a concentration of 5.5 μM. At a concentration of 55 μM, the signal:noise ratio was 1.07. A DNP-tyramide conjugate produced a signal:noise ratio of 2.67 at 5.5 μM, and a ratio of 2 at 55 μM. Conversely, other haptens, which provide only weak detection in a direct assay, produced surprisingly superior results. For example, HQ-, rhodamine-, and DABSYL-tyramide conjugates each produced a signal:noise ratio of 16 when applied at a concentration of 55 μM. A rotenone conjugate produced a signal:noise ratio of 15.

Unexpected results also were found in an mRNA-ISH assay comparing the signals obtained when haptens were directly bound to a probe and the signals obtained when tyramide signal amplification was performed using hapten-tyramide conjugates (see Example 3). The results showed that the performance of a particular hapten-tyramide conjugate could not be predicted from the performance of a corresponding haptentylated RNA probe. Surprisingly, BD-, DIG-, HQ-, and NCA-tyramide conjugates all produced strong signals, while their respective haptenylated probes produced little or no signal.

In some embodiments, hapten conjugates are used for multiplexed detection of multiple genes in a tissue sample using an RNA-ISH assay (see Example 5). The multiplexed assay allows simultaneous visualization and evaluation of gene expression from multiple target genes. Gene expression data can influence therapy selection for cancer patients. For example, mRNA levels corresponding to particular genes implicated in breast cancer are correlated to patient risk. Exemplary mRNA targets related to breast cancer risk and assessment include proliferation targets (e.g., Ki-67, STK15, Survivin, Cyclin B1, MYBL2), invasion targets (e.g., Stromelysin 3, Cathepsin L2), HER2 targets (e.g., HER2, GRB7), estrogen targets (e.g., ER, PGR, Bcl2, SCUBE2), and other targets (e.g., GSTM1, CD68, BAG1). Determining the RNA levels can provide a clinician with data useful for developing a specific treatment plan for each patient.

A tissue sample is obtained and fixed. Hapten-labeled probes capable of hybridizing to particular RNA targets of interest are prepared and hybridized with the fixed tissue sample. Each probe is labeled with a different hapten, and hybridizes to a different RNA target. In some embodiments, hybridization signals are increased using signal amplification to increase the number of haptens deposited in each probe's vicinity. The haptens are detected using anti-hapten antibodies conjugated to quantum dots capable of fluorescing at distinct wavelengths from one another. The multiplexed RNA-ISH assay produces punctate signals for each target in the sample, allowing simultaneous evaluation of the presence and relative amounts of each target within the tissue sample. If desired, each probe can be detected individually using a wavelength filter to detect fluorescence from a particular quantum dot at the appropriate wavelength. In some embodiments, the signals are quantified by counting the number of pixels above background in each image.

In other embodiments, a composite spectral image showing the fluorescence from all quantum dots bound to the tissue is obtained using interferometric spectral imaging. The quantum dots are excited using ultraviolet light, e.g., 370 nm, and images of the quantum dots' fluorescence are obtained at various wavelengths, e.g., every 3-5 nm, across a broad spectrum, e.g., 450-800 nm, to produce a composite spectral image. Quantum dots emit fluorescence in a narrow Gaussian distribution, producing a spectral peak at the quantum dot's characteristic wavelength, e.g., Qd655 will produce a sharp spectral peak at 655 nm. This narrow distribution allows the composite spectral images to be unmixed using an appropriate software package and the signals from each quantum dot to be quantified. To unmix the composite spectral image, the light intensity of each pixel in each separate image is determined at each of the imaged wavelengths. Average background intensity is determined for the image, and any pixel with a light intensity comparable to the background is assigned a value of zero. A sharp increase in a pixel's light intensity is seen when a quantum dot at that location is imaged along its emission spectra, and the pixel is assigned a value of 1 for that wavelength. Quantum dot signals are quantified by counting the number of pixels in the image that were assigned a value of 1 at each quantum dot's characteristic wavelength. This procedure also can detect co-localized quantum dots, i.e., two different quantum dots (for example, Qd525 and Qd605) located at the same pixel position in the image. As the software steps through the sequential images, the light intensity at a particular pixel location increases at a first wavelength (e.g., 525 nm), indicating the presence of a first quantum dot that emits fluorescence at the first wavelength. As the software continues to step through the images, the light intensity at that pixel location will return to background, and then increase again at a second wavelength (e.g., 605 nm), indicating the presence of a second quantum dot that emits fluorescence at the second wavelength.

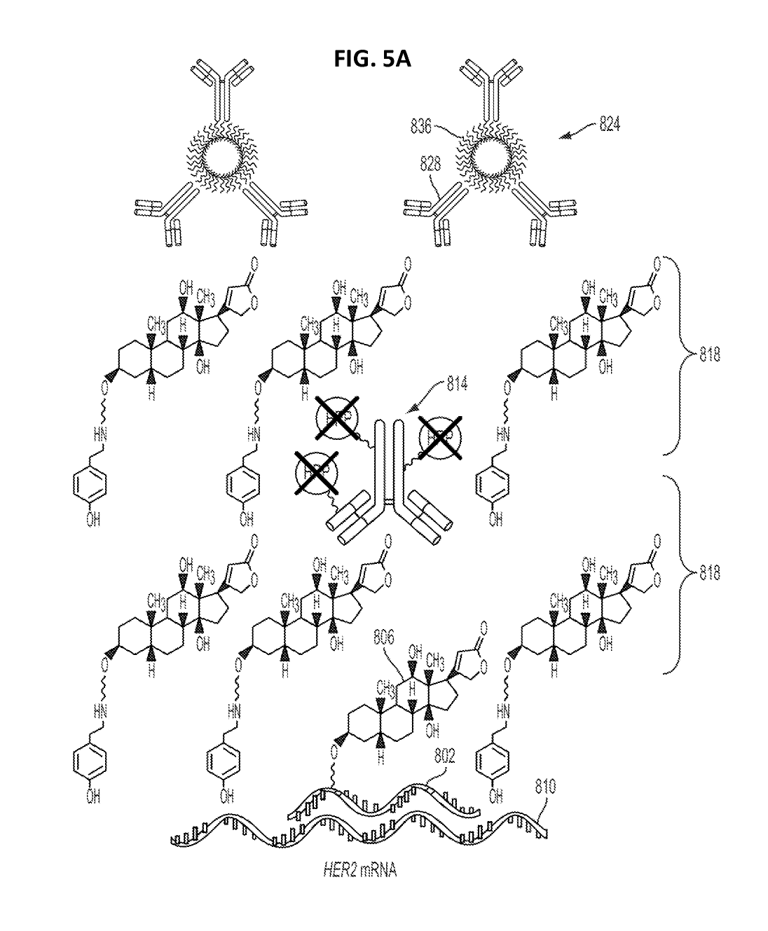

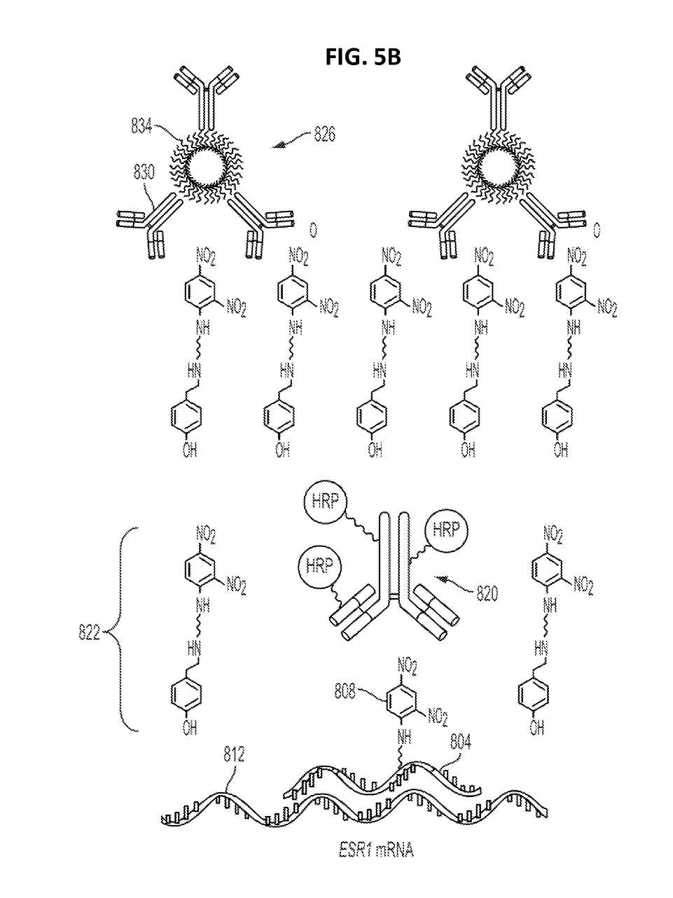

FIGS. 5A and 5B together illustrate one embodiment of a multiplexed RNA-ISH assay. A plurality anti-sense or sense strand RNA probes 802, 804 labeled with distinctive haptens 806, 808 are hybridized with a tissue sample, and bind to their respective gene targets 810, 812. Endogenous peroxidase is inactivated with a peroxidase inhibitor. In some embodiments, peroxidase is deactivated by addition of an excess of peroxide, and the sample is washed to remove excess peroxide. A first enzyme-conjugated anti-hapten monoclonal antibody 814 capable of recognizing and binding to hapten 806 is added to the tissue sample and allowed to react. In some embodiments, the enzyme is a peroxidase, such as horseradish peroxide (HRP) 816. The hapten 806 then is amplified by incubating the tissue sample in the presence of peroxide with a hapten-tyramide conjugate 818, which includes hapten 806. As hapten-tyramide conjugate 816 reacts with the enzyme, multiple hapten-tyramide conjugates 818 are deposited in the vicinity of probe 802. HRP 816 is inactivated (indicated by “X”) using a peroxidase inhibitor.

A second enzyme-conjugated anti-hapten monoclonal antibody 820 capable of recognizing and binding to hapten 808 is added to the tissue sample and allowed to react. The hapten 808 is amplified by incubating the tissue sample in the presence of peroxide with a tyramide-hapten conjugate 822, which includes hapten 808. As hapten-tyramide conjugate 822 reacts with the enzyme, multiple hapten-tyramide conjugates 822 are deposited in the vicinity of probe 804. If additional haptenylated probes are used, the steps are repeated to amplify each hapten.

After each hapten has been amplified, a mixture of anti-hapten monoclonal antibody-quantum dot conjugates 824, 826 are added to the tissue sample. Each conjugate 824, 826 includes antibodies 828, 830 capable of recognizing and binding to an individual hapten, e.g., hapten 806 or 808, respectively. Each conjugate 824, 826 also includes a distinct quantum dot 832, 834. For example, quantum dot 832 may be a Q655 that emits fluorescence at 655 nm, and quantum dot 834 may be a Qd525 that emits fluorescence at 525 nm.

In some embodiments, hapten-tyramide conjugates are used to detect micro RNA (miRNA or miR) using an RNA-ISH assay (see Example 6). MicroRNAs are small, non-coding RNAs that negatively regulate gene expression, such as by translation repression. For example, miR-205 regulates epithelial to mesenchymal transition (EMT), a process that facilitates tissue remodeling during embryonic development. However, EMT also is an early step in tumor metastasis. Down-regulation of microRNAs such as miR-205 may be an important step in tumor progression. For instance, expression of miR-205 is down-regulated or lost in some breast cancers. MiR-205 also can be used to stratify squamous cell and non-small cell lung carcinomas (J. Clin Oncol., 2009, 27(12):2030-7). Other microRNAs have been found to modulate angiogenic signaling cascades. Down-regulation of miR-126, for instance, may exacerbate cancer progression through angiogenesis and increased inflammation. Thus, microRNA expression levels may be indicative of a disease state.

### VI. Test Kits

Disclosed embodiments of the present disclosure include kits for carrying out various embodiments of the method of the invention. The kits include a hapten conjugate, such as a hapten-tyramide conjugate or hapten-tyramide derivative conjugate as disclosed herein. In some embodiments, the kit further includes a peroxide solution, e.g., a hydrogen peroxide solution. In a particular embodiment, the kit includes an HQ-tyramide conjugate and a hydrogen peroxide solution.

In some embodiments, the kit includes a plurality of hapten conjugates, such as hapten-tyramide conjugates and/or hapten-tyramide derivative conjugates, as disclosed herein. Such kits may be particularly useful for multiplexed detection of multiple targets in a sample. In certain embodiments, the kit further may include one or more hapten-labeled probes capable of binding to one or more targets in a sample.

In particular embodiments, the kit further may include an anti-hapten antibody, an anti-hapten antibody-peroxidase conjugate, an antibody-label conjugate wherein the antibody is capable of recognizing and binding to an anti-hapten antibody, an anti-hapten antibody-label conjugate, or any combination thereof. The label can be any detectable label capable of being conjugated to an antibody. Detectable labels include, for example, enzymes that can be detected in chromogenic assays and quantum dots that can be detected in fluorescence assays.

In some embodiments, the kit additionally may contain suitable reagents for detecting the label. For example, if the label is HRP, the kit may include reagents for performing a 3,3′-diaminobenzidine (DAB) assay.

### VII. EXAMPLES

The following examples are provided to illustrate certain specific features of working embodiments and general protocols. The scope of the present invention is not limited to those features exemplified by the following examples.

### Example 1

### Hapten-dPEG®₈-Tyramide Synthesis

This example illustrates one method suitable for forming hapten-linker-tyramide conjugates.

### General Procedure for Synthesizing Hapten-dPEG®₈-Tyramide Conjugates

The synthesis shown in Scheme 1 (Section IV) was used to prepare hapten-dPEG %-tyramide conjugates for haptens other than HQ (3-hydroxyquinoxaline), which was prepared by a different synthesis described below. The haptens included BD (benzodiazepine), BF (benzofurazan), DABSYL (4-(dimethylamino)azobenzene-4′-sulfonamide), DCC (7-(diethylamino)coumarin-3-carboxylic acid), DIG (digoxigenin), DNP (dinitrophenyl), FITC (fluorescein isothiocyanate), NCA (nitrocinnamic acid), NP (nitropyrazole), PPT (Podophyllotoxin), Rhod (rhodamine), ROT (rotenone), and TS (thiazolesulfonamide). Synthesis began with generating the hapten NHS ester utilizing N,N′-dicyclohexyl-carbodiimide as the coupling agent. The urea byproduct was filtered off, and the active ester was used without further purification. The active esters were then coupled to the dPEG®₈ amino acid (Quanta BioDesign, Ltd., Powell, Ohio) under basic conditions, and the product was purified via flash chromatography. The NHS esters of the carboxy-dPEG®₈-haptens were generated using N,N′-dicyclohexyl-carbodiimide as detailed above. Treatment with a slight excess of tyramine followed by flash chromatography afforded the hapten-dPEG®₈-tyramides.

### Individual Tyramide Conjugates

Note: In all examples the hapten dPEG®₈ NHS esters were synthesized as previously detailed.

### N-(30-(4-Hydroxyphenyl)-27-oxo-3,6,9,12,15,18,21,24-octaoxa-28-azatriacontyl)benzo[c][1,2,5]oxadiazole-5-carboxamide (BF)

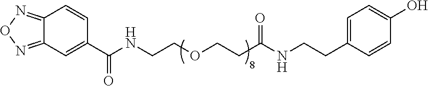

The active ester intermediate (1.26 mmol) and tyramide (1.64 mmol) were taken in 3 mL dry DMF and allowed to stir under dry nitrogen for sixteen hours. The residue was concentrated, dissolved in minimal DCM and purified by flash chromatography affording 765 mg of the tyramide product (86%) as a thick oil.

### 2,4-DinitrophenyhdPEG %-carboxytyramide (DNP)

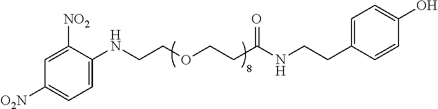

The active ester intermediate (1.31 mmol) and tyramide (1.31 mmol) were taken in 4 mL dry DMF and allowed to stir under dry nitrogen for sixteen hours. The residue was concentrated, dissolved in minimal DCM and purified by flash chromatography affording 837 mg of the tyramide product (88%) as a thick yellow oil.

### 7-(Diethylamino)-N-(30-(4-hydroxyphenyl)-27-oxo-3,6,9,12,15,18,21,24-octaoxa-28-azatriacontyl)-2-oxo-2H-chromene-3-carboxamide (DCC)

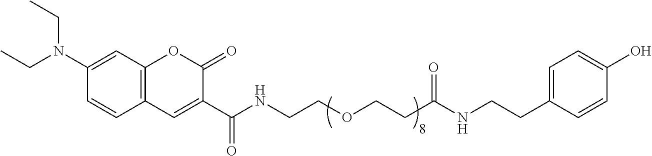

The active ester intermediate (1.26 mmol) and tyramide (1.26 mmol) were taken in 3 mL dry DMF and allowed to stir under dry nitrogen for sixteen hours. The residue was concentrated, dissolved in minimal DCM and purified by flash chromatography affording 508 mg of the tyramide product (71%) as a thick yellow oil.

### 4,5-Dimethoxy-2-nitrocinnamic-dPEG®₈-carboxytyramide (NCA)

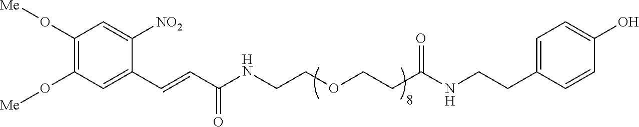

The active ester intermediate (1.45 mmol) and tyramide (1.60 mmol) were taken in 3 mL dry DMF and allowed to stir under dry nitrogen for sixteen hours. The residue was concentrated, dissolved in minimal DCM and purified by flash chromatography affording 958 mg of the tyramide product (83%) as a thick oil.

### 2-Acetamido-4-methyl-5-thiazolesulfonamide-dPEG®₈-carboxytyramide (TS)

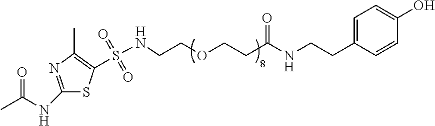

The active ester intermediate (2.27 mmol) and tyramide (2.72 mmol) were taken in 5 mL dry DMF and allowed to stir under dry nitrogen for sixteen hours. The residue was concentrated, dissolved in minimal DCM and purified by flash chromatography affording 1.255 g of the tyramide product (71%) as a thick oil.

### 2-(2-(2-oxo-2,3-dihydro-1H-benzo[b][1,4]diazepin-4-yl)phenoxy)-(27-oxo-3,6,9,12,15,18,21,24-octaoxaoctacosyl)carboxy-tyramide (BD)

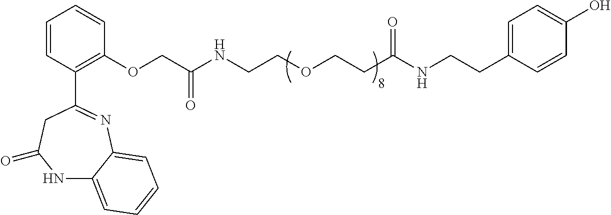

The active ester intermediate (0.706 mmol) and tyramide (0.706 mmol) were taken in 3 mL dry DMF and allowed to stir under dry nitrogen for sixteen hours. The residue was concentrated, dissolved in minimal DCM and purified by flash chromatography affording 536 mg of the tyramide product (91%) as a thick oil.

### 5-Nitro-3-pyrazole-dPEG®₈-carboxytyramide (NP)

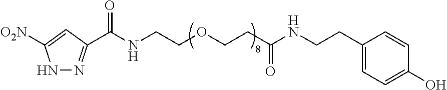

The active ester intermediate (1.27 mmol) and tyramide (1.33 mmol) were taken in 3 mL dry DMF and allowed to stir under dry nitrogen for sixteen hours. The residue was concentrated, dissolved in minimal DCM and purified by flash chromatography affording 730 mg of the tyramide product (79%) as a thick oil.

### Pyrazopodophyllamide-dPEG®₈-carboxytyramide (PPT)

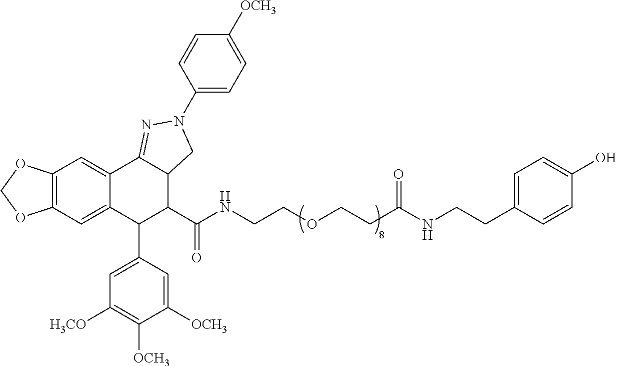

The active ester intermediate (7.65 μmol) and tyramide (7.29 μmol) were taken into dry DMF at a concentration of 10 mg/mL and allowed to stir under dry nitrogen for sixteen hours. The reaction mixture was purified by semi-preparative HPLC affording 4.4 μmol of the tyramide product (61%) as a thick oil.

### Rotenone isoxazolinamide-dPEG®₈-carboxytyramide (ROT)

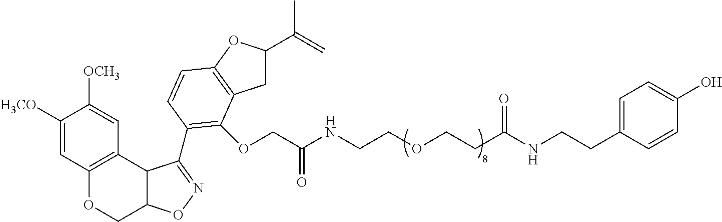

The active ester intermediate (7.65 mmol) and tyramide (7.29 mmol) were taken into dry DMF at a concentration of 10 mg/mL and allowed to stir under dry nitrogen for sixteen hours. The reaction mixture was purified by semi-preparative HPLC affording 5.3 μmol of the tyramide product (73%) as a thick oil.

### 4-(Dimethylamino)azobenzene-4′-sulfonamide-dPEG®-carboxytyramide (DABSYL)

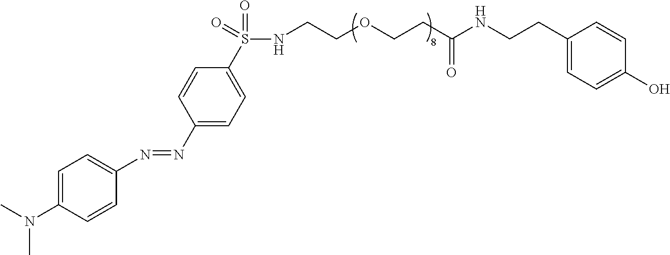

The active ester intermediate (7.65 μmol) and tyramide (7.29 μmol) were taken into dry DMF at a concentration of 10 mg/mL and allowed to stir under dry nitrogen for sixteen hours. The reaction mixture was purified by semi-preparative HPLC affording 6.3 μmol of the tyramide product (87%) as a thick oil.

### HO-dPEG®₈-Tyramide

### 3-Hydroxy-N-(30-(4-hydroxyphenyl)-27-oxo-3,6,9,12,15,18,21,24-octaoxa-28-azatriacontyl)quinoxaline-2-carboxamide (HQ)

To a solution of 3-hydroxyquinoxaline-2-carboxylic acid (11.55 mmol, 1.0 eq.) in 10 mL of dry DMF was added EDAC (17.33 mmol, 1.5 eq.) and N-hydroxysuccinimide (17.33 mmol, 1.5 eq.) and the reaction stirred 16 hours under dry nitrogen. The reaction was filtered through a sintered glass funnel and the yellow precipitate washed 2 times with 2 mL DMF then dried under vacuum to give 3.25 g (11.3 mmol, 98%) of the active ester 1 as a yellow solid.

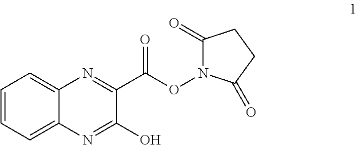 [SMILES: O=C(ON1C(=O)CCC1=O)c1nc2ccccc2nc1O]

To a solution of 2,5-dioxopyrrolidin-1-yl 3-hydroxyquinoxaline-2-carboxylate 1 (2.1 mmol, 1.0 eq.) in 5 mL of dry DMF was added amino-dPEG®₈-carboxylic acid (2.3 mmol, 1.1 eq.) and triethylamine (3.45 mmol, 1.5 eq.) and the reaction stirred 3 hours under dry nitrogen. The reaction was concentrated under vacuum and taken in minimal DCM. Automated flash chromatography eluting with 10-20% MeOH/DCM containing 0.5% AcOH afforded 1.21 g (1.97 mmol, 94%) of the amino acid 2 as a yellow oil.

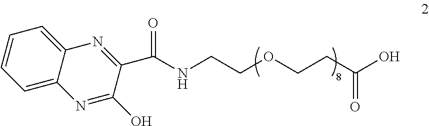

To a solution of 1-(3-hydroxyquinoxalin-2-yl)-1-oxo-5,8,11,14,17,20,23,26-octaoxa-2-azanonacosan-29-oic acid 2 (2.18 mmol, 1.0 eq.) in 10 mL of dry DCM was added 1.0 M DCC in DCM (3.27 mmol, 1.5 eq.) and N-hydroxysuccinimide (3.27 mmol, 1.5 eq.) and the reaction stirred 16 hours under dry nitrogen. The reaction was filtered through a sintered glass funnel to remove the urea byproduct and the residue dried under vacuum to give 1.35 g of the active ester 3, which was used without further purification.

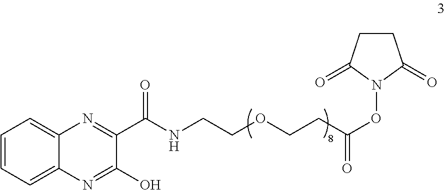

To a solution of 2,5-dioxopyrrolidin-1-yl 1-(3-hydroxyquinoxalin-2-yl)-1-oxo-5,8,11,14,17,20,23,26-octaoxa-2-azanonacosan-29-oate 3 (0.49 mmol, 1.0 eq.) in 5 mL of dry DMF was added tyramine (0.54 mmol, 1.1 eq.) and the reaction stirred 18 hours under dry nitrogen. The reaction was diluted with DCM then 2 times with saturated sodium bicarbonate then 2 times with brine and the organic phase concentrated under vacuum and taken in minimal DCM. Automated flash chromatography eluting with 5-20% MeOH/DCM afforded 0.312 g (0.426 mmol, 86%) of the hapten-tyramide conjugate 4 as a thick yellow oil.

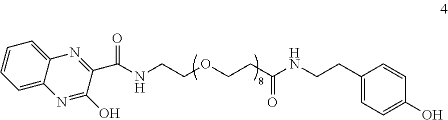

### 3-hydroxy-N-(30-(4-hydroxyphenyl)-27-oxo-3,6,9,12,15,18,21,24-octaoxa-28-azatriacontyl)quinoxaline-2-carboxamide

The synthesized hapten-tyramide conjugates were characterized by HPLC, UV/VIS, and mass spectroscopy. HPLC was performed with an injection volume of 8 μL and a run time of 15.0 minutes, with absorbance measured at 254 nm. A Waters C18 X-Bridge 4.6×100 mm (5μ) column running a water/acetonitrile gradient was used. UV/VIS spectra were obtained over a range of 200-600 nm. Mass spectroscopy was performed on a JEOL AccuTOF using ESI with an acquired m/z range of 100-3000. The structures of particular hapten-tyramide conjugates synthesized and used in subsequent examples are shown below in Table 3.

TABLE 3

| Hapten | Structure |
|---|---|
| BD | 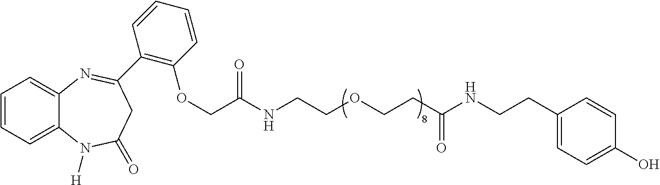 |
| BF | 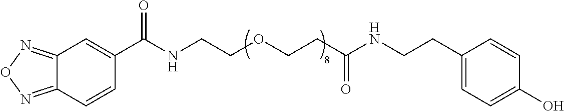 |
| DAB | 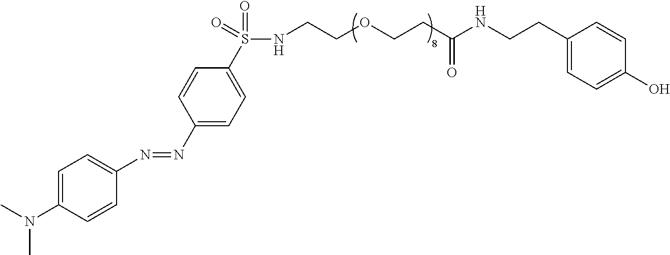 |
| DCC | 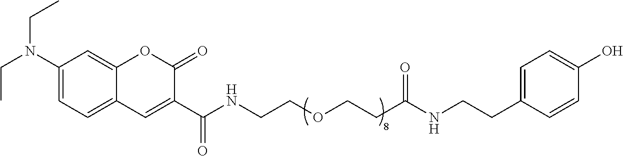 |
| DIG | 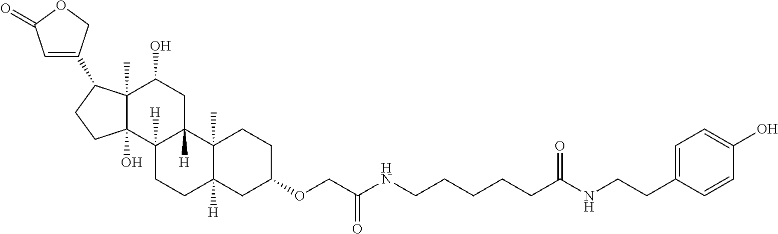 |
| DNP | 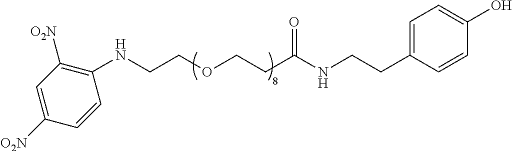 |
| HQ | 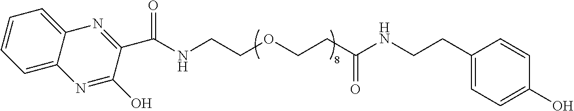 |
| NCA | 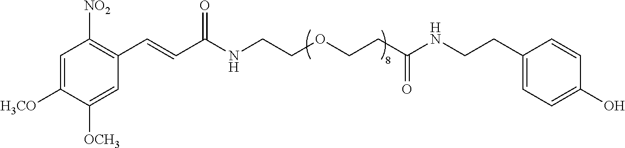 |
| NP | 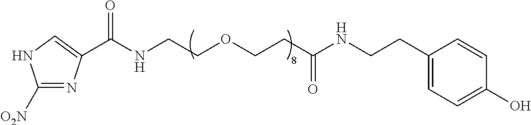 |
| PPT | 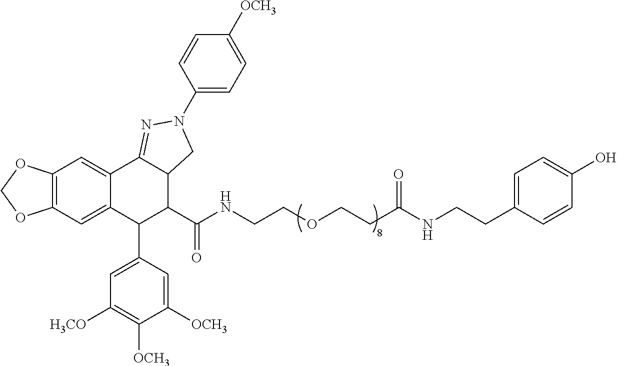 |
| ROT | 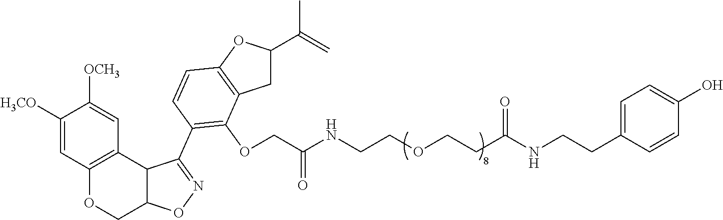 |
| TS | 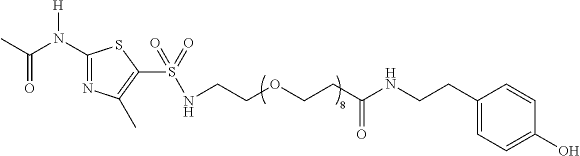 |

### Example 2

### Evaluation of bcl2 (124) Antibody on Tonsil Tissue for the Comparison of Tyramide-Hapten Conjugates

This example demonstrates the visualization of bcl2 (124) antibody on tonsil tissue using tyramide-hapten conjugates. Haptens were conjugated to tyramine via a polyethylene glycol linker to form a hapten-dPEG®₈-tyramide conjugate as described in Example 1. Haptens evaluated included BD, BF, DABSYL, DCC, DIG, DNP, FITC, HQ, NCA, NP, PPT, Rhod, ROT, and TS.

Slides containing tonsil tissue sections were developed using a standard protocol for an automated stainer (BenchMark® XT, Ventana Medical Systems, Inc, (VMSI) Tucson, Ariz.). A typical automated protocol is as follows.

The paraffin-coated tissue on the slides was heated to 75° C. for 8 minutes and treated once with EZPrep (VMSI #950-102), volume adjusted at 75° C. before application of the Liquid Cover Slip (LCS, VMSI #650-010). After another 8-minute incubation at 75° C., the slide was rinsed and EZPrep volume was adjusted, followed with LCS to deparaffinize the tissue. The slides were cooled to 37° C. and incubated for 4 minutes. The slides were thoroughly rinsed with EZPrep, followed by application of LCS. The slides were heated to 95° C. for 8 minutes, followed by application of LCS. The slides were then heated to 100° C. and incubated for 4 minutes. Every 4 minutes, for 24 minutes, cell condition solution (CC1, VMSI #950-124) and LCS were applied in order to prevent slide drying. After 2 rinses with reaction buffer (VMSI #950-300), 100 μL of UV Inhibitor (a component of the VMSI ultraView DAB Detection Kit #760-500) was applied to the slide sand incubated for 4 minutes. The slides were rinsed once with reaction buffer before the application of 100 μL of bcl2 (124) antibody (VSMI #760-4240) for 16 minutes at 37° C. The slides were rinsed 3 times with reaction buffer before the addition of 100 μL of blocking solution (10% dextran sulfate sodium salt (avg. MW 10K), 2.5 M sodium chloride, 1% BSA, 0.1% cold fish skin gelatin, 0.1% Triton® X-100, 0.05% Tween® 20, 0.1% Proclin® 300) and 100 μL of ultraView HRP universal multimer (a component of the VMSI ultraView DAB Detection Kit #760-500). The 2 reagents were co-incubated at 37° C. for 20 minutes.

The slides were rinsed with reaction buffer four times before 100 μL of the tyramide hapten conjugates were manually applied to the slide. Tyramide-hapten conjugates were diluted to 55 μM and 5.5 μM in tyramide amplification diluent (0.75 mM sodium stannate, 40 mM boric acid, 10 mM sodium tetraborate decahydrate, and 30 mM sodium chloride). After the manual applications were completed, 100 μL of the ultraView H₂O₂ was applied to the slides and incubated for 12 minutes at 37° C. After washing the slides 3 times in reaction buffer, 100 μL of the blocking solution and 100 μL of a 5 μg/mL solution of the respective mouse anti-hapten monoclonal antibody conjugated to HRP were co-incubated for 8 minutes at 37° C. The HRP conjugates were diluted in 0.1 M PBS buffer, pH 7.2, with 13.5 mg/mL hydrolyzed casein. After 4 rinses with reaction buffer, 100 μL of both the ultraView DAB and ultraView H₂O₂ were applied to the slide and co-incubated for 8 minutes with LCS at 37° C. The slides were rinsed once in reaction buffer before 100 μL of the UltraView Copper was applied to the slide and incubated for 4 minutes at 37° C. The slides then underwent 2 rinses with reaction buffer before counterstaining with Hematoxylin II (VMSI #750-2021) which was incubated on the slide for 4 minutes with LCS. After 2 rinses with reaction buffer, the bluing reagent (VMSI #760-2037) was applied and incubated for 4 minutes for the counterstain to be complete. The slides were removed from the instrument and treated to a detergent wash before manual application of a solid cover slip.

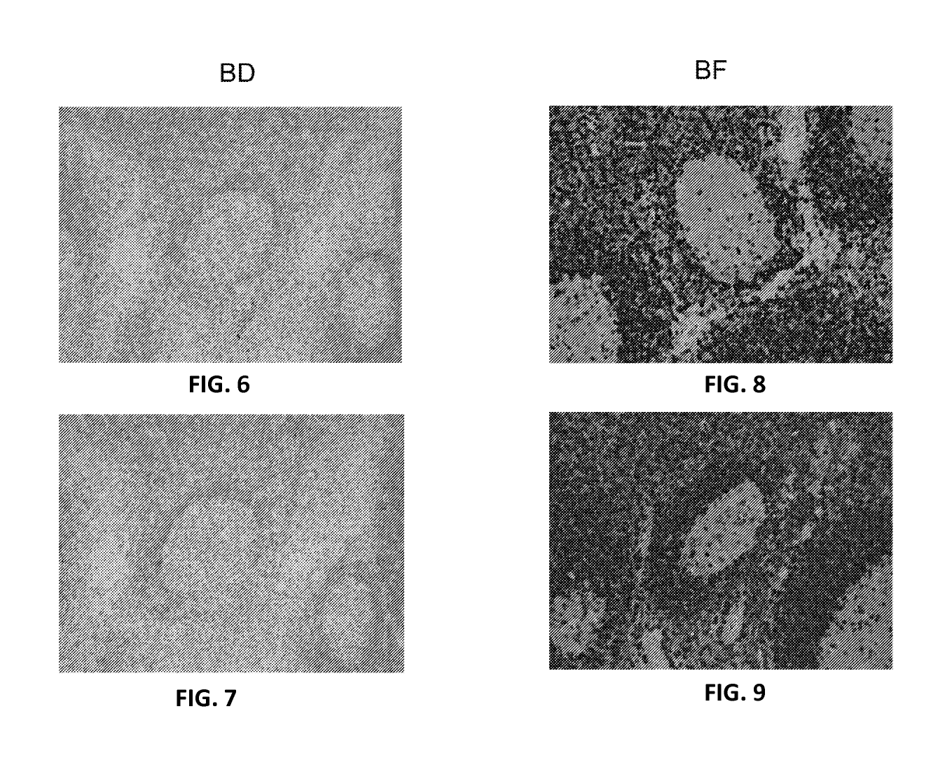

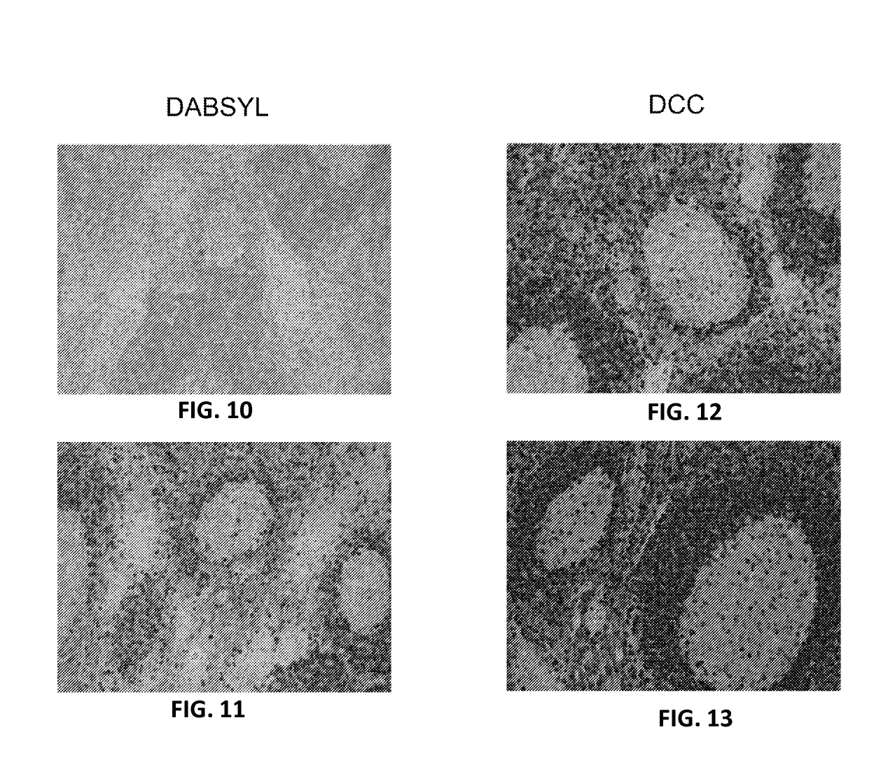

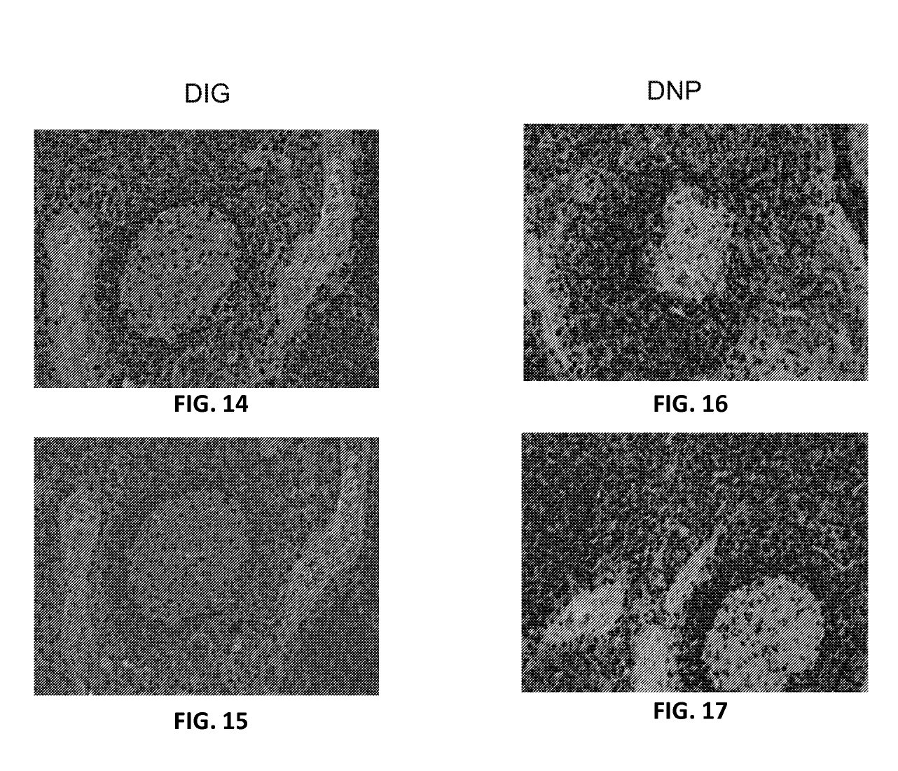

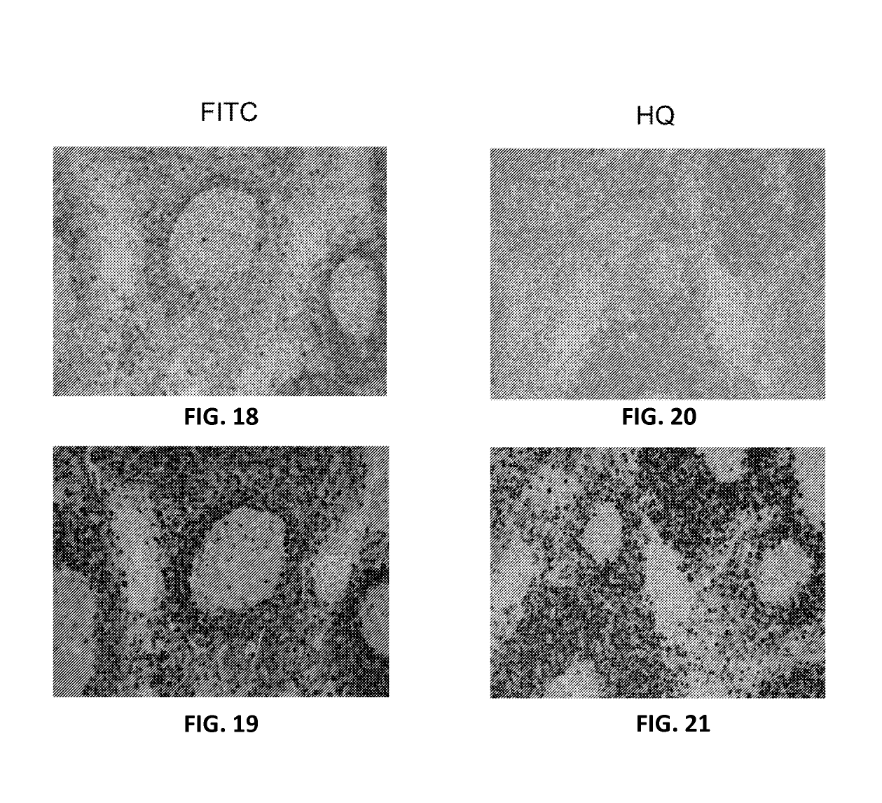

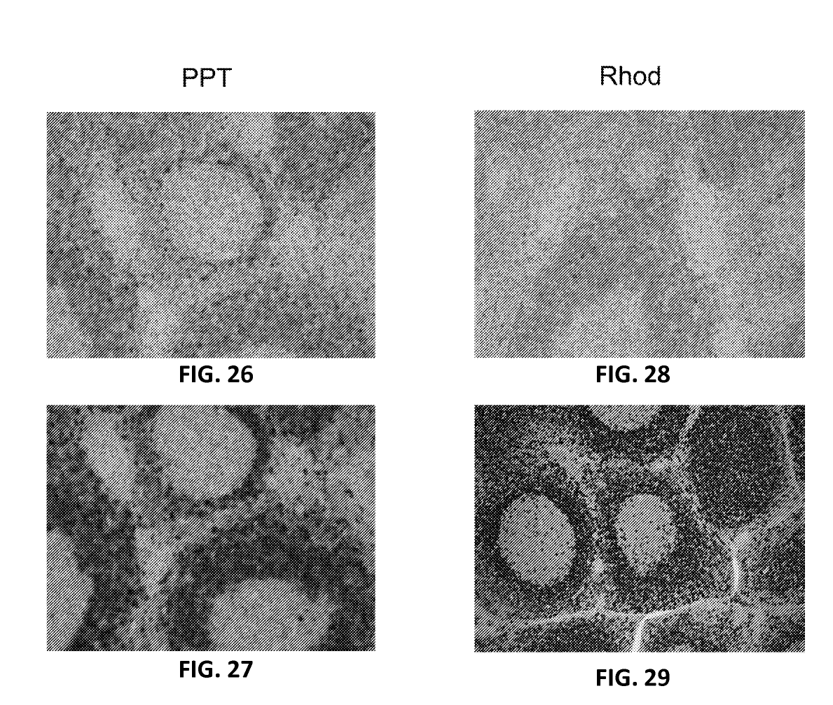

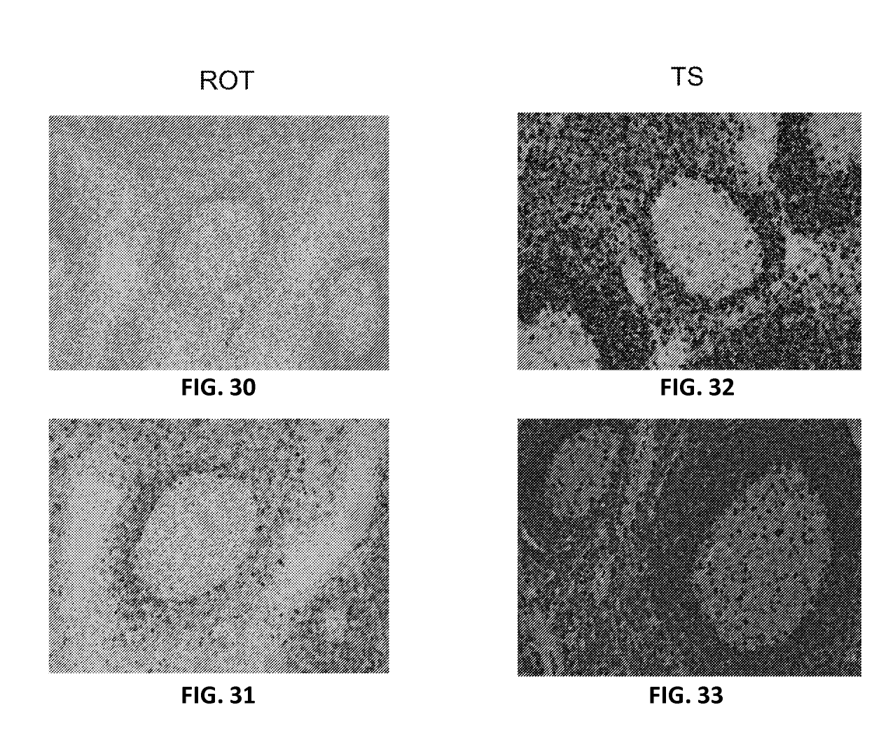

The slides were viewed through a brightfield microscope. Photographs of the slides are shown in FIGS. 6-33. The results shown in Table 4 include a subjective score of the signal strength (e.g., the intensity of the staining) on a scale of 1-4, with 4 being the most intensely stained. The background (BG) score and signal:noise ratio also are provided.

TABLE 4

| Conjugate Conc. | | TA-Hapten | Score | BG score | Signal:Noise |
|---|---|---|---|---|---|
| 5.5 | uM | HQ | 1 | 0.5 | 2 |
| 55 | uM | HQ | 4 | 0.25 | 16 |
| 5.5 | uM | PPT | 2 | 0.5 | 4 |
| 55 | uM | PPT | 3.5 | 0.75 | 5 |
| 5.5 | uM | BD | 1 | 0.5 | 2 |
| 55 | uM | BD | 2 | 0.25 | 8 |
| 5.5 | uM | DIG | 4 | 3 | 1 |
| 55 | uM | DIG | 4 | 3.75 | 1 |
| 5.5 | uM | DNP | 4 | 1.5 | 2 |
| 55 | uM | DNP | 4 | 2 | 2 |
| 5.5 | uM | DCC | 4 | 1.5 | 2 |
| 55 | uM | DCC | 4 | 3 | 1 |
| 5.5 | uM | NP | 4 | 1 | 4 |
| 55 | uM | NP | 4 | 3.5 | 1 |
| 5.5 | uM | Rhodamine | 2 | 0.5 | 4 |
| 55 | uM | Rhodamine | 4 | 0.25 | 16 |
| 5.5 | uM | NCA | 0.5 | 0.25 | 2 |
| 55 | uM | NCA | 4 | 1.5 | 2 |
| 5.5 | uM | FITC | 4 | 1 | 4 |
| 55 | uM | FITC | 4 | 3 | 1 |
| 5.5 | uM | TS | 4 | 2.5 | 2 |
| 55 | uM | TS | 4 | 3.75 | 1 |
| 5.5 | uM | BF | 4 | 2.75 | 1 |
| 55 | uM | BF | 3.5 | 2 | 2 |
| 5.5 | uM | DABSYL | 1 | 0.5 | 2 |
| 55 | uM | DABSYL | 4 | 0.25 | 16 |
| 5.5 | uM | ROT | 2 | 0.5 | 4 |
| 55 | uM | ROT | 3.75 | 0.25 | 15 |

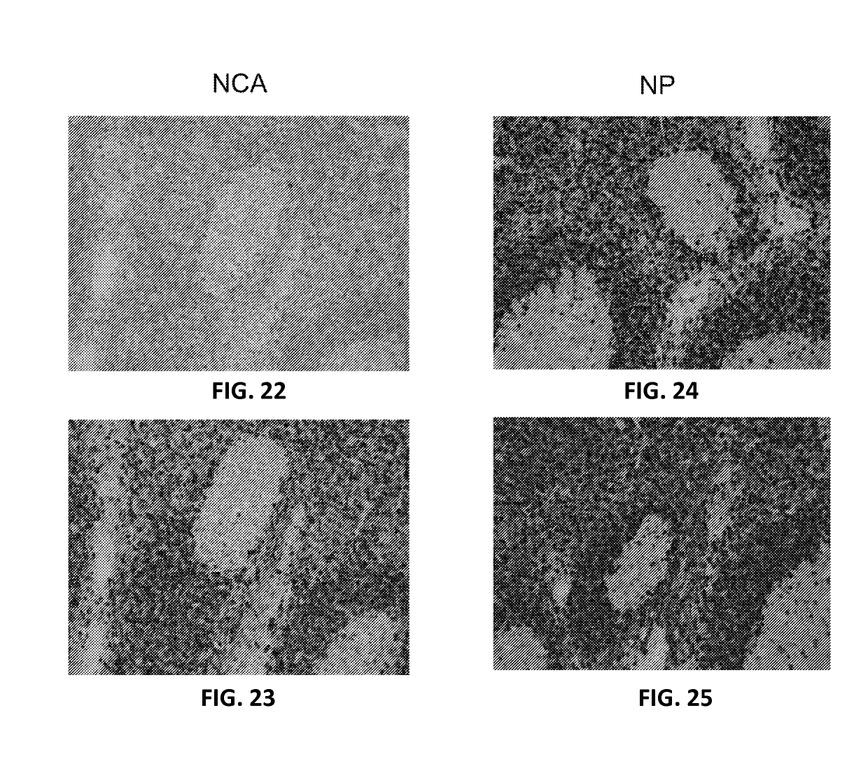

Conjugates that provided exemplary results included the DABSYL-, HQ-, rhodamine-, and rotenone-tyramide conjugates (FIGS. 8-9, 20-21, and 28-31). Where a darker stain is preferred, NCA- and NP-tyramide conjugates also produced superior results (FIGS. 22-25). The staining darkness can be adjusted, for example, by adjusting incubation times of the tyramide-hapten conjugates, adjusting the primary antibody concentration (i.e., the bcl2 (124) antibody in this example), and/or adjusting the primary antibody incubation time.

### Example 3

### Comparison of Native Anti-Hapten Signals and Hapten-Tyramide Conjugate Signals in an mRNA-ISH Assay

This example compares the signals obtained in an mRNA-ISH assay when haptens are directly bound to a probe and the signals obtained when tyramide signal amplification is performed using hapten-tyramide conjugates. Haptens were conjugated to tyramine via a polyethylene glycol linker to form a hapten-dPEG®₈-tyramide conjugate as described in Example 1. BD, BF, DABSYL, DCC, DIG, DNP, HQ, NCA, NP, PPT, ROT, and TS haptens were evaluated.

### Native Anti-Hapten Signal Determination

Dot Blot Construction.

Three one microliter drops of sense strand or anti-sense strand ACTB (beta-actin) RNA at different concentrations suspended in Genorama spotting solution were spotted onto distinct areas of Asper SA-1 microarray slides (Asper Biotech Ltd., Tartu, Estonia), and the slides were allowed to dry at room temperature. RNA was cross-linked to the slides using 300 mJ of UV radiation.

Dot Blot Hybridization.

ACTB anti-sense riboprobes chemically labeled with different haptens using Mirus linker arms were prepared as directed by the manufacturer (Mirus Bio LLC, Madison, Wis.). One hundred nanograms of each probe was suspended in 1 mL of Ribohybe™ (VMSI #760-104) solution and placed in distinct dispensers. RNA was spotted onto Asper SA-1 microarray slides, and UV crosslinked. Prepared dot blot slides were loaded onto the Discovery® XT instrument (VMSI) and one drop (100 μL) of a haptenylated antisense ACTB riboprobe was dispensed onto a slide, denatured at 80° C. for 8 min, and hybridized at 65° C. for 6 hrs. Following hybridization, the slide was washed 3 times using 0.1×SSC (sodium chloride/sodium citrate buffer, VMSI #950-110) at 75° C. for 8 min. Each uniquely haptenylated probe was detected using 5 μg of cognate native anti-hapten antibody followed by a biotinylated goat anti-mouse polyclonal antibody (VMSI #213-2194) and streptavidin conjugated to quantum dot (Qd) 655 (Invitrogen, Carlsbad, Calif.). The slides were dehydrated using gradient alcohols and coverslipped.

Dot Blot Signal Quantification.

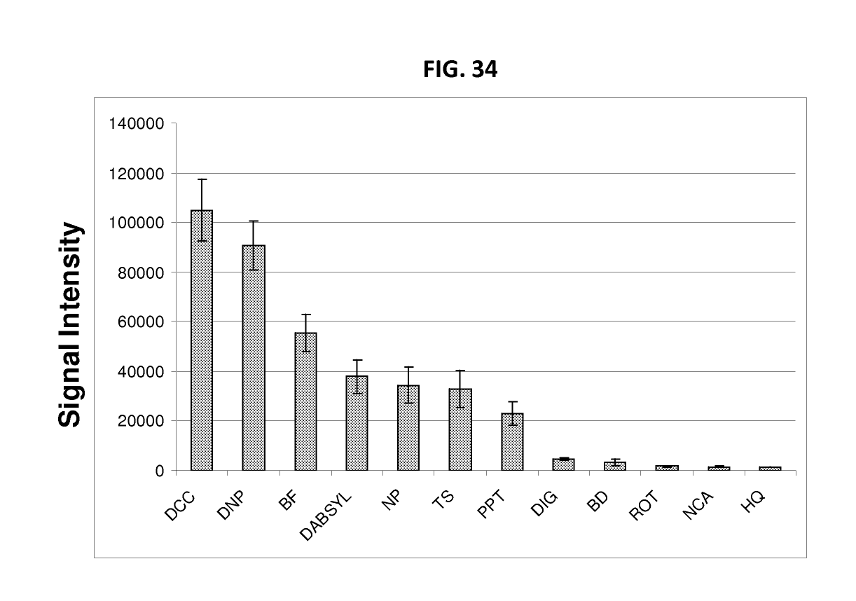

Dot blot slides were analyzed using a Zeiss fluorescent microscope fitted with a Spectral Imaging camera (Applied Spectral Imaging (ASI), Vista, Calif.). Images for each of the three sense (experimental) and anti-sense (negative control) spots per dot blot slide were captured using a 40× objective and ASI software package. The value of each dot's fluorescent signals, generated from the Qd655 conjugated antibody, was captured by exporting the raw 655-nm spectral data for each pixel to an Excel spreadsheet; signal for each pixel was averaged for each distinct dot and 95% confidence intervals were determined for the spots. Background was determined using signals for the negative control anti-sense spots. On all slides background was negligible and did not significantly contribute to experimental signals. Data was plotted for each hapten/native anti-hapten pair (FIG. 34). The variability in the signal suggests a range of native-anti-hapten antibody detection efficiencies where DCC>DNP>BF>DABSYL>NP>TS>PPT>DIG>BD>ROT>NCA>HQ.

Tissue Hybridization.

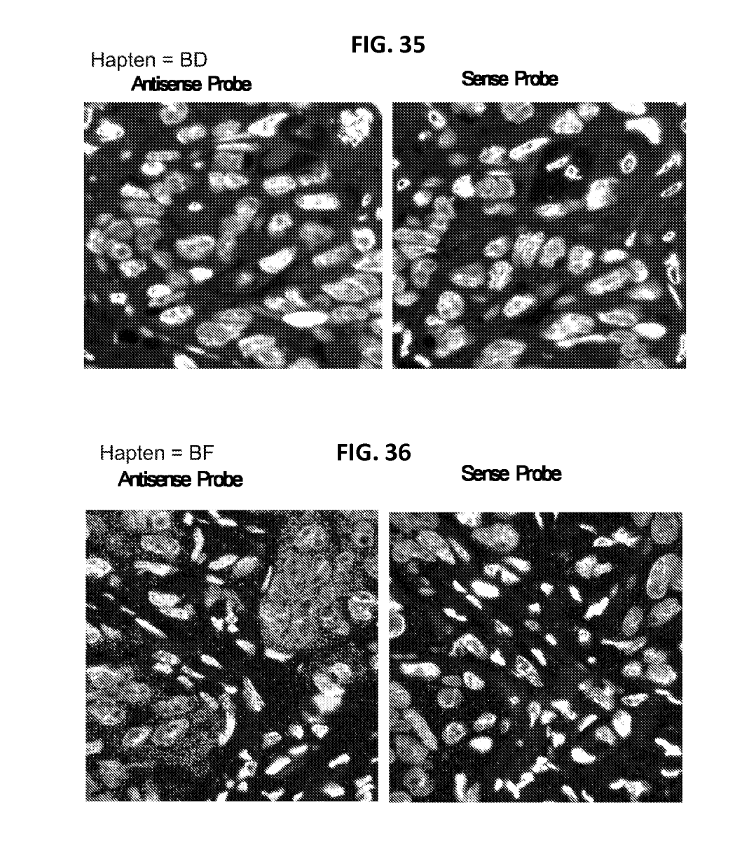

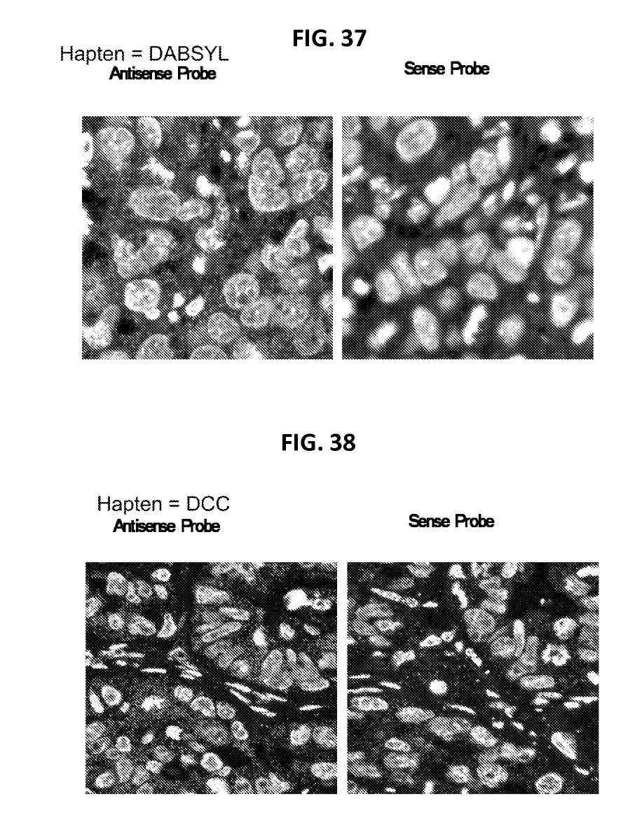

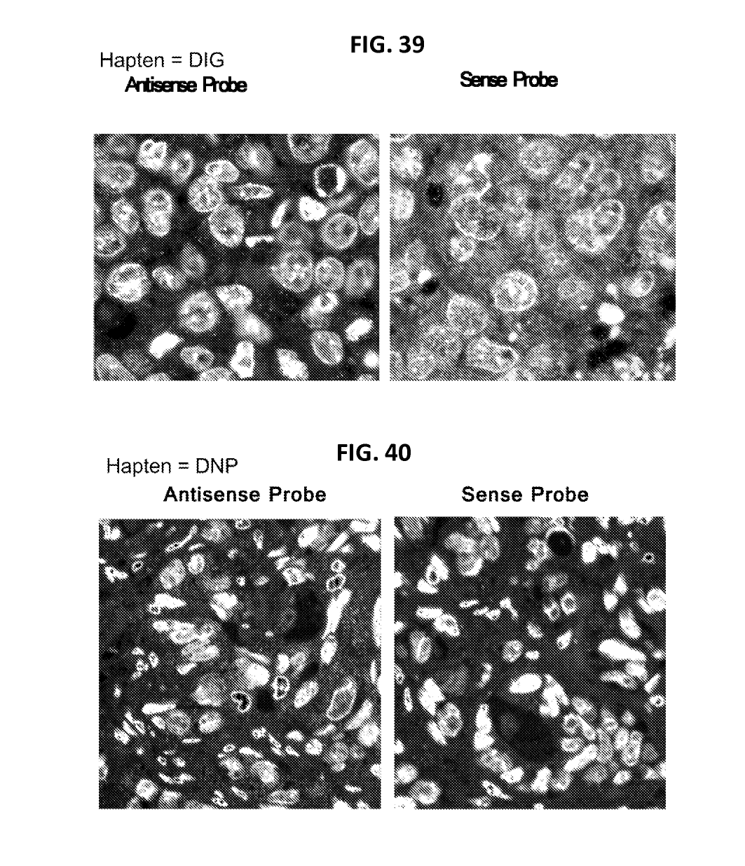

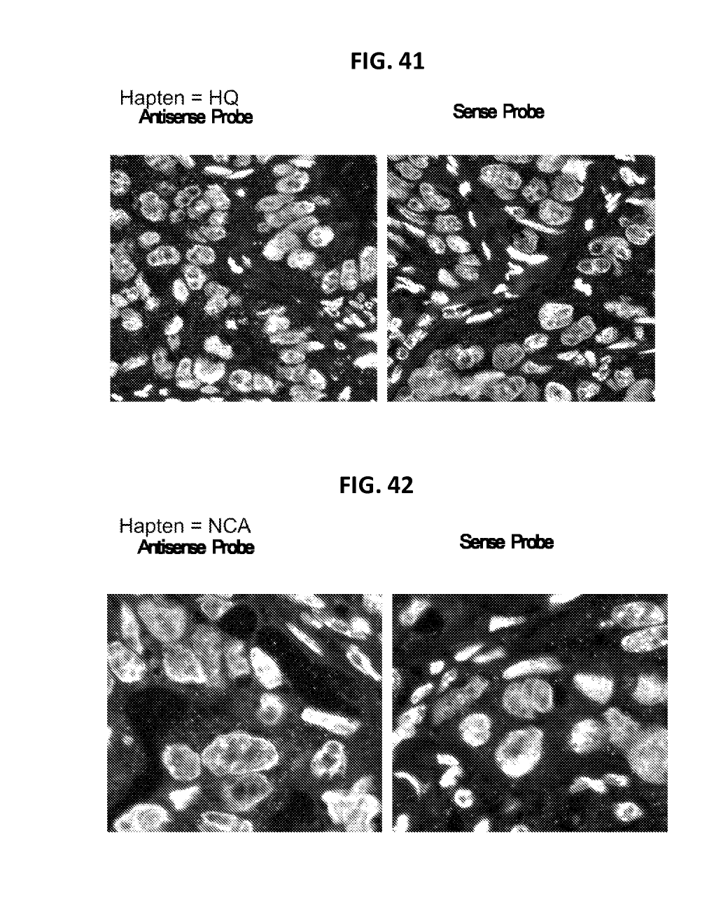

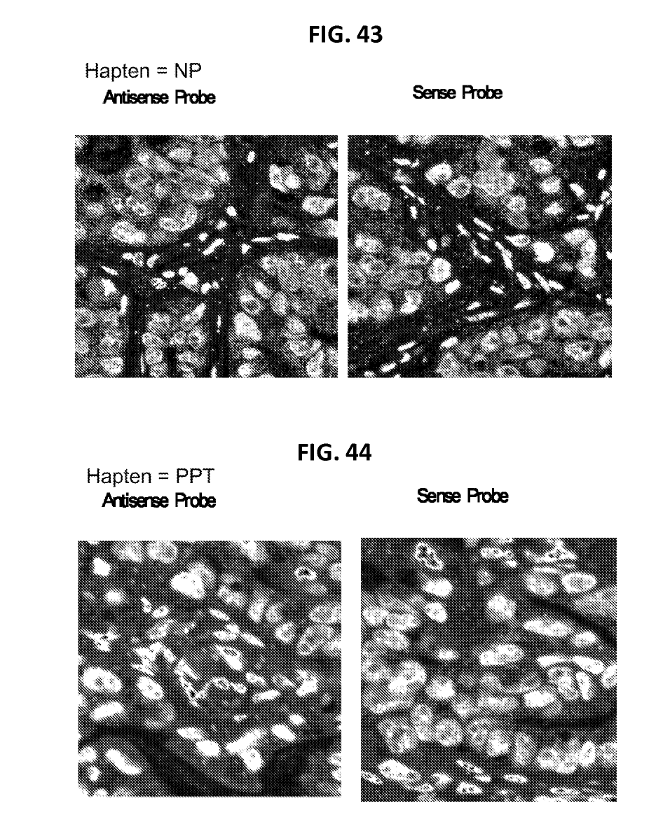

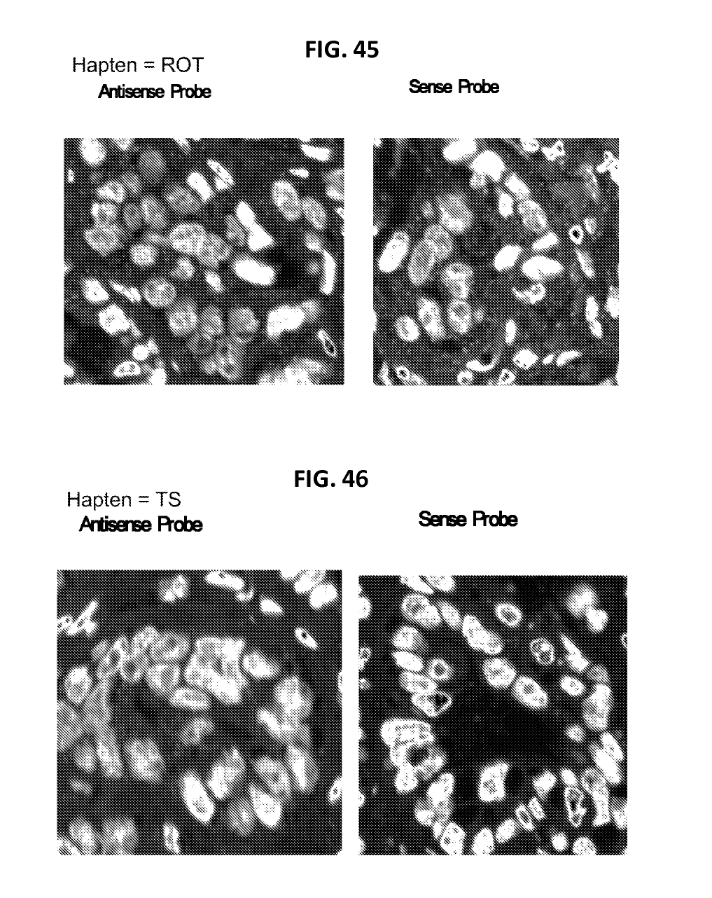

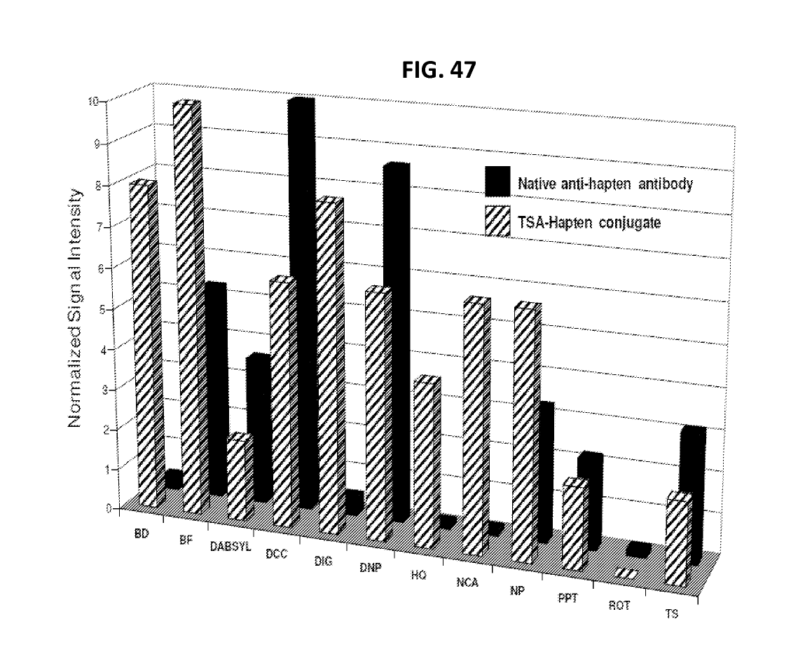

Formalin-fixed, paraffin-embedded Calu-3 xenograft tissue mounted on Superfrost slides was de-paraffinized and antigen retrieved using RiboClear (VMSI #760-4125) denaturant, RiboCC VMSI #760-107) reagent, and protease 3 (VMSI #760-2020). Following retrieval, one drop (100 μL) of a haptenylated anti-sense or sense strand ACTB probe was dispensed onto a slide, denatured at 80° C. for 8 min, and hybridized at 65° C. for 6 hrs. Following hybridization slides were washed 3 times using 0.1×SSC at 75° C. for 8 min; each uniquely haptenylated probe was detected using 5 μg of cognate native anti-hapten antibody followed by a biotinylated goat anti-mouse polyclonal antibody and streptavidin conjugated to Qd655. Slides were counterstained using DAPI (VMSI #760-4196). The slides were dehydrated using gradient alcohols and coverslipped. The DAPI and 655-nm QDot™ emission signals were imaged using an Olympus fluorescent microscope fitted with a Spectral Imaging camera (Applied Spectral Imaging (ASI) Vista, Calif.) (FIGS. 35-46). The sense strand was used as a negative control. Any staining observed with the sense strand would indicate how much background or non-specific staining could be attributed to the detection system alone.

Tissue Signal Quantification.

The QDot™ 655 nm fluorescent emission signals were used to rank the images. The ranking was done by two blinded readers using a relative signal:noise (anti-sense:sense probe) intensity scale (0-10) (FIG. 47).

### Hapten-Tyramide Conjugate Signal Determination

Tissue Staining.

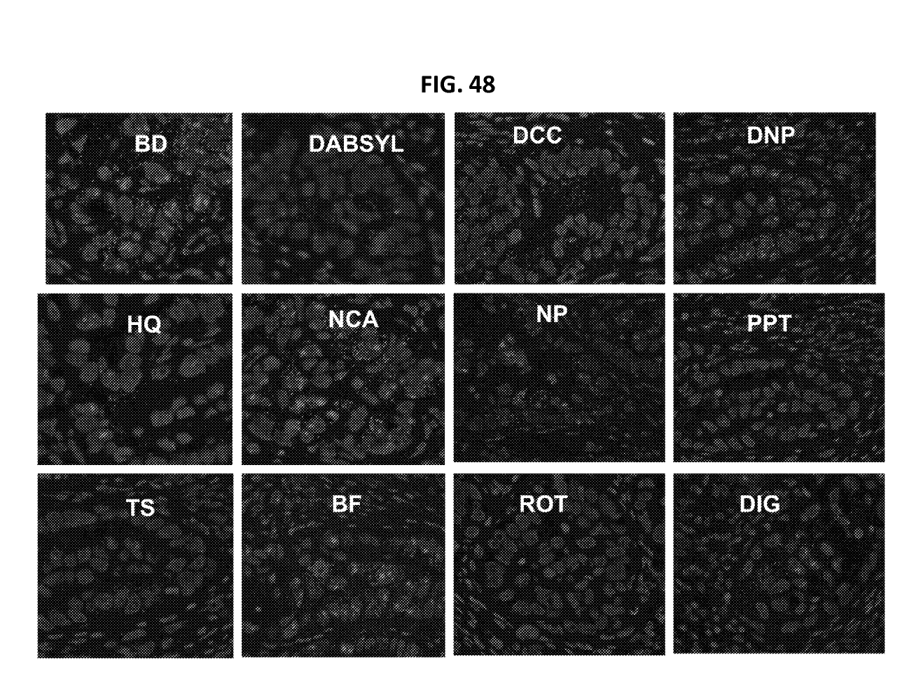

Formalin-fixed, paraffin-embedded Calu-3 human lung carcinoma xenograft tissue mounted on Superfrost™ slides was de-paraffinized and antigen retrieved using RiboClear denaturant, RiboCC reagent, and protease 3 (VMSI). Following retrieval, one drop (100 μL) of a DNP-labeled anti-sense or sense strand HER2 probe was dispensed onto a slide, denatured at 80° C. for 8 min, and hybridized at 65° C. for 6 hrs. Following hybridization slides were washed 3 times using 0.1×SSC at 75° C. for 8 min. DNP haptens were detected using native rabbit anti-DNP monoclonal antibody (VMSI #760-4139) dispensed onto the slide followed by TSA block (VMSI #760-4142) and HRP-conjugated goat anti-rabbit polyclonal antibodies (VMSI #760-4315). Tyramide signal amplification was performed as follows. One drop of a tyramide-hapten conjugate (10 μg/mL) was dispensed onto each slide followed by one drop TSA-H₂O₂ (VMSI #760-4141). The reactions were incubated 24 min; each tyramide conjugated hapten was detected using its cognate monoclonal antibody (5 μg/mL) followed by Qd655-conjugated goat anti-mouse polyclonal antibodies (VMSI #213-2194). As a result, performances of each tyramide-hapten conjugate/anti-hapten mAb system were evaluated individually and independent of probe or quantum dot conjugate variability. The procedure is illustrated schematically in FIG. 3B. Slides were counterstained using DAPI (VMSI #760-4196). The slides were dehydrated using gradient alcohols and coverslipped. The DAPI (VMSI #760-4196) and 655-nm signals were imaged using an Olympus fluorescent microscope fitted with a Spectral Imaging camera (Applied Spectral Imaging (ASI) Vista, Calif.) (FIG. 48).

Tissue Signal Quantification.

Fluorescent emission signals with QDot™ 655 were ranked by two blind readers. Results using a relative signal intensity scale (0 to 10) are included (FIG. 47).

FIG. 47 shows that there is significant variability in the fluorescent signal obtained from the haptens. Furthermore, as can be seen in FIG. 47, the performance of a particular hapten-tyramide conjugate could not be predicted from the performance of the corresponding haptenylated RNA probe. For example, the BF-tyramide conjugate produced a signal that was nearly twice as strong as the BF-labeled RNA probe. Conversely, the DNP-tyramide conjugate produced a signal that was significantly less than the DNP-labeled RNA probe. Surprisingly, the BD-, DIG-, HQ-, and NCA-tyramide conjugates all produced strong signals, while their respective haptenylated RNA probes produced little-to-no signal. Table 5 below provides a ranking of the haptens in each test.

TABLE 5

Ranking

| | Haptenylated | Hapten-Tyramide |
|---|---|---|
| Hapten | Probe | Conjugate |
| BD | 9 | 2 |
| BF | 3 | 1 |
| DABSYL | 4 | 6 |
| DCC | 1 | 4 |
| DIG | 8 | 3 |
| DNP | 2 | 4 |
| HQ | 12 | 5 |
| NCA | 11 | 4 |
| NP | 5 | 4 |
| PPT | 7 | 6 |
| ROT | 10 | 7 |
| TS | 6 | 6 |

### Example 4

### Hapten-Tyramide Conjugate Signals in an mRNA-ISH Assay

This example evaluates the signals obtained in an mRNA-ISH assay when tyramide signal amplification is performed using hapten-tyramide conjugates. Haptens were conjugated to tyramine via a polyethylene glycol linker to form a hapten-dPEG®₈-tyramide conjugate as described in Example 1.

Tissue Staining.

Formalin-fixed, paraffin-embedded Calu-3 xenograft tissue mounted on Superfrost slides was de-paraffinized and antigen retrieved using RiboClear denaturant, RiboCC reagent, and protease 3 (VMSI). Following retrieval, one drop (100 μl) of a hapten-labeled anti-sense or sense strand HER2 probe was dispensed onto a slide, denatured at 80° C. for 8 min, and hybridized at 65° C. for 6 hrs. Following hybridization slides were washed 3 times using 0.1×SSC at 75° C. for 8 min. The hapten-labeled probes were detected using the cognate anti-hapten monoclonal antibody conjugated to HRP at a concentration of 50 μg/mL. The HRP conjugate was dispensed onto the slide with TSA Block. Tyramide signal amplification was performed as follows: One drop of a hapten-tyramide conjugate (10 μg/mL was dispensed onto each slide followed by one drop TSA-H₂O₂ (Ventana). The reactions were incubated 24 min; each tyramide conjugated hapten was detected using its cognate monoclonal antibody conjugated to Qd655. The procedure is illustrated schematically in FIG. 3A. Slides were counterstained using DAPI. The slides were dehydrated using gradient alcohols and coverslipped. The DAPI and 655 nm signals were imaged using an Olympus fluorescent microscope fitted with a Spectral Imaging camera.

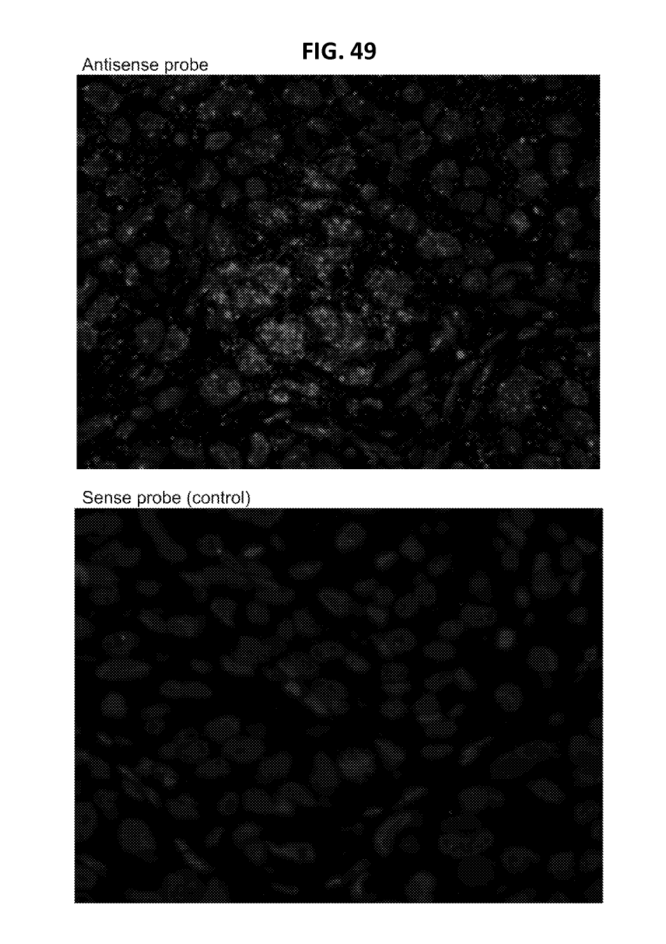

FIG. 49 illustrates the results obtained when the anti-sense and sense strand (control) HER2 probes were labeled with DNP, and detection was performed using MSxDNP-HRP (anti-hapten monoclonal antibody conjugated to HRP), DNP-dPEG®₈-tyramide conjugate, and MSxDNP-Qd655 (anti-DNP monoclonal antibody conjugated to Qd655).

### Example 5

### Multiplexed In Situ Hybridizations

This example evaluates the signals obtained in multiplexed mRNA-ISH assays of 18S rRNA and a breast cancer panel.

Probe Synthesis and Formulation:

ACTB, ER, HER2, Ki67, PR and 18S experimental anti-sense and control sense riboprobes chemically labeled with different haptens using Mirus linker arms (Label IT® linker) were prepared as directed by the manufacturer (Mirus Bio LLC, Madison, Wis.). Specifically, ER probes were labeled with benzofurazan (BF), HER2 probes were labeled with thiazolesulfonamide (TS), Ki67 probes were labeled with nitropyrazole (NP), and ACTB probes were labeled with 2,4-dinitrophenyl (DNP).

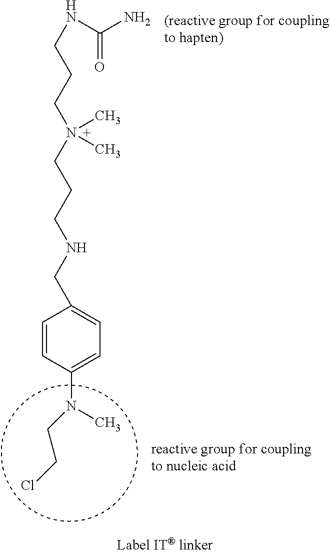

Labeling reactions were prepared according to the manufacturer's protocol (Lit. # ML012, rev. Mar. 31, 2005, accessed at the Mirus Bio website on Feb. 4, 2011) by combining the Amine Label IT® reagent (Kit # MIR 3900) and nucleic acid in a mass ratio of 0.2:1 to 0.8:1. For example, the Amine Label IT® reagent was reconstituted with 100 μL Reconstitution Solution to final concentration of 1 mg/mL linker. To label RNA probes, 37.5 μL deionized H₂O, 5 μL 10× Mirus Labeling Buffer A, 5 μL RNA probe solution (1 mg/mL), and 2.5 μL Amine Label IT® reagent were combined. The labeling reactions were incubated at 37° C. for 1 hour.

Labeled RNA was precipitated by adding 1.5 volumes of Ambion® lithium chloride precipitation solution (7.5 M lithium chloride, 50 mM EDTA, pH. 8.0, Applied Biosystems/Ambion, Austin, Tex., cat. # AM9480), and chilling the solution at −20° C. for 30 minutes. The solution was centrifuged in a microcentrifuge for 15 minutes, and the supernatant was discarded. The pellet was washed ice-cold 70% ethanol to remove residual salt. The labeled RNA was resuspended in nuclease-free water (Ambion).

The desired hapten was coupled to the free end of the Label IT® linker by reacting about 5 μg of labeled RNA probe with a 10 mM solution of the hapten-PEG₍₈₎ -NHS ester (prepared in anhydrous DMSO) and 100 mM NaHCO₃ (pH 8.5, freshly prepared) for one hour at room temperature in the dark. The hapten-labeled RNA probe was isolated by lithium chloride precipitation as previously described.

For the multiplexed breast panel in situ hybridization assay one hundred nanograms of each probe was suspended in 1 mL of Ribohybe™ (VMSI #760-104) solution and placed into a dispenser; for the model 18S multiplexed assay one nanogram of 18S probe labeled with various haptens was suspended in 1 mL of Ribohybe™ (VMSI #760-104) solution and placed into a dispenser.

Multiplexed In Situ Hybridizations (18S and Breast Panel):

Formalin-fixed, paraffin-embedded Calu-3, ZR75-1 and MCF-7 xenograft tissues mounted on Superfrost slides were de-paraffinized and antigen retrieved using RiboClear (VMSI #760-4125) denaturant, RiboCC VMSI #760-107) reagent, and protease 3 (VMSI #760-2020). Following retrieval, one drop (100 μL) of cocktailed anti-sense or sense strand probes labeled with distinct haptens was dispensed onto a slide, denatured at 80° C. for 8 min, and hybridized at 65° C. for 6 hrs. Following hybridization slides were washed three times using 0.1×SSC at 75° C. for 8 min; each hapten in the cocktail was detected sequentially as follows. Endogenous peroxidase activity was inactivated using PO inhibitor (VMSI #760-4143) and 10 ug/ml of HRP-conjugated anti-hapten monoclonal antibody dispensed onto the slide, incubated for 24 min. followed by TSA block (VMSI #760-4142). Tyramide signal amplification was accomplished by dispensing one drop of a tyramide-hapten conjugate (100 uM) on the slide followed by one drop TSA-H₂O₂ (VMSI #760-4141) and incubating the reaction for 24 min. The procedure was repeated to amplify each hapten in the probe cocktail. Amplified haptens were then detected using a cocktail of anti-hapten monoclonal antibodies each conjugated to a distinct Qdot. The sequential multiplexed procedure is illustrated schematically in FIGS. 5A-5B. Slides were counterstained using DAPI (VMSI #760-4196) and dehydrated using gradient alcohols and coverslipped. Probe cocktails comprised of control sense strand probes were used as negative controls for all experiments to determine background resulting from non-specific interactions.

Imaging:

The DAPI and Qdot signals were imaged using an Olympus fluorescent microscope fitted with a Spectral Imaging camera (Applied Spectral Imaging (ASI) Vista, Calif.). Images were captured using a 40× objective and ASI software package.

18S Multiplexed Assay:

18S RNA is expressed constitutively in all cells, making it a suitable model system and endogenous control for developing and testing multiplexed assays. Because 18S RNA is abundant in cells, very small amounts (e.g., picomoles) of several 18S RNA probes—each probe directed to the same target but labeled with different haptens—can be applied to a single tissue sample and will bind noncompetitively to the target 18S RNA sequence. Equimolar amounts of each probe are expected to result in substantially equal signals from each probe.

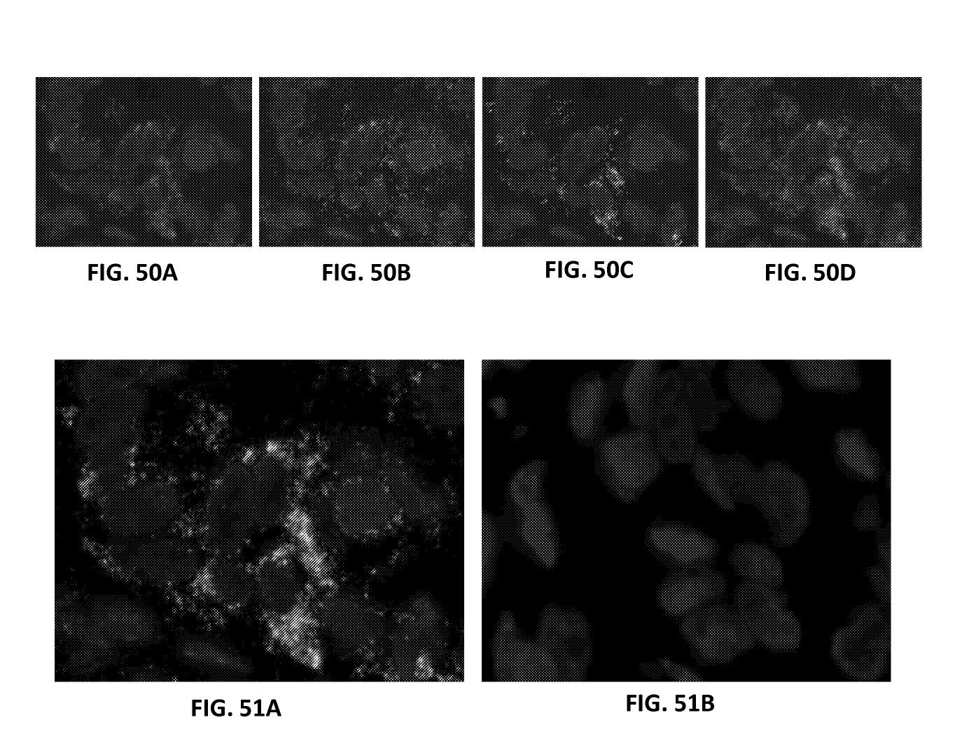

A multiplexed assay as described above was performed by hybridizing 18S probes labeled with DNP, BF, NP, and TS to Calu-30 xenograft cells. The DNP-, BF-, NP-, and TS-labeled probes were detected with quantum dots capable of emitting fluorescence at 655, 605, 585, and 565 nm, respectively. Specific reagents used in the model 18S multiplex reaction are detailed in Table 6. Each signal was detected individually at the appropriate wavelength, as shown in FIGS. 50A-D. The images were then combined into a single composite fluorescence image (FIG. 51A). As a negative control, similarly labeled sense-strand probes were utilized. A composite fluorescence image after the analogous four sense-strand probes were hybridized to the Calu-3 xenograft tissue shows no signal (FIG. 51B.)

TABLE 6

| PROBE | 18S | 18S | 18S | 18S |
|---|---|---|---|---|
| Hapten | BF | TS | NP | DNP |
| HRP | MSxBF | MSxDIG | MSxNP | MSxDNP |
| conjugate | | | | |
| Tyramide | TSA-BF | TSA-DIG | TSA-NP | TSA-DNP |
| conjugate | | | | |
| Anti-Hapten | MSxBF- | MSxDIG- | MSxNP- | MSxDNP- |
| Qdot | Qd605 | Qd565 | Qd585 | Qd655 |

Breast Panel Multiplexed Assay:

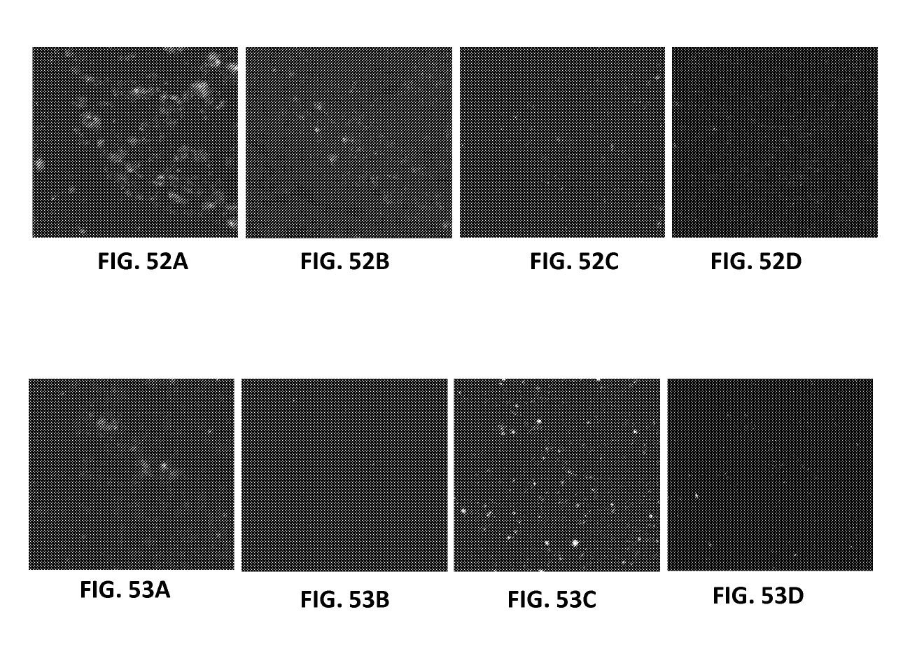

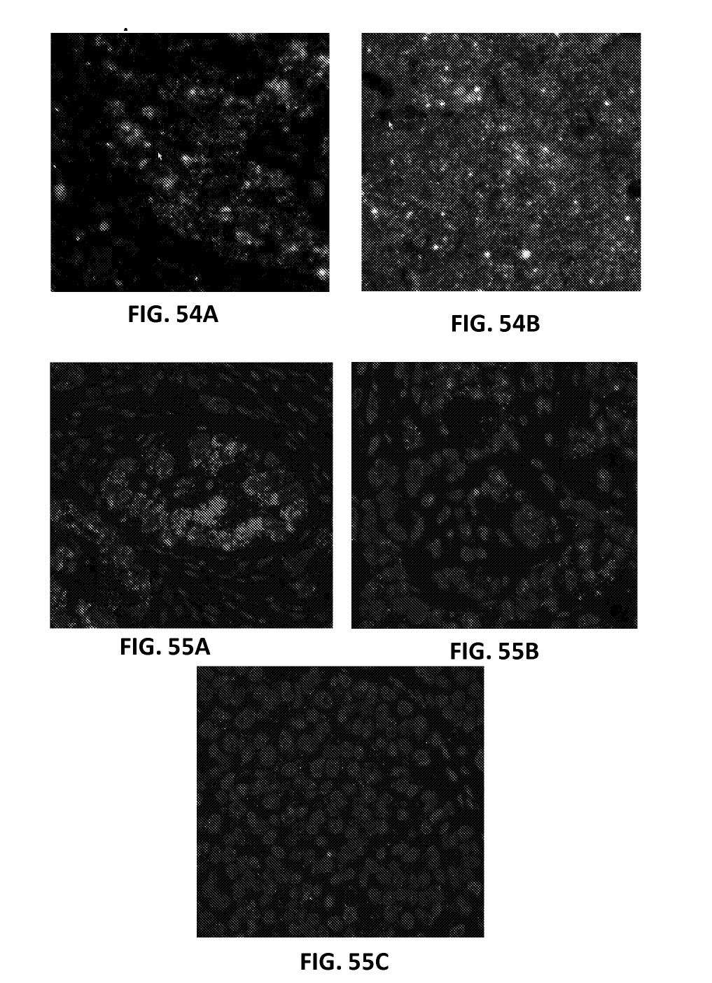

A multiplexed assay as described above was performed by hybridizing Calu-3 xenograft tissue and MCF-7 xenograft tissue samples with NP-labeled Ki67, TS-labeled HER2, BF-labeled ER, and DNP-labeled ACTB antisense RNA probes. The Ki67, HER2, ER, and ACTB probes were detected with quantum dots capable of emitting fluorescence at 525, 565, 605, and 655 nm, respectively. DAPI counterstaining of the nuclei was not performed. Specific reagents used in the breast panel multiplex hybridization are detailed in Table 7. Each QDot™ signal was detected individually at the appropriate wavelength, as shown in FIGS. 52A-D (Calu-3 xenograft tissue) and FIGS. 53A-D (MCF-7 xenograft tissue). Calu-3 xenograft cells are known to be HER2+, ER−, Ki67+/−, and ACTB⁺. As expected, strong signals were seen from the HER2 and ACTB probes, with a moderate signal from the Ki67 probe, and a very weak signal from the ER probe. MCF-7 xenograft cells are known to be HER2−, ER+, Ki67+/−, and ACTB+. As expected, strong signals were seen from the ER and ACTB probes, with a moderate signal from the Ki67 probe, and a very weak signal from the HER2 probe. Composite fluorescence images of the four probes are shown in FIGS. 54A (Calu-3 tissue) and 54B (MCF-7 tissue). Composite fluorescence images of the four negative control, analogous sense-strand RNA probes hybridized to Calu-3 and MCF-7 tissue showed no signal.

TABLE 7

| PROBE | ER | HER2 | Ki67 | ACTB |
|---|---|---|---|---|
| Hapten | BF | TS | NP | DNP |
| HRP | MSxBF | MSxDIG | MSxNP | MSxDNP |
| conjugate | | | | |
| Tyramide | TSA-BF | TSA-DIG | TSA-NP | TSA-DNP |
| conjugate | | | | |
| Anti-Hapten | MSxBF- | MSxDIG- | MSxNP- | MSxDNP- |
| Qdot | Qd605 | Qd565 | Qd525 | Qd655 |

Signal Quantification:

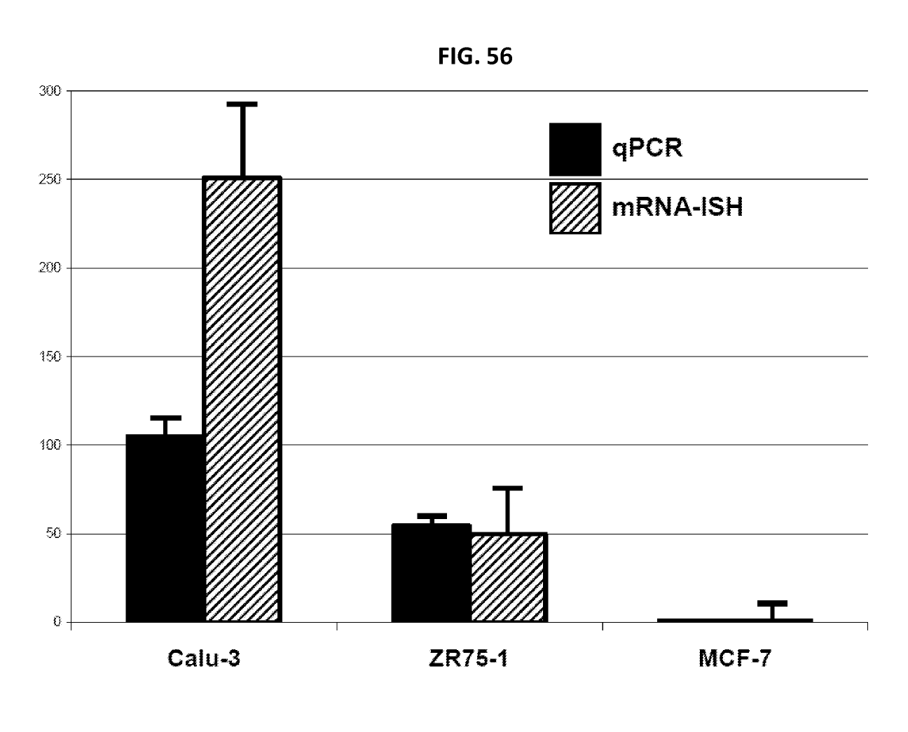

Calu-3, ZR75-1, and MCF-7 xenograft cells express high, low, and moderate amounts of HER2, respectively. However, ACTB expression is consistent in all cells, and can be used as an internal control. To determine the fluorescence signal's correlation with RNA expression, tissue samples were hybridized with DNP-labeled HER2 and TS-labeled ACTB antisense RNA probes as described above, and detected with monoclonal antibodies conjugated to Qd655 and Qd565, respectively. Spectral images were unmixed using the RawCubeViewer software package. Each QDot™ signal was thresholded to remove background and the number of pixels in the image above background counted using RawCubeViewer software. Ratios of HER2 to ACTB signals in each xenograft were determined by dividing the number of HER2 pixels by the number of ACTB pixels in each image. FIGS. 55A-C are fluorescence micrographs showing the fluorescence obtained from the HER2 probe hybridized with Calu-3, ZR75-1, and MCF-7 xenografts, respectively. As expected, the signal is much stronger in Calu-3 than ZR75-1 and MCF-7, and MCF-7 shows very little hybridization.

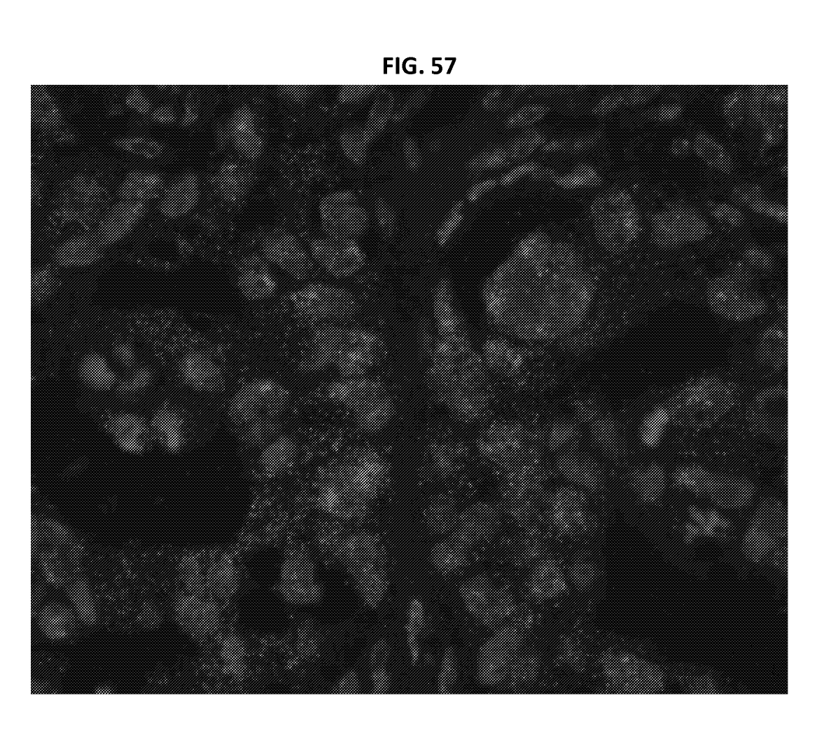

As a comparison, HER2 to ACTB ratios also were determined using quantitative RT-PCR (qPCR) as follows. Total mRNA was extracted from a ten micron section of each xenograft using a High Pure FFPE extraction kit (Roche). Each RNA sample was reverse transcribed using High Capacity RT kit (Applied Biosystems). Relative levels of HER2 and ACTB cDNA in each sample were determined using Taqman probes and Platinum DNA polymerase with UNG (Applied Biosystems). FIG. 56 is a graph depicting the HER2:ACTB mRNA ratios as detected by the qPCR and mRNA-ISH assays. As expected, the Calu-3 xenograft has a higher HER2:ACTB ratio than ZR75-1 and MCF-7 xenografts. The HER2:ACTB mRNA ratio of MCF-7 is near zero, as expected from MCF-7's known low HER2 expression. The HER2:ACTB mRNA ratio in Calu-3 tissue is approximately 2.5× greater as determined by mRNA-ISH compared to the ratio determined by qPCR. The differences can be explained by the gene expression pattern and the detection method. All cells express ACTB at a similar level. However, HER2 expression is stochastic, and only some cells in the tissue sample are expressing HER2 at any given time, as shown in FIG. 57. The mRNA-ISH assay detects only those cells that are expressing the genes of interest. A fluorescence image may focus on a region of interest in which HER2 expression is seen, producing a high HER2:ACTB ratio when the fluorescence signals are quantified. In contrast, when performing qPCR, all cells in the sample are destroyed, and the RNA is extracted and amplified via PCR. Thus, the qPCR tissue sample may include many cells that are not actively expressing HER2 at the time of the assay. The inclusion of inactive cells in the assay reduces the final amount of HER2RNA produced by the qPCR assay, thereby reducing the apparent HER2:ACTB ratio.

### Example 6

### Hapten-Tyramide Conjugate Signals in a Micro RNA-ISH Assay

This example evaluates the signals obtained in a a micro RNA (miRNA)-ISH assay when tyramide signal amplification is performed using hapten-tyramide conjugates. Haptens were conjugated to tyramine via a polyethylene glycol linker to form a hapten-dPEG®₈-tyramide conjugate as described in Example 1.

A. Evaluation of miR205LNA Probe on Lobular Breast Cancer Tissue Using a Tyramide-HQ Conjugate (Discovery Amp-HQ): The Following is the Adapted Procedure from the Ventana Discovery Ultra Instrument:

1. The paraffin coated tissue on the slide was heated to 65° C. for 4 minutes and treated with liquid cover slip (LCS). The slide was rinsed with EZPrep and had LCS reapplied. This process was done a total of three times at 65° C. in order to ensure deparaffinization of the tissue.

2. The slide was rinsed in reaction buffer and a 15 ug/mL solution of Proteinase K (Roche Applied Science #03115836001) diluted in a 5 mM Tris Buffer pH 7.3 with 1 mM EDTA was applied for 8 minutes at 37° C.

3. After 3 rinses with RiboWash (VMSI #760-105), 100 μL of the double DIG labeled miR205LNA probe (750 fmol, Exiqon #18099-15) was applied to the slide and heated to 80° C. for 8 minutes. After the 8 minute incubation, the slide hybridized at 60° C. for 1 hour.

4. After the hybridization of the probe, the slide underwent two stringency washes of 2×SSC at 60° C. for 4 minutes.

5. The slide was twice rinsed with reaction buffer and had 100 μL of a 2 μg/mL solution of Mouse anti-DIG (Roche Applied Science #11333062910) applied to the slide with liquid coverslip and incubated for 20 minutes at 37° C.

6. 100 uL of Amp Peroxidase Inhibitor (a component of VMSI #760-052) was applied to slide for 8 minutes.

7. The slide was then washed two times with reaction buffer, one drop of omniMap anti-Mouse HRP (VMSI #60-4310) incubated on the slide for 16 minutes at 37° C.

8. After washing the slide 3 times in reaction buffer, 100 μL of the Discovery Amp-HQ conjugate and one drop of Discovery Amplification H2O2 (both components of VMSI #760-052) was applied to the slide and incubated for 24 minutes at 37° C.

9. The slide was then washed 2 times with reaction buffer and 100 μL of the Discovery anti-HQ AP (VMSI #760-4521) was applied and incubated for 16 minutes on the slide at 37° C.

10. The slides were rinsed with EZ prep twice and had 100 μL of Activator CM, NBT CM and BCIP CM (all components of VMSI #760-161) added to the slide and incubated for 44 minutes.

11. The slide was rinsed three times in the reaction buffer before one drop of Red counterstain II (VMSI #780-2218) was applied the slide and incubated for 8 minutes.

12. Two more reaction buffer washes were applied to the slide to conclude the run.

13. The slide was removed from the instrument and treated to a detergent wash before manual application of a cover slip. The slide was viewed through a brightfield microscope.

B. Evaluation of miR205LNA Probe on Lobular Breast Cancer Tissue without Amplification:

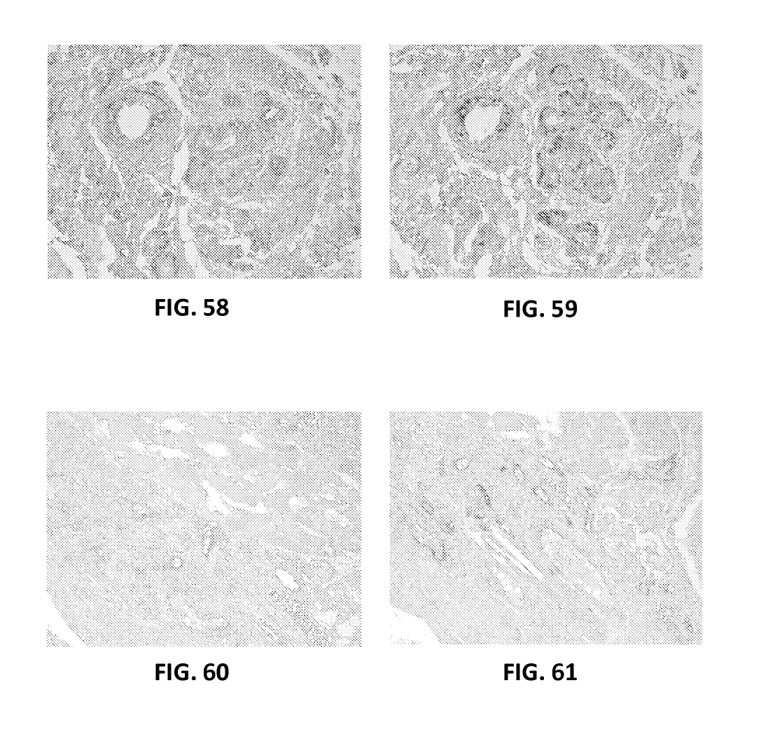

As a comparison, procedure in Part A above was repreated without tyramide-HQ amplification. Steps 1-5 were performed as described in Part A. Following step 5, the slide was washed 2 times with reaction buffer and 100 μL of the UltraMap anti-Mouse AP (VMSI #760-4312) was applied and incubated for 16 minutes on the slide at 37° C. Steps 10-13 of the procedure described in Part A then were performed. FIGS. 58-59 are photomicrographs illustrating the effect of hapten-tyramide conjugation on miR205 detection. FIG. 58 was obtained using the procedure in Part B (no amplification), and FIG. 59 was obtained using the procedure in Part A (amplification). The miR205 signal in FIG. 59 clearly is increased compared to the signal in FIG. 58.

C. Evaluation of miR126LNA Probe on Tonsil Tissue with Amplification Using a Tyramide-HQ Conjugate (Discovery Amp-HQ):

The procedure was the same as described above in Part A, with the following exceptions: 1) in step 3, a double DIG-labeled miR126LNA probe (Exiqon #88067-15) was used in place of the double DIG-labeled miR205LNA probe and hybridization was performed at 55° C.; 2) in step 4, the washes were performed at 55° C.

D. Evaluation of miR126LNA Probe on Tonsil Tissue without Amplification:

As a comparison, procedure in part C above was repreated without tyramide-HQ amplification. Steps 1-5 were performed as described in Part A, with the following exceptions: 1) in step 3, a double DIG-labeled miR126LNA probe (750 fmol, Exiqon #88067-15) was used in place of the double DIG-labeled miR205LNA probe and hybridization was performed at 55° C.; 2) in step 4, the washes were performed at 55° C. Following step 5, the slide was washed 2 times with reaction buffer and 100 μL of the UltraMap anti-Mouse AP (VMSI #760-4312) was applied and incubated for 16 minutes on the slide at 37° C. Steps 10-13 of the procedure described in Part A then were performed.

FIGS. 60-61 are photomicrographs illustrating the effect of hapten-tyramide conjugation on miR126 detection. FIG. 60 was obtained using the procedure in Part C (no amplification), and FIG. 61 was obtained using the procedure in Part D (amplification). The miR126 signal in FIG. 60 clearly is increased compared to the signal in FIG. 61.

The following U.S. patent, patent publications, and applications are assigned to Ventana Medical Systems, Inc., the assignee of the present application, and each is incorporated herein by reference: U.S. Pat. No. 7,695,929; U.S. Patent Publication No. 2007/0117153; U.S. Patent Publication No. 2006/0246524; U.S. Patent Publication No. 2006/0246423; U.S. patent application Ser. No. 12/154,472; U.S. Provisional Application No. 61/328,494; U.S. patent applications entitled Tyramine and Tyramine Derived Mass Tag Conjugate Compositions and Methods, filed on Jul. 2, 2010; and Enzymatic Amplified Mass Tags for Mass Spectrometric Tissue Imaging and Immunoassays, filed on Jul. 2, 2010.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A hapten conjugate, comprising:
a hapten selected from an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, 2,3,6,7-tetrahydro-11-oxo-1H,5H,11H-[1]benzopyrano[6,7,8-ij]quinolizine-10-carboxylic acid, or 7-diethylamino-3-carboxycoumarin;
a linker; and
a tyramine or a tyramine derivative.

2. (canceled)

3. The hapten conjugate according to claim 1 wherein the tyramine and/or tyramine derivative has the following general formula 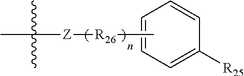
where R₂₅ is selected from hydroxyl, ether, amine, and substituted amine; R₂₆ is selected from alkyl, alkenyl, alkynyl, aryl, heteroaryl, —ORₘ, —NRₘ, and —SRₘ, where m is 1-20; n is 1-20; Z is selected from oxygen, sulfur, and NRₐ where Rₐ is selected from hydrogen, aliphatic, aryl, or alkyl aryl.

4. The hapten conjugate according to claim 3 wherein the tyramine and/or tyramine derivative has the following chemical structure

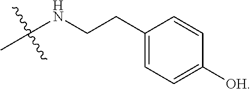 [SMILES: *NCCc1ccc(O)cc1]

5. The hapten conjugate according to claim 1 wherein the linker has the following general formula 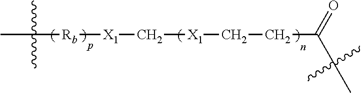
where each X₁ independently is selected from —CH₂, oxygen, sulfur, and —NR_{c} where R_{c} is selected from hydrogen, aliphatic, aryl, and aryl alkyl; R_{b} is selected from carbonyl and sulfoxyl; n is 1-20; and p is 0 or 1.

6. (canceled)

7. The hapten conjugate according to claim 1 wherein the linker has the following chemical structure

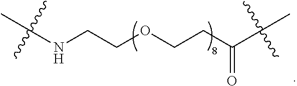

8.-11. (canceled)

12. The hapten conjugate according to claim 1 having a formula selected from the group consisting of

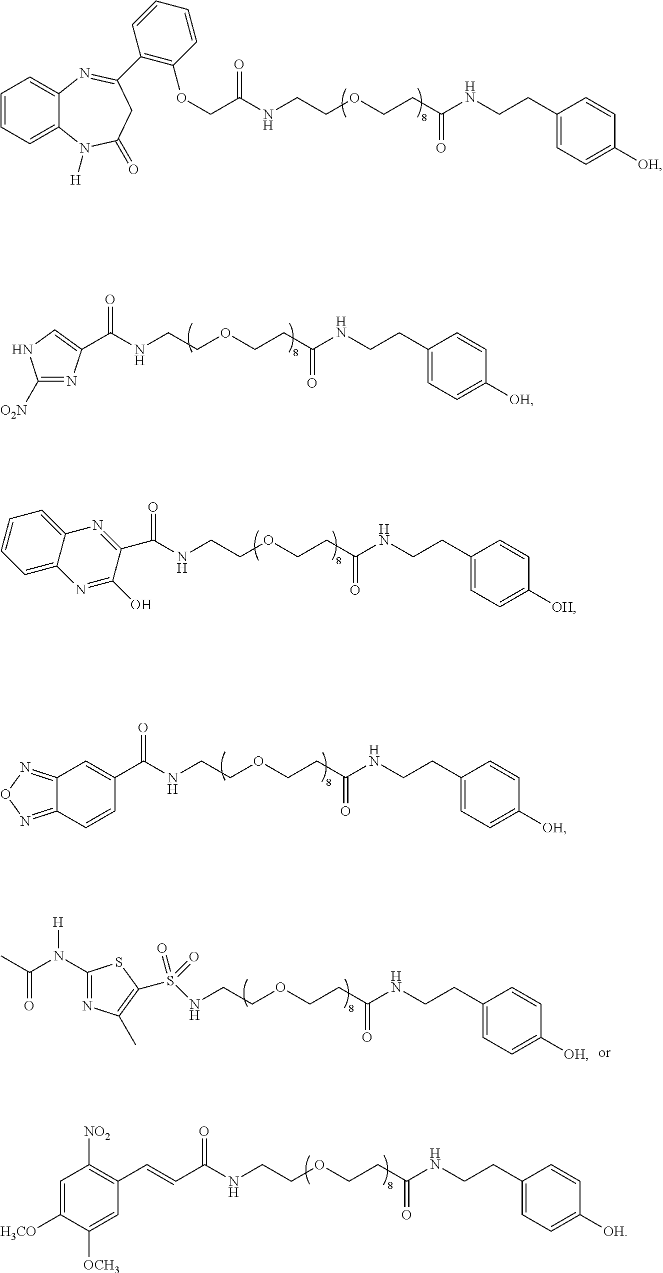

13.-32. (canceled)

33. A method, comprising:
(a) immobilizing a first peroxidase on a first target in a sample, wherein the first peroxidase is capable of reacting with a peroxidase-activatable aryl moiety;
(b) contacting the sample with a solution comprising a first hapten conjugate, the first hapten conjugate comprising a first hapten selected from an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, 2,3,6,7-tetrahydro-11-oxo-1H,5H,11H-[1]benzopyrano[6,7,8-ij]quinolizine-10-carboxylic acid, or 7-diethylamino-3-carboxycoumarin;
a linker; and
a tyramine or a tyramine derivative
(c) contacting the sample with a solution comprising peroxide, whereby the first hapten conjugate reacts with the first peroxidase and the peroxide, forming a covalent bond to the immobilized first peroxidase or proximal to the immobilized first peroxidase; and
(d) locating the first target in the sample by detecting the first hapten.

34. (canceled)

35. The method according to claim 33 wherein the peroxidase is conjugated to a moiety capable of recognizing and binding to the target.

36. The method according to claim 35 wherein the moiety is an antibody, nucleotide, oligonucleotide, protein, peptide or amino acid.

37.-41. (canceled)

42. The method according to claim 33, wherein detecting the first hapten of the first hapten conjugate further comprises:
contacting the sample with a first anti-hapten antibody capable of recognizing and binding to the first hapten of the first hapten conjugate and a first detectable label; and
detecting the first detectable label.

43. The method according to claim 42, wherein contacting the sample with a first anti-hapten antibody and a first detectable label comprises contacting the sample with a first anti-hapten antibody conjugate, wherein the first anti-hapten antibody conjugate comprises the first anti-hapten antibody and the first detectable label.

44. The method according to claim 42, wherein contacting the sample with a first anti-hapten antibody and a first detectable label comprises:
contacting the sample with the first anti-hapten antibody; and
contacting the sample with a first antibody conjugate, wherein the first antibody conjugate comprises an antibody capable of recognizing and binding to the first anti-hapten antibody and the first detectable label.

45. The method according to claim 42 wherein the first detectable label is an enzyme or a fluorescent label.

46.-49. (canceled)

50. The method according to claim 33 or claim 31 wherein the sample comprises two or more targets, the method further comprising:
after step (c), immobilizing a subsequent peroxidase on a subsequent target in the sample, wherein the subsequent peroxidase is capable of reacting with a peroxidase-activatable aryl moiety;
contacting the sample with a solution comprising a subsequent hapten conjugate, wherein the subsequent hapten conjugate comprises a subsequent hapten selected from an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, 2,3,6,7-tetrahydro-11-oxo-1H,5H,11H-[1]benzopyrano[6,7,8-ij]quinolizine-10-carboxylic acid, or 7-diethylamino-3-carboxycoumarin that is not the same as the first hapten or any other subsequent hapten, a linker, and a tyramine or a tyramine derivative;
contacting the sample with a solution comprising peroxide, whereby the subsequent hapten conjugate reacts with the subsequent peroxidase and the peroxide, forming a covalent bond to the immobilized subsequent peroxidase or proximal to the immobilized subsequent peroxidase; and
locating the two or more targets in the sample by detecting the first and subsequent haptens.

51. The method of claim 50, further comprising inactivating the first peroxidase before immobilizing the subsequent peroxidase.

52. The method of claim 33 wherein the sample comprises two or more targets, each target comprising a nucleic acid sequence, the method further comprising:
before step (a), immobilizing a first probe comprising DNA, RNA, or an oligonucleotide on the sample, wherein the first probe is labeled with a first hapten and is capable of recognizing and binding to the first target, and wherein the first hapten is selected from an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, or 7-diethylamino-3-carboxycoumarin;
before step (a), immobilizing a subsequent probe comprising DNA, RNA, or an oligonucleotide on the sample, wherein the subsequent probe is labeled with a subsequent hapten and is capable of recognizing and binding to a subsequent target, and wherein the subsequent hapten is not the same as the first hapten or any other subsequent hapten, and wherein the subsequent hapten is selected from an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, or 7-diethylamino-3-carboxycoumarin;
wherein immobilizing the first peroxidase in step (a) comprises contacting the sample with a first anti-hapten antibody-peroxidase conjugate comprising a first anti-hapten antibody and a first peroxidase, wherein the first anti-hapten antibody is capable of recognizing and binding to the first hapten, and wherein the first peroxidase is capable of reacting with a peroxidase-activatable aryl moiety;
after step (c), contacting the sample with a subsequent anti-hapten antibody-peroxidase conjugate comprising a subsequent anti-hapten antibody and a subsequent peroxidase, wherein the subsequent anti-hapten antibody is capable of recognizing and binding to the subsequent hapten, and wherein the subsequent peroxidase is capable of reacting with a peroxidase-activatable aryl moiety;
contacting the sample with a solution comprising a subsequent hapten conjugate according to any one of claims 1-29, wherein the subsequent haptenconjugate comprises a subsequent hapten that is not the same as the first hapten or any other subsequent hapten;
contacting the sample with a solution comprising peroxide, whereby the subsequent hapten conjugate reacts with the subsequent peroxidase and the peroxide, forming a covalent bond to the immobilized subsequent peroxidase or proximal to the immobilized subsequent peroxidase; and
locating the two or more targets in the sample by detecting the first and subsequent haptens.

53. The method of claim 52, where locating the two or more targets in the sample further comprises:
contacting the sample with a solution comprising a first anti-hapten antibody-quantum dot conjugate and a subsequent anti-hapten antibody-quantum dot conjugate, wherein the first anti-hapten antibody-quantum dot conjugate comprises a first antibody capable of recognizing and binding to the first hapten of the first hapten-tyramide conjugate and a first quantum dot, and the subsequent anti-hapten antibody-quantum dot conjugate comprises a subsequent antibody capable of recognizing a binding to the subsequent hapten of the subsequent hapten-tyramide conjugate and a subsequent quantum dot, wherein the subsequent quantum dot is not the same as the first quantum dot or any other subsequent quantum dot; and
detecting fluorescence from the first and subsequent quantum dots.

54. The method of claim 52, further comprising inactivating the first anti-hapten antibody-peroxidase conjugate before contacting the sample with the subsequent anti-hapten antibody-peroxidase conjugate.

55. The method of claim 52, where the sample is obtained from a subject suspected of having breast cancer, and at least one of the first probe or the subsequent probe is an anti-sense RNA probe capable of hybridizing to HER2 mRNA, ER mRNA, Ki-67 mRNA, or PGR mRNA.
